(19) United States
(12) Patent Application Publication
(10) Pub. No.: US 2024/0294543 A1
(43) Pub. Date: Sep. 5, 2024

(54) CYCLOHEPTA-THIENO-DIAZEPINE DERIVATIVES AS GABA A GAMMA1 PAM

(71) Applicant: Hoffmann-La Roche Inc., Little Falls, NJ (US)

(72) Inventors: Bjoern BARTELS, Schopfheim (DE); Giuseppe CECERE, Basel (CH); Guido GALLEY, Rheinfelden (DE); Luca GOBBI, Buus (CH); Martin GOEBEL, Weinheim (DE); Maria-Clemencia HERNANDEZ, Delémont (CH); Andrés Miguel OLIVARES MORALES, Binningen (CH); Hasane RATNI, Habsheim (FR); Michael REUTLINGER, Freiburg (DE); Valerie RUNTZ-SCHMITT, Rixheim (FR)

(73) Assignee: Hoffmann-La Roche Inc., Little Falls, NJ (US)

(21) Appl. No.: 18/626,665

(22) Filed: Apr. 4, 2024

Related U.S. Application Data
(63) Continuation of application No. PCT/EP2022/077524, filed on Oct. 4, 2022.

(30) Foreign Application Priority Data
Oct. 6, 2021 (EP) 21201281.9

(51) Int. Cl. C07D 495/14; A61K 31/551; C07D 495/22

(52) U.S. Cl. CPC C07D 495/14; A61K 31/551; C07D 495/22

(57) ABSTRACT

The invention provides novel heterocyclic compounds having the general formula (I), and pharmaceutically acceptable salts thereof, wherein the variables are as described herein.

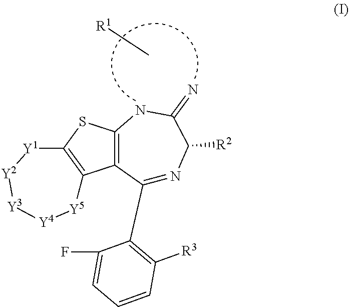

Further provided are pharmaceutical compositions including the compounds, processes of manufacturing the compounds and methods of using the compounds as medicaments, in particular methods of using the compounds for the treatment or prevention of acute neurological disorders, chronic neurological disorders and/or cognitive disorders.

### CROSS REFERENCE TO RELATED APPLICATIONS

This application is a continuation of International Patent Application No. PCT/EP2022/077524, filed Oct. 4, 2022, which claims priority to European Patent Application No. 21201281.9 filed Oct. 6, 2021, both of which are incorporated herein by reference in their entirety.

### FIELD OF THE INVENTION

The invention relates to organic compounds useful for therapy or prophylaxis in a mammal, and in particular to new cyclohepta-thieno-diazepine derivatives that exhibit activity as GABAA γ1 receptor positive allosteric modulators (PAMs) and are thus useful for the treatment or prophylaxis of GABAA γ1 receptor related diseases or conditions.

### BACKGROUND OF THE INVENTION

Receptors for the major inhibitory neurotransmitter, gamma-aminobutyric acid (GABA), are divided into two main classes: (1) GABA_{A} receptors, which are members of the ligand-gated ion channel superfamily and (2) GABA_{B} receptors, which are members of the G-protein linked receptor family. The GABA_{A} receptor complex which is a membrane-bound heteropentameric protein polymer is composed principally of α, β and γ subunits. GABA_{A} receptors are ligand-gated chloride channels and the principal mediators of inhibitory neurotransmission in the human brain.

There are 19 genes encoding for GABA_{A} receptor subunits that assemble as pentamers with the most common stoichiometry being two α, two β and one γ subunit. GABA_{A} subunit combinations give rise to functional, circuit, and behavioral specificity. GABA_{A} receptors containing the γ1 subunit (GABA_{A} γ1) are of particular interest due to their enriched expression in the limbic system and unique physiological and pharmacological properties. The GABA_{A} γ1 subunit-containing receptors, while less abundant (around 5-10% of total expression of GABA_{A} receptors in the brain) than γ2 subunit-containing receptors exhibit an enriched brain mRNA and protein distribution in key brain areas such as extended amygdala (central, medial, and bed nucleus of the stria terminalis), lateral septum, hypothalamus, and pallidum/nigra. These structures form the interconnected core of a subcortical limbic circuit regulating motivated social and affective behaviors. In abnormal or disease conditions, hyper-recruitment of this circuit promotes anxiety, arousal, aggression, fear and defense while inhibiting foraging and social interactions.

Hyperactivity in limbic cortical regions (known to form a coordinated functional network with extended amygdala/hypothalamus regions) which are key areas for processing of social and emotionally relevant stimuli, is the common hallmark of a variety of psychiatric, neurological, neurodevelopmental, neurodegenerative, mood, motivational and metabolic disorders. In such a disease state, and given the characteristic anatomical distribution of the γ1 subunit-containing GABA_{A} receptors, a GABA_{A} γ1 positive allosteric modulator (PAM) may be an effective treatment as a symptomatic or disease-modifying agent.

Multiple lines of evidence suggest that an imbalance between excitatory/inhibitory (E/I) neurotransmission arising from dysfunction of GABAergic signaling system, the main inhibitory neurotransmitter system in the brain, to be at the core of the pathogenesis a variety of CNS disorders. Given the distribution and function of GABA_{A} γ1 subunit-containing receptors in the CNS, they are very attractive targets for restoring levels of inhibition within key brain circuits and consequently the E/I balance in these conditions.

A CNS disorders of particular interest in the context of the present invention is autism spectrum disorder (ASD), including its core symptoms and associated comorbidities, such as anxiety and irritability, social anxiety disorder (social phobia) and generalized anxiety disorder. ASD is a complex, heterogeneous neurodevelopmental disorder characterized by impairments in two core domains: impairments in social interaction and communication, and presence of repetitive or restricted behaviors, interests, or activities (American Psychiatric Association 2013).

No approved pharmacological treatment exists for core symptoms of social deficits and restricted/repetitive behaviour of ASD, while only inadequate therapeutic options are available for most of ASD's affective and physiological co-morbidities. As a result, this disorder continues to be an area of high unmet medical need. Current approved treatments for associated symptoms of ASD are limited to the antipsychotics (Risperidone and Aripiprazole) indicated for the treatment of irritability associated with ASD symptoms. Emerging evidence suggests that the GABAergic system, the main inhibitory neurotransmitter system in the brain, plays a key role in the pathophysiology of ASD.

Both genetic and imaging studies using positron emission tomography study (PET) and magnetic resonance spectroscopy (MRS) suggest alterations in GABAergic signaling in ASD. The gene encoding GABA_{A} γ1, GABRG1, is located on chromosome 4 (mouse Chr.5) in a cluster with genes encoding α2, α4 and β1 GABA_{A} receptor subunits. Rare CNVs, including inversion of chromosome 4p12 disrupting GABRG1 have been observed in autistic siblings (Horike et al., 2006), as well as GABRG1 loss in one case of ADHD. Mutations in 4p12 gene cluster have been linked to increased risk of anxiety, substance abuse and eating disorders—providing a link between GABRG1/4p12 and affective dysfunction. MRS studies found altered GABA levels in ASD and in particular some recent studies showed reduced GABA and altered somatosensory function in children with ASD and. In line with these observations, a reduced number of inhibitory interneurons were found from postmortem tissues of ASD and TS patients. Furthermore, reduced GABA synthesizing enzymes, glutamic acid decarboxylase (GAD) 65 and 67 were found in parietal and cerebellar cortices of patients with autism. Strong evidence in humans points to specific dysfunction in ASD of the limbic cortical regions known to form a coordinated functional network with GABA_{A} γ1 subunit-containing extended amygdala/hypothalamus regions. These areas: Cortical/lateral amygdala, Insula, PFC, and Cingulate are recognized key for processing of social and emotionally relevant stimuli. While subcortical subnuclei that form specific partnerships with these areas, coordinating behavioural outcomes, are often difficult to study due to spatial resolution limitations, many lines of evidence point to hyper-recruitment of these cortical- to sub cortical connections in ASD. Moreover, recent high resolution studies provide a clear link between extended amygdala activity/functional connectivity and emotional state. Targeting such highly specified limbic subcortical regions, which exhibit substantial molecular and cellular diversity compared to the neocortex, will create a precision entry point for safe and specific therapeutic modulation of ASD-affected socio-affective circuits, while avoiding broad modulation of global brain state. Enhancement of GABA_{A} receptor activity by non-selective BZDs have been shown to ameliorate behavioral deficits in mouse models of ASD, however very narrow therapeutic margins were observed due to sedation mediated by the GABA_{A} α1γ2 subtype. These findings support the notion that rebalancing of GABAergic transmission via GABA_{A} γ1 receptors can improve symptoms in ASD without the side effects of non-selective benzodiazepines.

### SUMMARY OF THE INVENTION

Compounds of the present invention are selective GABA_{A} γ1 receptor positive allosteric modulators (PAMs) that selectively enhance the function of γ1-containing GABA_{A} receptors by increasing GABAergic currents (influx of chloride) at a given concentration (e.g. EC₂₀) of gamma amino butyric acid (GABA). The compounds of the present invention have high PAM efficacy and binding selectivity for the γ1-containing subtypes (α5γ1, α2γ1, α1γ1) relative to the γ2-containing subtypes (e.g. α1γ2, α2γ2, α3γ2 and α5γ2). As such, compounds of the present invention are strongly differentiated from classical benzodiazepine drugs such as Alprazolam, Triazolam, Estazolam, and Midazolam, which are selective for the γ2-containing GABA_{A} subtypes and possess low affinity for the γ1-containing subtypes. Compatible with the γ1-subtypes brain distribution, selective GABA_{A} γ1 PAMs will restore GABAergic signaling in key brain regions (e.g. extended amygdala: central, medial, and bed nucleus of the stria terminalis, lateral septum, hypothalamus, and pallidum/nigra) without the side-effects of non-selective GABA_{A} modulators (e.g. benzodiazepines).

In view of the above, the selective GABA_{A} γ1 PAMs described herein and their pharmaceutically acceptable salts and esters are useful, alone or in combination with other drugs, as disease-modifying or as symptomatic agents for the treatment or prevention of acute neurological disorders, chronic neurological disorders and/or cognitive disorders, including autism spectrum disorders (ASD), Angelman syndrome, age-related cognitive decline, Rett syndrome, Prader-Willi syndrome, amyotrophic lateral sclerosis (ALS), fragile-X disorder, negative and/or cognitive symptoms associated with schizophrenia, tardive dyskinesia, anxiety, social anxiety disorder (social phobia), panic disorder, agoraphobia, generalized anxiety disorder, disruptive, impulse-control and conduct disorders, Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurodegenerative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar

In a first aspect, the present invention provides a compound of formula (I)

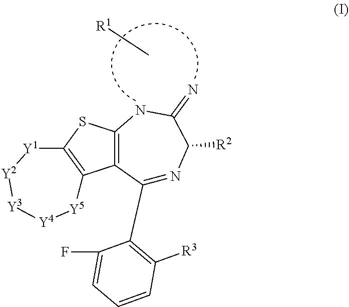

or a pharmaceutically acceptable salt thereof, wherein the variables are as defined herein.

In one aspect, the present invention provides a process of manufacturing the compounds of formula (I) described herein, wherein said process is as described in any one of Schemes 1 to 15 herein.

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in a method for treating or preventing acute neurological disorders, chronic neurological disorders and/or cognitive disorders in a subject.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl include methyl and ethyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. Particularly preferred, yet non-limiting examples of alkoxy are ethoxy and tert-butoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, and spiro[2.3]hexan-5-yl.

The term “heterocyclyl” or “heterocycloalkyl” refers to a saturated or partly unsaturated mono- or bicyclic, preferably monocyclic ring system of 3 to 14 ring atoms, preferably 3 to 10 ring atoms, more preferably 3 to 8 ring atoms, most preferably 3 to 6 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl; azetidin-2-yl; oxetan-3-yl; oxetan-2-yl; piperidyl; piperazinyl; pyrrolidinyl; 2-oxopyrrolidin-1-yl; 2-oxopyrrolidin-3-yl; 5-oxopyrrolidin-2-yl; 5-oxopyrrolidin-3-yl; 2-oxo-1-piperidyl; 2-oxo-3-piperidyl; 2-oxo-4-piperidyl; 6-oxo-2-piperidyl; 6-oxo-3-piperidyl; 1-piperidinyl; 2-piperidinyl; 3-piperidinyl; 4-piperidinyl; morpholino (e.g., morpholin-2-yl or morpholin-3-yl); thiomorpholino, pyrrolidinyl (e.g., pyrrolidin-3-yl); 3-azabicyclo[3.1.0]hexan-6-yl; 2,5-diazabicyclo[2.2.1]heptan-2-yl; 2-azaspiro[3.3]heptan-2-yl; 2,6-diazaspiro[3.3]heptan-2-yl; and 2,3,3a,4,6,6a-hexahydro-1H-pyrrolo[3,4-c]pyrrol-5-yl. A preferred, yet non-limiting example of heterocyclyl is azetidine.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, pyridazinyl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, pyrazin-3-yl, pyrazin-4-yl, imidazol-1-yl, 1H-imidazol-2-yl, 1H-imidazol-4-yl, 1H-imidazol-5-yl, oxazol-2-yl, oxazol-4-yl, oxazol-5-yl, thiazol-4-yl, 1,2,4-oxadiazol-3-yl, 1H-triazol-5-yl, 1H-triazol-4-yl, and triazol-1-yl. Most preferably, “heteroaryl” refers to pyridazinyl.

The term “hydroxy” refers to an —OH group.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Non-limiting examples of haloalkyl are fluoromethyl, difluoromethyl, trifluoromethyl, trifluoroethyl, 2-fluoroethyl, and 2,2-difluoroethyl. Particularly preferred, yet non-limiting examples of haloalkyl are difluoromethyl, trifluoromethyl, and 2-fluoroethyl.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl, hydroxyethyl (e.g. 2-hydroxyethyl), 2-hydroxy-2-methyl-propyl, and 3-hydroxy-3-methyl-butyl.

The term “hydroxycycloalkyl” refers to an cycloalkyl group, wherein at least one of the hydrogen atoms of the cycloalkyl group has been replaced by a hydroxy group. Preferably, “hydroxycycloalkyl” refers to a cycloalkyl group wherein 1, 2 or 3 hydrogen atoms, most preferably 1 hydrogen atom of the cycloalkyl group have been replaced by a hydroxy group. Preferred, yet non-limiting examples of hydroxyalkyl are hydroxcyclobutyl (e.g., 3-hydroxycyclobutyl) and hydroxycyclopropyl (e.g., 1-hydroxycyclopropyl).

The term “halohydroxyalkyl” refers to an alkyl group, wherein at least one of the hydrogen atoms of the alkyl group has been replaced by a hydroxy group and at least one of the hydrogen atoms of the alkyl group has been replaced by a halogen atom. Preferably, “halohydroxyalkyl” refers to an alkyl group wherein 1, 2 or 3 hydrogen atoms, most preferably 1 hydrogen atom of the alkyl group have been replaced by a hydroxy group and 1, 2 or 3 hydrogen atoms, most preferably 3 hydrogen atoms of the alkyl group have been replaced by a halogen atom. A preferred, yet non-limiting example of halohydroxyalkyl is 3,3,3-trifluoro-2-hydroxy-2-methyl-propyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as formic acid, acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, formates, lactates (in particular derived from L-(+)-lactic acid), tartrates (in particular derived from L-(+)-tartaric acid) and trifluoroacetates.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” or “prevention” as used herein includes: preventing or delaying the appearance of clinical symptoms of the state, disorder or condition developing in a subject and especially a human that may be afflicted with or predisposed to the state, disorder or condition but does not yet experience or display clinical or subclinical symptoms of the state, disorder or condition.

The term “subject” as used herein includes both humans and non-humans and includes but is not limited to humans, non-human primates, canines, felines, murines, bovines, equines, and porcines.

In a particularly preferred embodiment, the term “subject” refers to humans.

The abbreviation uM means microMolar and is equivalent to the symbol μM.

The abbreviation uL means microliter and is equivalent to the symbol μL.

The abbreviation ug means microgram and is equivalent to the symbol μg.

### Compounds of the Invention

In a first aspect, the present invention provides a compound of formula (I)

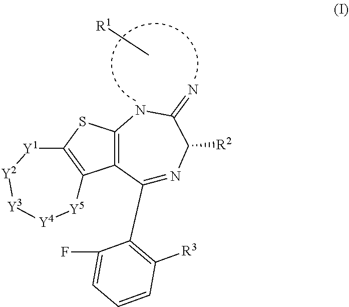

or a pharmaceutically acceptable salt thereof, wherein:

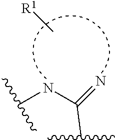

is selected from:

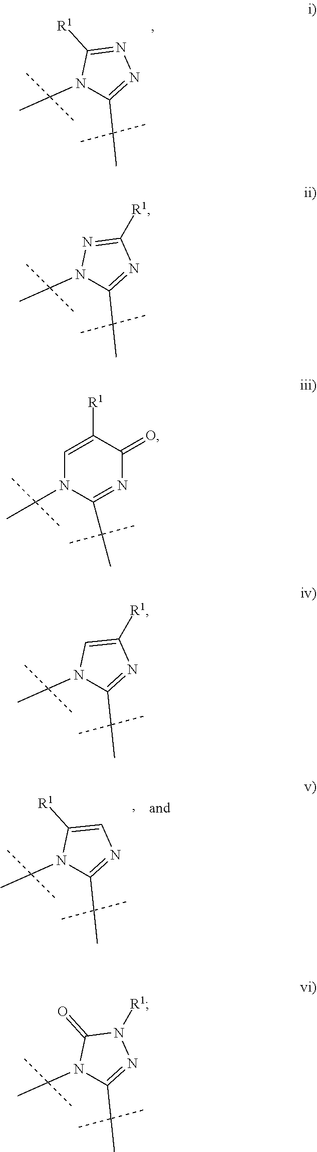

R¹ is selected from hydrogen, C₁-C₆-alkyl, halo-C₁-C₆-alkyl, R^{1a}R^{1b}NC(O)—, C₃-C₁₀-cycloalkyl, 3-14-membered heteroaryl;
R^{1a} is selected from C₁-C₆-alkyl, hydroxy-C₁-C₆-alkyl, halo-C₁-C₆-alkyl, halo-hydroxy-C₁-C₆-alkyl, C₃-C₁₀-cycloalkyl, hydroxy-C₃-C₁₀-cycloalkyl-C₁-C₆-alkyl-; and R^{1b} is hydrogen; or
R^{1a} and R^{1b}, taken together with the nitrogen atom to which they are attached, form a 3-14-membered heterocycle which is optionally substituted with one substituent selected from halogen, hydroxy, and C₁-C₆-alkoxy;
R² is selected from hydrogen and C₁-C₆-alkyl;
R³ is selected from chloro and fluoro;
Y¹ to Y⁵ are each independently selected from O, S, and CR⁴R⁵, provided that at most two of Y¹ to Y⁵ are O or S and that an Y being O or S is not bound to another Y also being O or S; and
R⁴ and R⁵ are each independently selected from hydrogen, deuterium, halogen, and hydroxy.

In one embodiment, the present invention provides a compound of formula (I) as described herein,

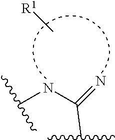

or a pharmaceutically acceptable salt thereof, wherein is selected from:

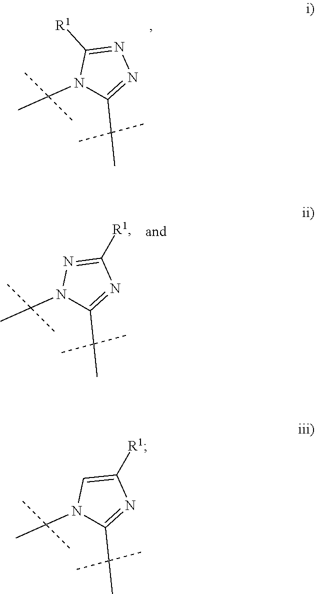

R¹ is selected from C₁-C₆-alkyl and R^{1a}R^{1b}NC(O)—;
R^{1a} is selected from hydroxy-C₁-C₆-alkyl and halo-hydroxy-C₁-C₆-alkyl; and
R^{1b} is hydrogen.

In a preferred, the present invention provides a compound of formula (I) as described herein, or a pharmaceutically acceptable salt thereof, wherein

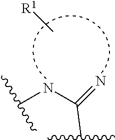

is selected from:

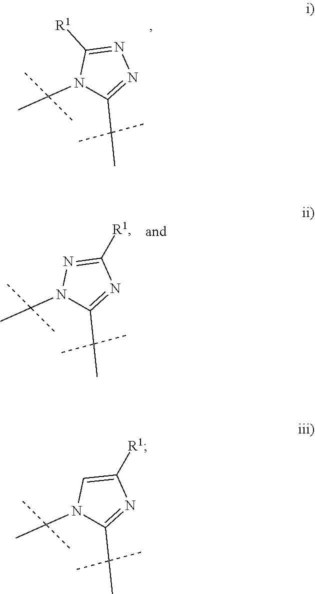

R¹ is selected from methyl and R^{1a}R^{1b}NC(O)—;
R^{1a} is selected from 2-hydroxy-2-methyl-propyl and 3,3,3-trifluoro-2-hydroxy-2-methyl-propyl; and
R^{1b} is hydrogen.

In one embodiment, the present invention provides a compound of formula (I) as described herein, or a pharmaceutically acceptable salt thereof, wherein

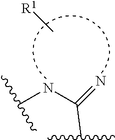

is selected from:

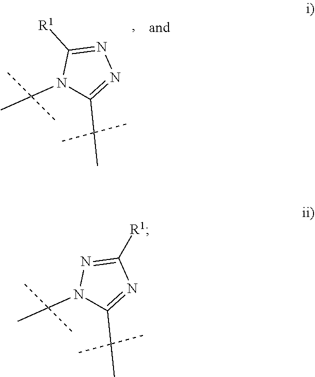

wherein R¹ is as described herein.

In one embodiment, the present invention provides a compound of formula (I) as described herein, or a pharmaceutically acceptable salt thereof, wherein

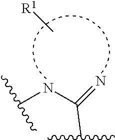

is

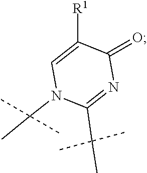

wherein R¹ is as described herein.

In one embodiment, the present invention provides a compound of formula (I) as described herein, or a pharmaceutically acceptable salt thereof, wherein

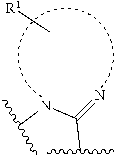

is

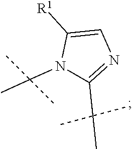

wherein R¹ is as described herein.

In one embodiment, the present invention provides a compound of formula (I) as described herein, or a pharmaceutically acceptable salt thereof, wherein

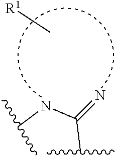

is

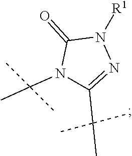

wherein R¹ is as described herein.

In a preferred embodiment, the present invention provides a compound of formula (I) as described herein, or a pharmaceutically acceptable salt thereof, wherein

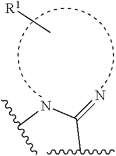

is

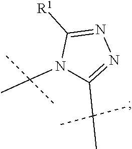

wherein R¹ is as described herein.

In a preferred embodiment, the present invention provides a compound of formula (I) as described herein, or a pharmaceutically acceptable salt thereof, wherein

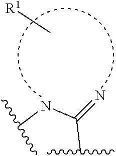

is

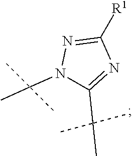

wherein R¹ is as described herein.

In a particularly preferred embodiment, the present invention provides a compound of formula (I) as described herein, or a pharmaceutically acceptable salt thereof, wherein

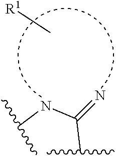

is

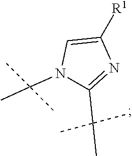

wherein R¹ is as described herein.

In a preferred embodiment, the present invention provides a compound of formula (I) as described herein, or a pharmaceutically acceptable salt thereof, wherein:
R¹ is selected from C₁-C₆-alkyl and R^{1a}R^{1b}NC(O)—;
R^{1a} is selected from hydroxy-C₁-C₆-alkyl and halo-hydroxy-C₁-C₆-alkyl; and
R^{1b} is hydrogen.

In a particularly preferred, the present invention provides a compound of formula (I) as described herein, or a pharmaceutically acceptable salt thereof, wherein
R¹ is selected from methyl and R^{1a}R^{1b}NC(O)—;
R^{1a} is selected from 2-hydroxy-2-methyl-propyl and 3,3,3-trifluoro-2-hydroxy-2-methyl-propyl; and
R^{1b} is hydrogen.

In a further particularly preferred, the present invention provides a compound of formula (I) as described herein, or a pharmaceutically acceptable salt thereof, wherein
R¹ is R^{1a}R^{1b}NC(O)—;
R^{1a} is selected from 2-hydroxy-2-methyl-propyl; and
R^{1b} is hydrogen.

In one embodiment, the present invention provides a compound of formula (I) as described herein, or a pharmaceutically acceptable salt thereof, wherein R² is C₁-C₆-alkyl.

In a preferred embodiment, the present invention provides a compound of formula (I) as described herein, or a pharmaceutically acceptable salt thereof, wherein R² is methyl.

In a preferred embodiment, the present invention provides a compound of formula (I) as described herein, or a pharmaceutically acceptable salt thereof, wherein R³ is fluoro.

In one embodiment, the present invention provides a compound of formula (I) as described herein, or a pharmaceutically acceptable salt thereof, wherein:
Y¹, Y³, Y⁴, and Y⁵ are each independently selected from O and CR⁴R⁵, provided that at most two of Y¹, Y³, Y⁴, and Y⁵ are O and that an Y being O is not bound to another Y also being O;
Y² is CR⁴R⁵;
R⁴ is selected from hydrogen, deuterium, halogen, and hydroxy; and
R⁵ is selected from hydrogen, deuterium, and halogen.

In a preferred embodiment, the present invention provides a compound of formula (I) as described herein, or a pharmaceutically acceptable salt thereof, wherein:
Y¹ and Y⁴ are each independently selected from O and CR⁴R⁵;
Y², Y³, and Y⁵ are each CR⁴R⁵;
R⁴ is selected from hydrogen and halogen; and
R⁵ is hydrogen.

In a particularly preferred embodiment, the present invention provides a compound of formula (I) as described herein, or a pharmaceutically acceptable salt thereof, wherein:
Y¹ and Y⁴ are each independently selected from O and CR⁴R⁵;
Y², Y³, and Y⁵ are each CR⁴R⁵;
R⁴ is selected from hydrogen and fluoro; and
R⁵ is hydrogen.

In one embodiment, the present invention provides a compound of formula (I) as described herein, or a pharmaceutically acceptable salt thereof, wherein:
Y¹ is selected from CH₂, CH—F, CH—OH, and O;
Y² is selected from CH₂, CD₂, CH-Me, CH—F, CF₂, and CH—OH;
Y³ is selected from CH₂, CD₂, CH—F, CH—OH, and O; and
Y⁴ and Y⁵ are each independently selected from CH₂ and O;

provided that at most two of Y, Y³, Y⁴, and Y⁵ are O and that an Y being O is not bound to another Y also being O.

In a preferred embodiment, the present invention provides a compound of formula (I) as described herein, or a pharmaceutically acceptable salt thereof, wherein:
Y¹ and Y⁴ are each independently selected from CH₂ and O;
Y² is selected from CH₂ and CH—F; and
Y³ and Y⁵ are both CH₂.

In a particularly preferred embodiment, the present invention provides a compound of formula (I) as described herein, or a pharmaceutically acceptable salt thereof, wherein:
Y¹ and Y⁴ are both O; and
Y², Y³, and Y⁵ are all CH₂.

In one embodiment, the present invention provides a compound of formula (I) as described herein, or a pharmaceutically acceptable salt thereof, wherein:

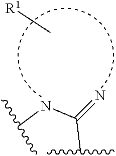

is selected from:

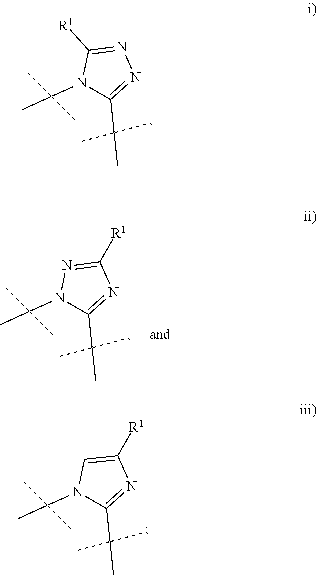

R¹ is selected from C₁-C₆-alkyl and R^{1a}R^{1b}NC(O)—;
R^{1a} is selected from hydroxy-C₁-C₆-alkyl and halo-hydroxy-C₁-C₆-alkyl;
R^{1b} is hydrogen;
R² is C₁-C₆-alkyl;
R³ is fluoro;
Y¹ and Y⁴ are each independently selected from O and CR⁴R⁵;
Y², Y³, and Y⁵ are each CR⁴R⁵;
R⁴ is selected from hydrogen and halogen; and
R⁵ is hydrogen.

In a preferred embodiment, the present invention provides a compound of formula (I) as described herein, or a pharmaceutically acceptable salt thereof, wherein:

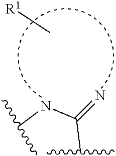

is selected from:

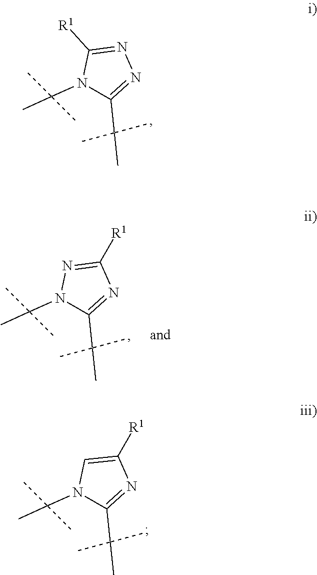

R¹ is selected from methyl and R^{1a}R^{1b}NC(O)—;
R^{1a} is selected from 2-hydroxy-2-methyl-propyl and 3,3,3-trifluoro-2-hydroxy-2-methyl-propyl;
R^{1b} is hydrogen;
R² is methyl;
R³ is fluoro;
Y¹ and Y⁴ are each independently selected from O and CR⁴R⁵;
Y², Y³, and Y⁵ are each CR⁴R⁵;
R⁴ is selected from hydrogen and fluoro; and R⁵ is hydrogen.

In one embodiment, the present invention provides a compound of formula (I) as described herein, or a pharmaceutically acceptable salt thereof, wherein said compound of formula (I) is selected from:
9-(2,6-difluorophenyl)-3-methyl-18-thia-2,4,5,8-tetrazatetracyclo[8.8.0.02,6.011,17]octadeca-1(10),3,5,8,11(17)-pentaene;
(7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaene;
(7S)-3-cyclopropyl-9-(2,6-difluorophenyl)-7-methyl-18-thia-2,4,5,8-tetrazatetracyclo[8.8.0.02,6.011,17]octadeca-1(10),3,5,8,11(17)-pentaene;
(7S)-9-(2,6-difluorophenyl)-7-methyl-3-pyridazin-3-yl-18-thia-2,4,5,8-tetrazatetracyclo[8.8.0.02,6.011,17]octadeca-1(10),3,5,8,11(17)-pentaene;
9-(2,6-difluorophenyl)-3-methyl-14-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-7-methyl-18-thia-2,4,5,8-tetrazatetracyclo[8.8.0.02,6.011,17]octadeca-1(10),5,8,11(17)-tetraen-3-one;
(8S)-10-(2,6-difluorophenyl)-8-methyl-19-thia-2,6,9-triazatetracyclo[9.8.0.02,7.012,18]nonadeca-1(11),3,6,9,12(18)-pentaen-5-one;
(7S)-9-(2,6-difluorophenyl)-15,15-difluoro-3,7-dimethyl-18-thia-2,4,5,8-tetrazatetracyclo[8.8.0.02,6.011,17]octadeca-1(10),3,5,8,11(17)-pentaene;
(7S,11RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-16-ol;
(7S,16RS)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,16R)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,16S)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,15RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-ol;
(7S,14S)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene;
(7S,14R)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene;
(7S,15R)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
(7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
9-(2,6-difluorophenyl)-3-methyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-4,7-dimethyl-18-thia-2,3,5,8-tetrazatetracyclo[8.8.0.02,6.011,17]octadeca-1(10),3,5,8,11(17)-pentaene;
(7S)-9-(2,6-difluorophenyl)-3,7-dimethyl-13,16-dioxa-18-thia-2,4,5,8-tetrazatetracyclo[8.8.0.02,6.011,17]octadeca-1(10),3,5,8,11(17)-pentaene;
azetidin-1-yl-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
azetidin-1-yl-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(3-fluoroazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-N-(2-fluoroethyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(8S)-10-(2,6-difluorophenyl)-4,8-dimethyl-19-thia-2,6,9-triazatetracyclo[9.8.0.02,7.012,18]nonadeca-1(11),3,6,9,12(18)-pentaen-5-one;
(7S,14R)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-ol;
(7S)-9-(2,6-difluorophenyl)-4,7-dimethyl-13,16-dioxa-18-thia-2,3,5,8-tetrazatetracyclo[8.8.0.02,6.011,17]octadeca-1(10),3,5,8,11(17)-pentaene;
(3-tert-butoxyazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S,15S)-9-(2,6-difluorophenyl)-3,7,15-trimethyl-13,16-dioxa-18-thia-2,4,5,8-tetrazatetracyclo[8.8.0.02,6.011,17]octadeca-1(10),3,5,8,11(17)-pentaene;
[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]-(3-ethoxyazetidin-1-yl)methanone;
(3-ethoxyazetidin-1-yl)-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-3,7-dimethyl-12-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-14,15-difluoro-3,7-dimethyl-18-thia-2,4,5,8-tetrazatetracyclo[8.8.0.02,6.011,17]octadeca-1(10),3,5,8,11(17)-pentaene;
(7S)-9-(2-chloro-6-fluoro-phenyl)-3,7-dimethyl-13,16-dioxa-18-thia-2,4,5,8-tetrazatetracyclo[8.8.0.02,6.011,17]octadeca-1(10),3,5,8,11(17)-pentaene;
(7S)—N-cyclopropyl-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-14,14,15,15-tetradeuterio-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S,15S)-9-(2,6-difluorophenyl)-4,7,15-trimethyl-13,16-dioxa-18-thia-2,3,5,8-tetrazatetracyclo[8.8.0.02,6.011,17]octadeca-1(10),3,5,8,11(17)-pentaene;
[(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]-(3-fluoroazetidin-1-yl)methanone;
(3-tert-butoxyazetidin-1-yl)-[(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-3-ethyl-7-methyl-13,16-dioxa-18-thia-2,4,5,8-tetrazatetracyclo[8.8.0.02,6.011,17]octadeca-1(10),3,5,8,11(17)-pentaene;
(7S)-3-cyclopropyl-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene;
(7S)-9-(2,6-difluorophenyl)-3-isopropyl-7-methyl-13,16-dioxa-18-thia-2,4,5,8-tetrazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-3-(difluoromethyl)-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene;
(7S,15S)-9-(2,6-difluorophenyl)-4-ethyl-7,15-dimethyl-13,16-dioxa-18-thia-2,3,5,8-tetrazatetracyclo[8.8.0.02,6.011,17]octadeca-1(10),3,5,8,11(17)-pentaene;
(7S)-9-(2,6-difluorophenyl)-3,7-dimethyl-13,16-dioxa-18-thia-2,5,8-triazatetracyclo[8.8.0.02,6.011,17]octadeca-1(10),3,5,8,11(17)-pentaene;
(7S,14R)-9-(2,6-difluorophenyl)-3,7-dimethyl-18-thia-2,5,8-triazatetracyclo[8.8.0.02,6.011,17]octadeca-1(10),3,5,8,11(17)-pentaen-14-ol;
(7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaene;
(7S,15S)-9-(2,6-difluorophenyl)-N-(2-hydroxyethyl)-7,15-dimethyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene;
(7S,14R)-9-(2,6-difluorophenyl)-14-fluoro-3,7-dimethyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-4,7-dimethyl-13-oxa-18-thia-2,3,5,8-tetrazatetracyclo[8.8.0.02,6.011,17]octadeca-1(10),3,5,8,11(17)-pentaene;
(7S)-9-(2,6-difluorophenyl)-4,7-dimethyl-13-oxa-18-thia-2,5,8-triazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2-chloro-6-fluoro-phenyl)-3,7-dimethyl-13-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-N-[(1-hydroxycyclopropyl)methyl]-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-(trans-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-(cis-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13,16-dioxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-(2-hydroxyethyl)-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-(2-hydroxy-2-methyl-propyl)-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide; and
(7S)-9-(2,6-difluorophenyl)-N-(2-hydroxy-2-methyl-propyl)-7-methyl-13-oxa-18-thia-2,3,5,8-tetrazatetracyclo[8.8.0.02,6.011,17]octadeca-1(10),3,5,8,11(17)-pentaene-4-carboxamide.

In a preferred embodiment, the present invention provides a compound of formula (I) as described herein, or a pharmaceutically acceptable salt thereof, wherein said compound of formula (I) is selected from:
(7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide; and
(7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide.

In a particularly preferred embodiment, the present invention provides a compound of formula (I) as described herein, or a pharmaceutically acceptable salt thereof, wherein said compound of formula (I) is (7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide.

In a particularly preferred embodiment, the present invention provides a compound of formula (I) as described herein, or a pharmaceutically acceptable salt thereof, wherein said compound of formula (I) is (7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene.

In a particularly preferred embodiment, the present invention provides a compound of formula (I) as described herein, or a pharmaceutically acceptable salt thereof, wherein said compound of formula (I) is (7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide.

In a particularly preferred embodiment, the present invention provides a compound of formula (I) as described herein, or a pharmaceutically acceptable salt thereof, wherein said compound of formula (I) is (7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide.

In a particularly preferred embodiment, the present invention provides a compound of formula (I) as described herein, or a pharmaceutically acceptable salt thereof, wherein said compound of formula (I) is (7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide.

In one embodiment, the present invention provides pharmaceutically acceptable salts of the compounds of formula (I) as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O ¹⁸O ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

Processes for the manufacture of the compound of formula (I) as described herein are also an object of the invention.

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before and in the claims, unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, NY. 2018). It is convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The preparation of compounds of formula (I) of the present invention may be carried out in sequential or convergent synthetic routes. Syntheses of the invention are shown in the following general schemes. The skills required for carrying out the reactions and purifications of the resulting products are known to those skilled in the art. The substituents and indices used in the following description of the processes have the significance given herein before unless indicated to the contrary.

In more detail, the compounds of formula (I) can be manufactured by the methods given below, by the methods given in the examples or by analogous methods. Appropriate reaction conditions for the individual reaction steps are known to a person skilled in the art. The reaction sequence is not limited to the one displayed in schemes 1-15, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The present compounds of formula (I) and their pharmaceutically acceptable salts may be prepared by a process described below (Schemes 1 to 15).

According to Scheme 1, a compound of formula (Ia) can be prepared from lactames of formula (II). Following thionation reaction using Lawesson's reagent or P₂S₅, lactames (II) are converted to corresponding thiolactames (III). Their reaction with hydrazides (IV) via a Pellizzari type process yields 1,2,4-triazoles of general formula (Ia). In alternative, 1,2,4-triazoles (Ia) can be obtained by reaction between thiolactames (II) and hydrazine to form hydrazones (V) followed by treatment with the corresponding trialkyl orthoacetate or acid chloride (VI). Furthermore, lactams (II) can be directly converted into 1,2,4-triazoles of formula (Ia) by treatment with bis(2-oxo-3-oxazolidinyl)phosphinic chloride, followed by reaction with hydrazides (IV) in presence of a strong base such as sodium hydride.

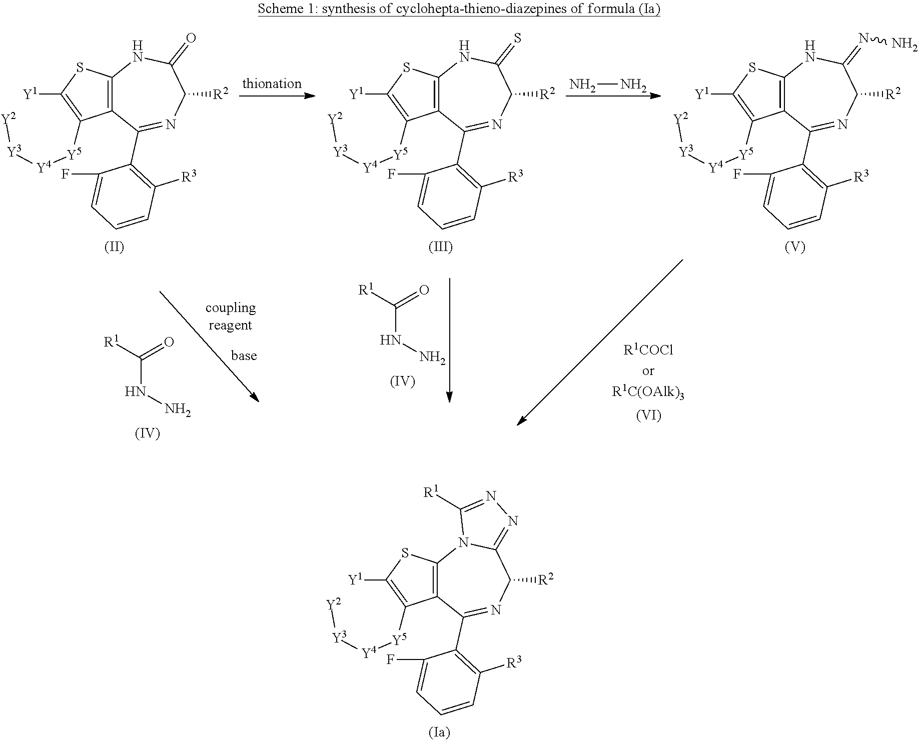

In certain embodiments of the invention, compounds of formula (Ib) can be prepared from lactams (II) by the process described below (Scheme 2). Electrophilic amination of lactams (II) using a suitable reagent such as O-(diphenylphosphinyl)hydroxylamine yields intermediates of formula (VII). Their final thermal cyclocondensation reaction with imidates (VIII) provides 1,2,4-triazoles (Ib).

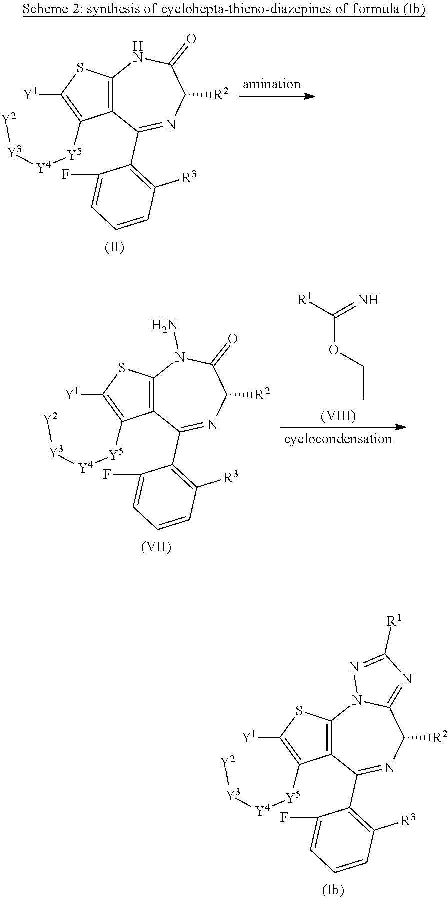

In certain embodiments of the invention where R¹ is an amide, compounds of formula (Ib) can be prepared according to Scheme 3. Esters (IX) are reacted with an amine (X) with or without addition of a suitable catalyst such as isopropylmagnesium chloride to form 1,2,4-triazole amides of formula (Ib). Furthermore, esters (IX) can be saponified to the corresponding acids (XI) under basic conditions, for instance by treatment with an aqueous or alcoholic solution of sodium hydroxide or lithium hydroxide. Finally, 1,2,4-triazole amides (Ib) are obtained by standard amide coupling reaction between acids (XI) and amines HNR⁴R⁵ (X).

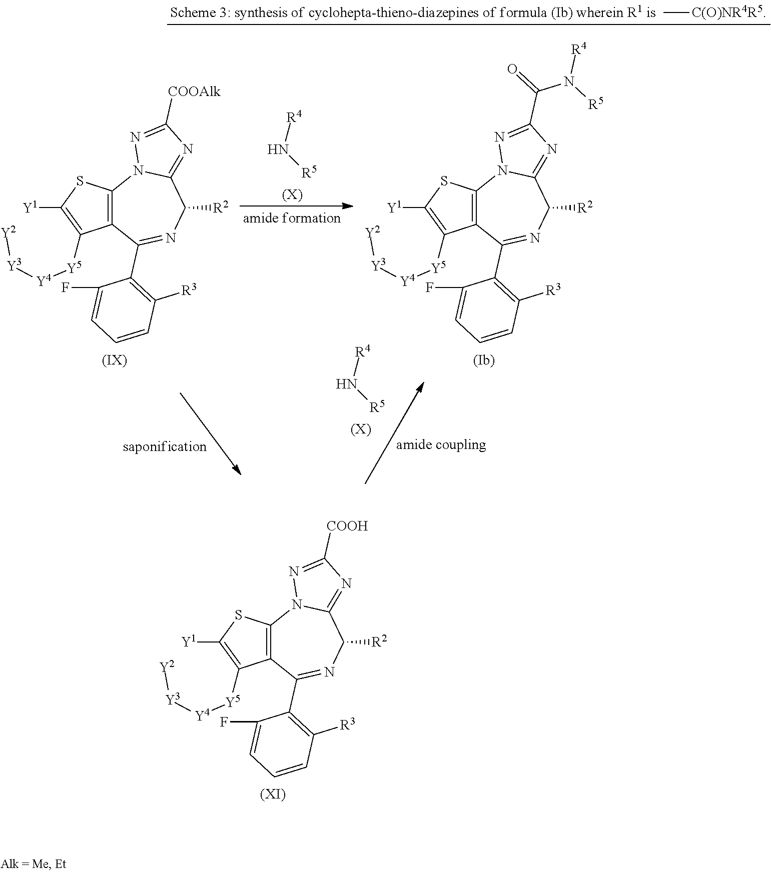

In certain embodiments of the invention compounds of formula (Ic) can be obtained in two steps according to the process described in Scheme 4. It is widely accepted that 3-hydroxy-1,2,4-triazoles are existing as two tautomeric forms and in this invention they will be represented exclusively in their most stable form (triazolones). To this end, hydrazones (V) can be reacted with 1,1′-carbonyldiimidazole (CDI) to yield triazolones of formula (Ic) (Scheme 4).

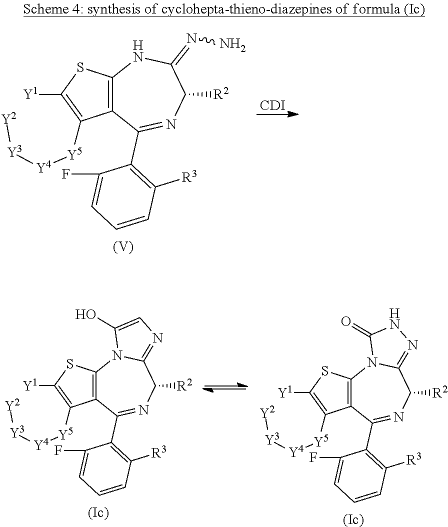

In further embodiments of the invention, imidazoles of formula (Id) can be prepared as described below (Scheme 5). Thiolactams (III) are reacted with ammonia to form amidines of formula (XII). Following a reaction with propargylamine under acid catalysis, amidines (XII) can be converted to methyl imidazoles (Id).

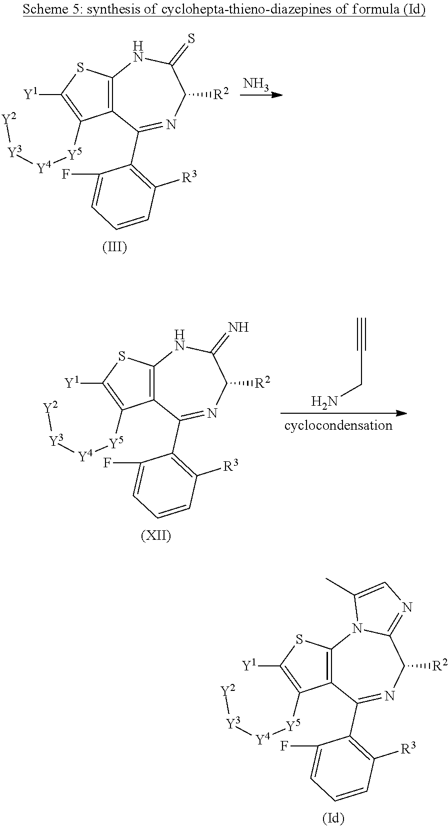

In further embodiments of the invention, imidazoles of formula (Ie) can be prepared as described below (Scheme 6). Lactams (II) are reacted with amino alcohols (XIII) following activation with bis(2-oxo-3-oxazolidinyl)phosphinic chloride (BOP—Cl) in presence of a base (NaH). Primary alcohols (XIV) are then cyclized under oxidative conditions to provide imidazoles of formula (Ie).

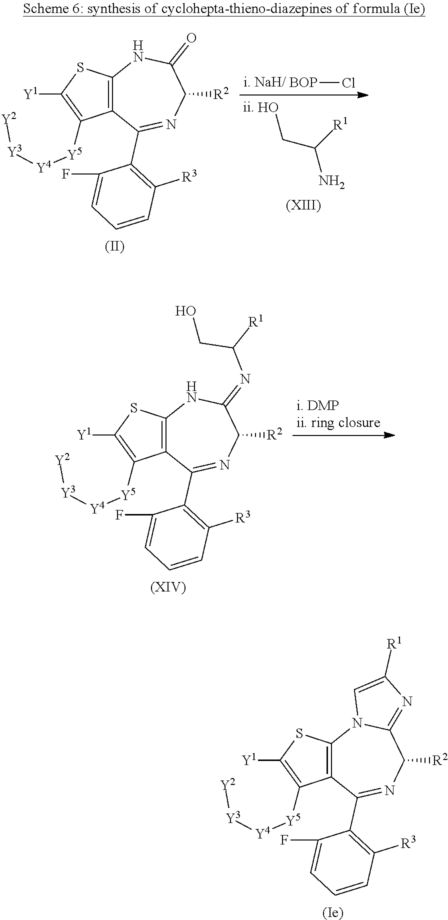

In further embodiments of the invention, compounds of formula (If) can be prepared as described below (Scheme 7). Amidines (XII) are reacted with ethyl propiolate to form pyrimido[1,2-a][1,4]benzodiazepin-3-ones (XV). Following a reaction with a brominating agent such as N-bromosuccinimide (NBS) and subsequent Suzuki-reaction with trimethylboroxine, compounds of formula (If) can be obtained.

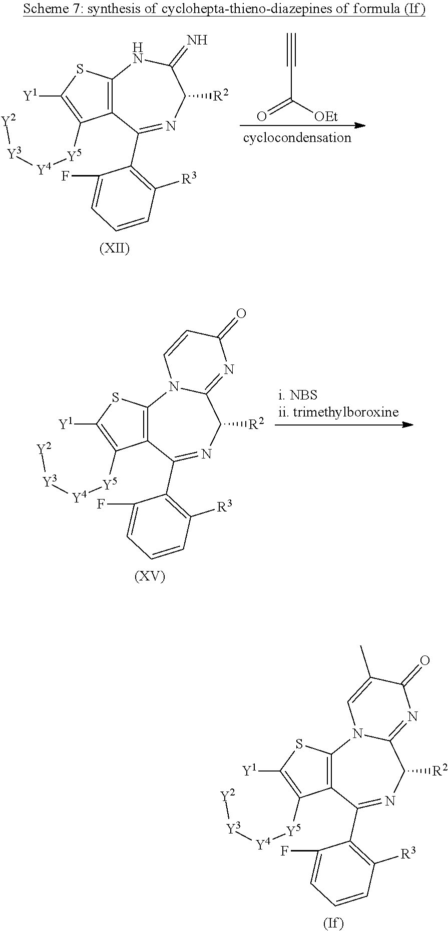

In further embodiments of the invention, fluorinated compounds of formula (I) can be prepared as described below (Scheme 8). Cyclohepta-thieno-diazepines (I) are regioselectively oxidized with potassium peroxodisulphate to provide ketones (XVI), which, in turn, can be reduced by treatment with lithium or potassium borohydride to give benzylic alcohols (XVII). Following a reaction with a fluorinating agent such as diaminosulfur trifluoride (DAST) or Deoxo-Fluor®, monofluorinated derivatives of formula (I) can be obtained. Resulting diastereomers can be separated by chromatography.

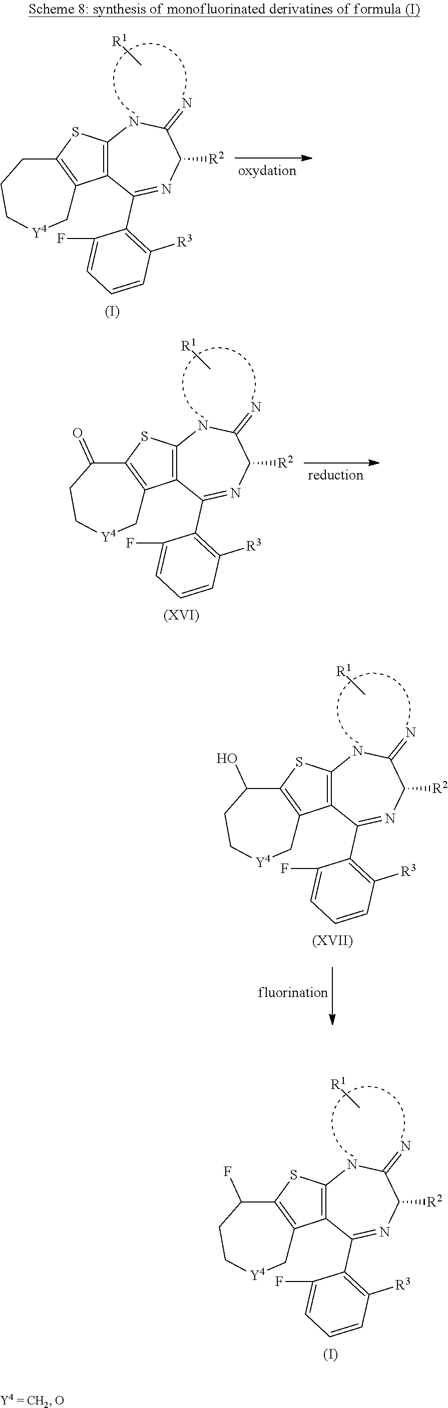

In further embodiments of the invention, geminal difluorinated derivatives of formula (I) and monofluorinated derivatives of formula (I) can be prepared as described below (Scheme 9). Dioxolanes (XVIII) are deprotected by exposure to acids such as acetic acid to provide ketones (XIX). Their reduction with lithium or potassium borohydride affords secondary alcohols (XX). Treatment of intermediates (XIX) or (XX) with a fluorinating agent such as diaminosulfur trifluoride (DAST) or Deoxo-Fluor® affords geminal difluorinated derivatives (I) or monofluorinated derivatives (I), respectively. Resulting diastereomers can be separated by chromatography.

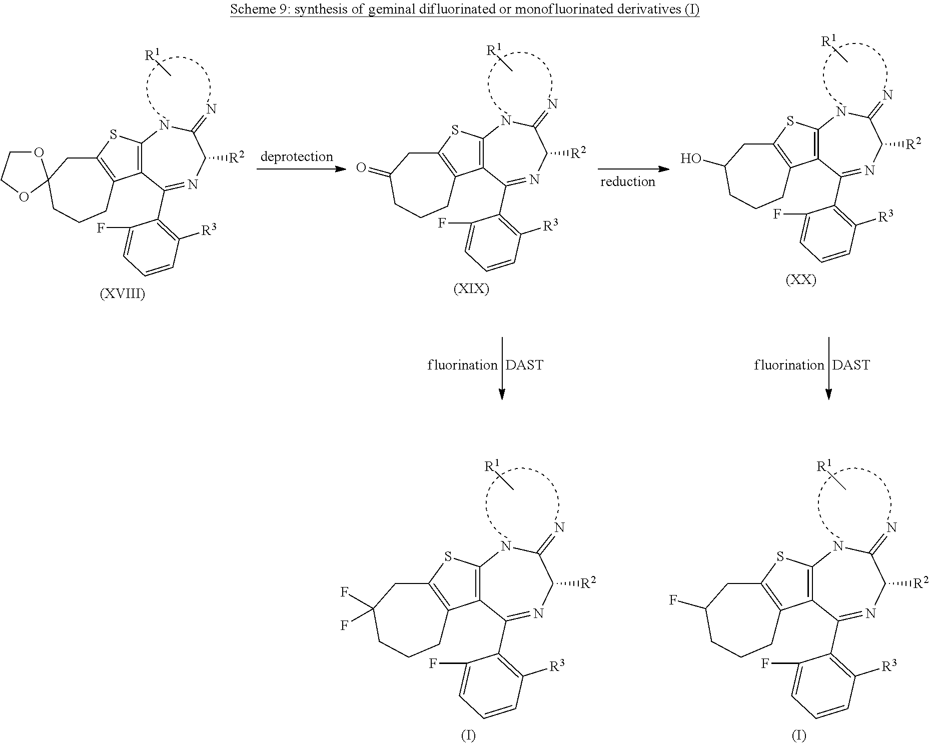

In further embodiments of the invention, germinal difluorinated derivatives of formula (I) and monofluorinated derivatives of formula (I) can be prepared as described below (Scheme 10). Dioxolanes (XXI) are deprotected by exposure to acids such as acetic acid to provide ketones (XXII). Their reduction with lithium or potassium borohydride affords secondary alcohols (XXIII). Treatment of intermediates (XXII) or (XXIII) with a fluorinating agent such as diaminosulfur trifluoride (DAST) or Deoxo-Fluor® affords geminal difluorinated derivatives (I) or monofluorinated derivatives (I), respectively. Resulting diastereomers can be separated by chromatography.

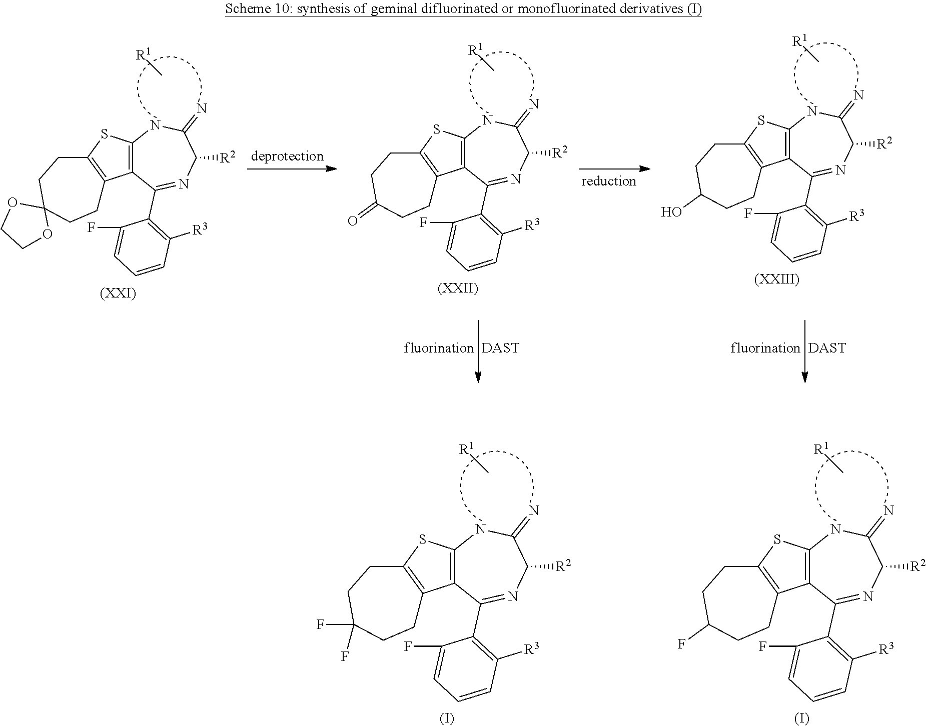

In further embodiments of the invention, vicinal difluorinated derivatives of formula (I) can be prepared as described below (Scheme 11). Alkenes (XXIV) are treated with a mixture of N-bromosuccinimide and Olah's reagent (py.HF), followed by silver(I) fluoride (AgF) to give vicinal difluorinated derivatives (I) as a mixture of diastereomers, that can be separated by chromatography.

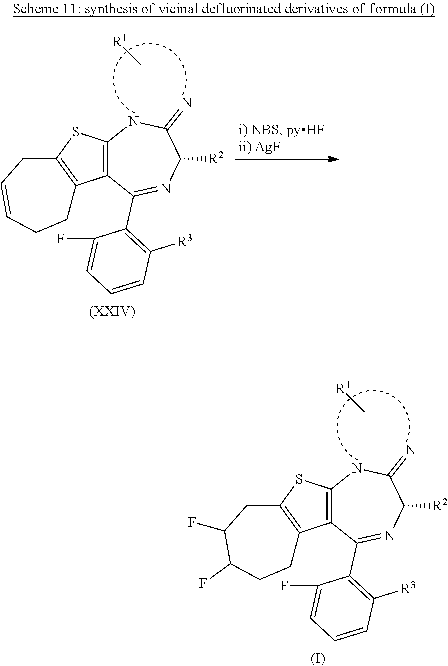

In further embodiments of the invention, monofluorinated oxepines of formula (I) can be prepared as described below (Scheme 12). Benzyl ethers (XXV) are deprotected by hydrogenation or treatment with boron tribromide to give secondary alcohols (XXVI). Fluorination of this intermediates (XXII) with a fluorinating agent such as diaminosulfur trifluoride (DAST) or Deoxo-Fluor® affords monofluorinated oxepines of formula (I). Resulting diastereomers can be separated by chromatography.

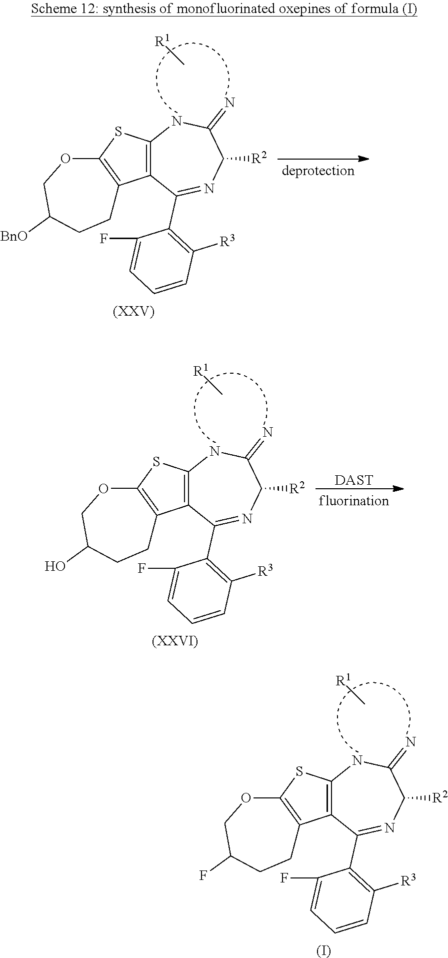

The synthesis of the lactams (II) and their precursors is highlighted in the following schemes.

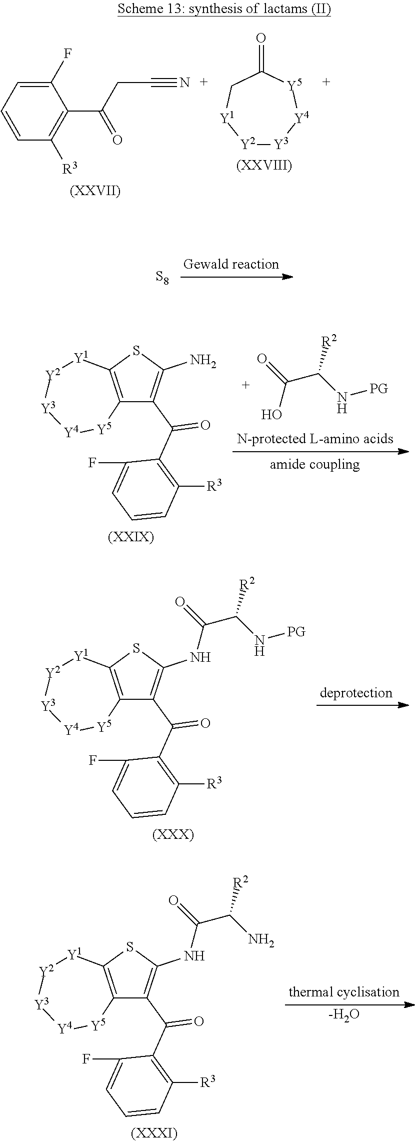

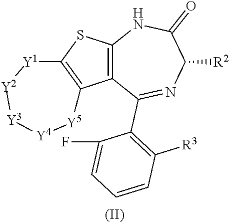

Lactames (II) can be prepared according to a process described in Scheme 13. Commercially available nitriles (XXVII) are reacted with ketones (XXVIII) in presence of an organic base and sulfur (Gewald reaction) to yield 2-amino-thiophenes of formula (XXIX). Notably, in the Gewald reaction, a mixture of regioisomers is formed (from 20:1 to 1:1) depending on the presence of heteroatoms in the 7-membered ring and substituents. Conveniently, the required regioisomer can be obtained pure after chromatographic removal of the undesired minor isomer and this can be performed at any stage of the synthesis. Compounds of formula (XXX) can be prepared by an amide coupling reaction between 2-amino-thiophenes (XXIX) and N-Boc or N-Cbz protected L-amino acids upon activation with phosphoryl chloride (POCl₃), or by other methods known to those skilled in the art. Removal of N-Boc or N-Cbz protecting group can be effected with mineral acids (e.g. HCl) or organic acids (e.g. trifluoroacetic acid) to yield amines of formula (XXXI). In case of N-Cbz protected intermediates (XXX), the deprotection reaction can be accomplished in addition by hydrogenation or using iodotrimethylsilane. Final intramolecular condensation reaction promoted by acidic media (e.g. silica or acetic acid) and heat (80-110° C.) provides desired lactam building block of formula (II).

In alternative, 2-aminothiophenes (XXIX) can be synthesized by the process described in Scheme 14. Ketones (XXVIII) are converted into □-chlorovilyl aldehydes (XXXII) via a Vilsmeier-Haack-Arnold reaction upon treatment with POCl₃ in DMF. Subsequent reaction between □-chlorovilyl aldehydes (XXXII) and potassium thiocyanate resulted in the formation of □-thiocyanato aldehydes (XXXIII). Reaction of (XXXIII) with nitromethane in presence of a base (DIPEA) provides 2-nitrothiophenes (XXXIV) which, in turns, can be reduced with iron powder and acetic anhydride under acidic conditions to provide 2-acetylamino thiophenes (XXXV). Their final Friedel Crafts reaction with acyl chlorides (XXXVI) to form intermediates (XXXVII), followed by deacetylation reaction by treatment with K₂CO₃ yields desired 2-aminothiophene derivatives of formula (XXIX).

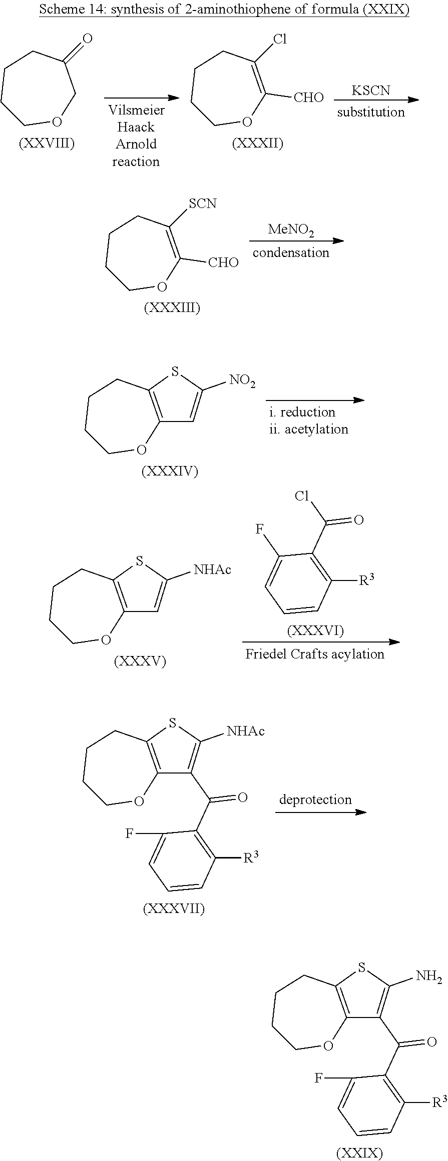

In alternative, 2-aminothiophenes (XXIX) can be synthesized by the following process described in Scheme 15. □,□-unsaturated ketones (XXXVIII) are converted to silyl enol ether (XXXIX) using triethylsilane and rhodium acetate as catalyst. Following electrophilic bromination with N-bromosuccinimide (NBS) they form □-bromo ketones of formula (XXXX). Nucleophilic substitution (S_{N}2) reaction between intermediates (XXXX) and potassium thioacetate forms ketones (XXXXI) which, in turns, can be cyclized with nitriles (XXVII) to yield 2-aminothiophene derivatives of general formula (XXIX).

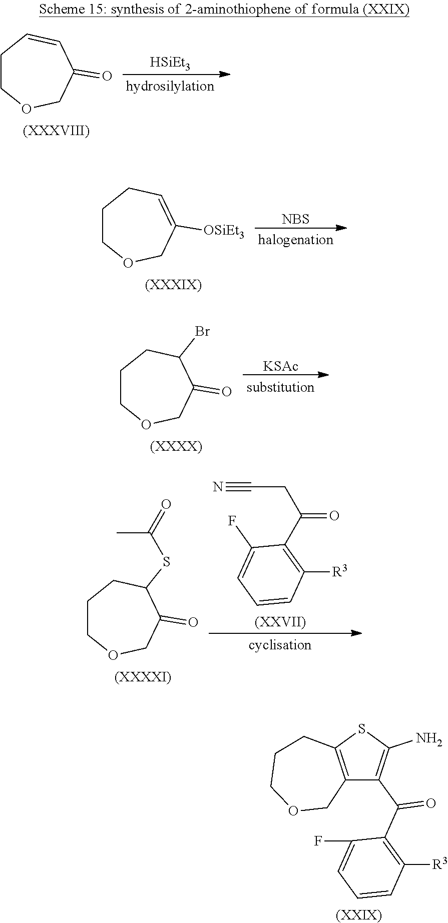

Notably, in the processes described in Scheme 1 to 15, racemization at the chiral center occurs to various extents (20-100%), depending on specific reaction conditions adopted. As a result, chiral purification (e.g. by HPLC or SFC) of final derivatives of formula (I), is required to obtain single enantiomers (enantiomeric excess (ee) above 97%).

In one aspect, the present invention provides a process of manufacturing the compounds of formula (I) described herein, wherein said process is as described in any one of Schemes 1 to 15 above.

In a further aspect, the present invention provides a compound of formula (I) as described herein, or a pharmaceutically acceptable salt thereof, when manufactured according to the processes disclosed herein.

### Using the Compounds of the Invention

As explained in the background section and illustrated in the experimental section, the compounds of formula (I) and their pharmaceutically acceptable salts possess valuable pharmacological properties that make them useful for the treatment or prevention of diseases or condictions that are associated with the GABAA γ1 receptor.

In one aspect, the present invention provides a compound of formula (I) as described herein, or a pharmaceutically acceptable salt thereof, for use as therapeutically active substance.

In a further aspect, the present invention provides a method for treating or preventing acute neurological disorders, chronic neurological disorders and/or cognitive disorders in a subject, said method comprising administering an effective amount of a compound of formula (I) as described herein, or a pharmaceutically acceptable salt thereof, or a pharmaceutical composition described herein, to the subject.

In a further aspect, the present invention provides the use of a compound of formula (I) as described herein, or a pharmaceutically acceptable salt thereof, or a pharmaceutical composition described herein, in a method for treating or preventing acute neurological disorders, chronic neurological disorders and/or cognitive disorders in a subject.

In a further aspect, the present invention provides a compound of formula (I) as described herein, or a pharmaceutically acceptable salt thereof, or a pharmaceutical composition described herein, for use in a method for treating or preventing acute neurological disorders, chronic neurological disorders and/or cognitive disorders in a subject.

In a further aspect, the present invention provides the use of a compound of formula (I) as described herein, or a pharmaceutically acceptable salt thereof, for the manufacture of a medicament for the treatment of prevention of acute neurological disorders, chronic neurological disorders and/or cognitive disorders.

In one embodiment, said acute neurological disorders, chronic neurological disorders and/or cognitive disorders are selected from autism spectrum disorders (ASD), Angelman syndrome, age-related cognitive decline, Rett syndrome, Prader-Willi syndrome, amyotrophic lateral sclerosis (ALS), fragile-X disorder, negative and/or cognitive symptoms associated with schizophrenia, tardive dyskinesia, anxiety, social anxiety disorder (social phobia), panic disorder, agoraphobia, generalized anxiety disorder, disruptive, impulse-control and conduct disorders, Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurode generative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar disorders, epilepsy and pain.

In one embodiment, said acute neurological disorders, chronic neurological disorders and/or cognitive disorders are selected from Alzheimer's disease, mild cognitive impairment (MCI), age-related cognitive decline, negative and/or cognitive symptoms associated with schizophrenia, bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder.

In a preferred embodiment, said acute neurological disorders, chronic neurological disorders and/or cognitive disorders are selected from autism spectrum disorder (ASD), Angelman syndrome, Alzheimer's disease, negative and/or cognitive symptoms associated with schizophrenia and post-traumatic stress disorder (PTSD).

In a preferred embodiment, said acute neurological disorders, chronic neurological disorders and/or cognitive disorders are selected from autism spectrum disorder (ASD), Rett syndrome, post-traumatic stress disorder and fragile-X disorder.

In a preferred embodiment, said acute neurological disorders, chronic neurological disorders and/or cognitive disorders are selected from autism spectrum disorder (ASD) and Angelman syndrome.

In a particularly preferred embodiment, said acute neurological disorders, chronic neurological disorders and/or cognitive disorders are autism spectrum disorder (ASD).

In a particularly preferred embodiment, said acute neurological disorders, chronic neurological disorders and/or cognitive disorders are Angelman syndrome.

In a further particularly preferred embodiment, said acute neurological disorders, chronic neurological disorders and/or cognitive disorders are autism spectrum disorder (ASD), targeting core symptoms and associated comorbidities, such as anxiety and irritability, social anxiety disorder (social phobia) and generalized anxiety disorder.

### Pharmaceutical Compositions and Administration

In one aspect, the present invention provides pharmaceutical compositions comprising compounds of formula (I) or their pharmaceutically acceptable salts as defined herein and one or more pharmaceutically acceptable excipients. Exemplary pharmaceutical compositions are described in the Example section below.

In a further aspect, the present invention relates to pharmaceutical compositions comprising compounds of formula (I) or their pharmaceutically acceptable salts as defined above and one or more pharmaceutically acceptable excipients for the treatment or prevention of acute neurological disorders, chronic neurological disorders and/or cognitive disorders.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

Building blocks A to T can be produced according to the following synthetic procedures.

### Building Block A

### 3-(2,6-difluorophenyl)-9-thia-4,7-diazatricyclo[8.5.0.02,8]pentadeca-1(10),2(8),3-trien-6-one

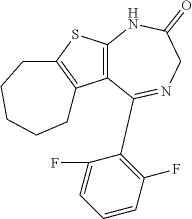 [SMILES: O=C1CN=C(c2c(F)cccc2F)c2c(sc3c2CCCCC3)N1]

### a) (2-amino-5,6,7,8-tetrahydro-4H-cyclohepta[b]thiophen-3-yl)-(2,6-difluorophenyl)methanone

To a solution of 3-(2,6-difluorophenyl)-3-oxopropanenitrile (CAS 40017-76-3, 3 g, 16.6 mmol) in ethanol (182 mL) was added cycloheptanone (1.86 g, 1.95 mL, 16.6 mmol), morpholine (1.44 g, 1.44 mL, 16.6 mmol) and sulfur (531 mg, 16.6 mmol). The reaction suspension was heated to 90° C. and stirred for 40 h. After cooling, the reaction mixture was concentrated in vacuo and the residue (dark brown oil) was purified by flash column chromatography (silica, heptane/ethyl acetate 100:0 to 77:23) to obtain the title compound (1.91 g, 32%) as a yellow solid. MS m/z: 308.1 [M+H]⁺, ESI pos.

### b) tert-butyl (2-((3-(2,6-difluorobenzoyl)-5,6,7,8-tetrahydro-4H-cyclohepta[b]thiophen-2-yl)amino)-2-oxoethyl)carbamate

To a solution of (2-amino-5,6,7,8-tetrahydro-4H-cyclohepta[b]thiophen-3-yl)(2,6-difluorophenyl)-methanone (1.78 g, 5.79 mmol) in dichloromethane (40 mL), Boc-glycine (1.12 g, 6.37 mmol) was added. The dark-yellow solution was cooled to 0-4° C. (ice-bath), and pyridine (2.29 g, 2.34 mL, 29 mmol) was added, followed by phosphoryl chloride (1.15 g, 0.702 ml, 7.53 mmol). The mixture was stirred for 60 min at 0-5° C. Then, it was poured into a stirred mixture of ethyl acetate (160 mL), ice-water (100 mL) and saturated aqueous sodium hydrogencarbonate solution (60 mL), and vigorously stirred for 20 min. The aqueous layer was separated and back-extracted with ethyl acetate (100 mL). The combined organic layers were washed with brine (150 mL), dried (Na₂SO₄) and concentrated in vacuo. The residue was purified by flash column chromatography (silica, heptane/ethyl acetate 9:1 to 1:9) to afford the title compound (2.43 g, 89%) as a yellow foam. MS m/z: 365.2 [M−Boc+H]⁺, ESI pos.

### c) 2-amino-N-[3-(2,6-difluorobenzoyl)-5,6,7,8-tetrahydro-4H-cyclohepta[b]thiophen-2-yl]acetamide

To a solution of tert-butyl (2-((3-(2,6-difluorobenzoyl)-5,6,7,8-tetrahydro-4H-cyclohepta[b]thiophen-2-yl)amino)-2-oxoethyl)carbamate (2.43 g, 5.23 mmol) in dichloromethane (50 mL), trifluoroacetic acid (11.9 g, 8.06 mL, 105 mmol) was added carefully. The resulting brownish reaction solution was stirred for 23 h at room temperature. The reaction mixture was concentrated in vacuo at 45° C. The residue was dissolved in methanol (20 mL), triethylamine (10 mL) was added and the mixture was stirred for 10 min at room temperature. The mixture was concentrated in vacuo again and the crude product was purified by flash column chromatography (silica, dichloromethane/2.0 M ammonia in methanol, 100:0 to 98:2) to afford the title compound (0.96 g, 50%) as a yellow solid. MS m/z: 365.1 [M+H]⁺, ESI pos.

### d) 3-(2,6-difluorophenyl)-9-thia-4,7-diazatricyclo[8.5.0.02,8]pentadeca-1(10),2(8),3-trien-6-one

To a solution of 2-amino-N-(3-(2,6-difluorobenzoyl)-5,6,7,8-tetrahydro-4H-cyclohepta[b]thiophen-2-yl)acetamide (1.01 g, 2.77 mmol) in toluene (55 mL) was added silica 60-200 μm (4.0 g), and the suspension was stirred for 65 h at 90° C. After cooling, the suspension was filtered on sintered funnel, washed with ethyl acetate (3×50 mL), and the filtrate was concentrated in vacuo to afford the title compound (932 mg, 95%) as a light brown solid, which was used in the next step without further purification. MS m/z: 347.1 [M+H]⁺, ESI pos.

### Building Block B

### 3-(2,6-difluorophenyl)-9-thia-4,7-diazatricyclo[8.5.0.02,8]pentadeca-1(10),2(8),3-triene-6-thione

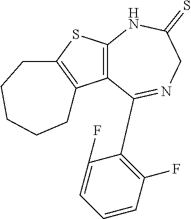 [SMILES: Fc1cccc(F)c1C1=NCC(=S)Nc2sc3c(c21)CCCCC3]

In a 5 mL microwave vial, 3-(2,6-difluorophenyl)-9-thia-4,7-diazatricyclo[8.5.0.02,8]pentadeca-1(10),2(8),3-trien-6-one (Building block A, 190 mg, 0.549 mmol) was suspended in tetrahydrofuran (2.5 mL) and Lawesson's reagent (133 mg, 0.329 mmol) was added. The vial was sealed and the mixture was heated for 30 min at 100° C. in a microwave reactor. After cooling, the reaction mixture was absorbed on diatomaceous earth material Isolute HM-N (˜6-8 g) and concentrated in vacuo. The residue was purified by flash column chromatography (Isolute Flash NH₂, heptane/ethyl acetate 9:1 to 0:1, followed by ethyl acetate/methanol 10:1), followed by trituration in tert-butyl methyl ether/heptane (1:1 (v/v), 5 mL), filtration, washing with tert-butyl methyl ether/heptane (1:1 (v/v), 2×2.5 mL) and drying in vacuo to afford the title compound (130 mg, 65%) as a light brown solid. MS m/z: 363.1 [M+H]⁺, ESI pos.

### Building Block C

### (5S)-3-(2,6-difluorophenyl)-5-methyl-9-thia-4,7-diazatricyclo[8.5.0.02,8]pentadeca-1(10),2(8),3-trien-6-one

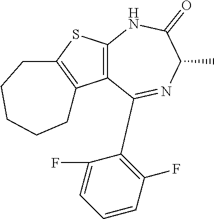 [SMILES: C[C@@H]1N=C(c2c(F)cccc2F)c2c(sc3c2CCCCC3)NC1=O]

### a) tert-butyl N-[(1S)-2-[[3-(2,6-difluorobenzoyl)-5,6,7,8-tetrahydro-4H-cyclohepta[b]thiophen-2-yl]amino]-1-methyl-2-oxo-ethyl]carbamate

To a solution of (tert-butoxycarbonyl)-L-alanine (1.25 g, 6.59 mmol) in N,N-dimethylformamide (5 mL) was added DIPEA (2.56 g, 3.45 mL, 19.8 mmol), followed by (1-[bis(dimethylamino)methylene]-1H-1,2,3-triazolo[4,5-b]pyridinium 3-oxide hexafluorophosphate (HATU, 3.26 g, 8.57 mmol), resulting in a light yellow solution. After stirring for 5 min, (2-amino-5,6,7,8-tetrahydro-4H-cyclohepta[b]thiophen-3-yl)(2,6-difluorophenyl)methanone (1.62 g, 5.27 mmol) was added. The mixture was stirred for 1 h at room temperature, followed by 48 h at 55° C. After cooling, the mixture was diluted with water (20 mL) and ethyl acetate (30 mL). The aqueous layer was extracted with ethyl acetate (2×30 mL). The combined organic layers were washed with water (30 mL) and brine (30 mL), dried (Na₂SO₄), filtered, and concentrated in vacuo. The crude product was purified by flash chromatography (silica, heptane/ethyl acetate, 100:0 to 70:30) to afford the title compound (1.75 g, 53%) as a yellow solid. MS m/z: 423.3 [M−CH₂=CMe₂+H]⁺, ESI pos.

### b) (2S)-2-amino-N-[3-(2,6-difluorobenzoyl)-5,6,7,8-tetrahydro-4H-cyclohepta[b]thiophen-2-yl]propanamide

A solution of tert-butyl N-[(1S)-2-[[3-(2,6-difluorobenzoyl)-5,6,7,8-tetrahydro-4H-cyclohepta[b]thiophen-2-yl]amino]-1-methyl-2-oxo-ethyl]carbamate (1.75 g, 3.51 mmol) in dichloromethane (100 mL) was cooled to 0-4° C. (ice bath) and trifluoroacetic acid (8.34 g, 5.63 mL, 73.1 mmol) was added carefully. The yellow reaction mixture was stirred for 20 h at 23° C., then concentrated in vacuo. The residue was dissolved in dichloromethane (20 mL) and saturated aqueous sodium hydrogen carbonate (20 mL). The aqueous phase was extracted with dichloromethane (2×20 mL). The combined organic layers were dried (Na₂SO₄), filtered and concentrated in vacuo to afford the title compound (1.29 g, 92%) as a yellow solid, which was used in the next step without further purification MS m/z: 379.1 [M+H]⁺, ESI pos.

### c) (5S)-3-(2,6-difluorophenyl)-5-methyl-9-thia-4,7-diazatricyclo[8.5.0.02,8]pentadeca-1(10),2(8),3-trien-6-one

A mixture of (2S)-2-amino-N-(3-(2,6-difluorobenzoyl)-5,6,7,8-tetrahydro-4H-cyclohepta[b]thiophen-2-yl)propanamide (1.29 g, 3.41 mmol) in toluene (30 mL) was heated to 60° C., then silica gel (60-200 □m, 8 g) was added and the resulting yellow suspension was stirred for 20 h at 90° C. After cooling to ca. 40° C., the suspension was filtered, washed with ethyl acetate, and the filtrate was concentrated in vacuo. The residue was purified by flash column chromatography (silica, heptane/ethyl acetate, 1:0 to 1:1) to afford the title compound (1.21 g, 96%) as a yellow solid. MS m/z: 361.4 [M+H]⁺, ESI pos.

### Building Block D

### (5S)-3-(2,6-difluorophenyl)-5-methyl-9-thia-4,7-diazatricyclo[8.5.0.02,8]pentadeca-1(10),2(8),3-triene-6-thione

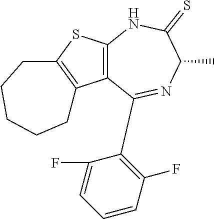 [SMILES: C[C@@H]1N=C(c2c(F)cccc2F)c2c(sc3c2CCCCC3)NC1=S]

To a solution of (5S)-3-(2,6-difluorophenyl)-5-methyl-9-thia-4,7-diazatricyclo[8.5.0.02,8]pentadeca-1(10),2(8),3-trien-6-one (Building block C, 310 mg, 0.860 mmol) in tetrahydrofuran (10 mL) was added Lawesson's reagent (209 mg, 0.516 mmol) and the mixture was heated in the microwave for 30 min at 100° C. After cooling, the reaction mixture was concentrated in vacuo. The residue was purified by flash column chromatography (silica, 0-100% dichloromethane in heptane; then 0-10% methanol in dichloromethane) to afford the title compound (274 mg, 81%) as a yellow solid. MS m/z: 377.1 [M+H]⁺, ESI pos.

### Building Block E

### (5S)-3-(2,6-difluorophenyl)-12,12-difluoro-5-methyl-9-thia-4,7-diazatricyclo[8.5.0.02,8]pentadeca-1(10),2(8),3-triene-6-thione

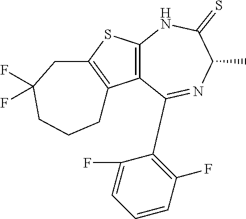 [SMILES: C[C@@H]1N=C(c2c(F)cccc2F)c2c(sc3c2CCCC(F)(F)C3)NC1=S]

### a) (2-amino-7,7-difluoro-5,6,7,8-tetrahydro-4H-cyclohepta[b]thiophen-3-yl)(2,6-difluoro-phenyl)methanone

To a solution of 3-(2,6-difluorophenyl)-3-oxopropanenitrile (1086 mg, 6.0 mmol) in anhydrous ethanol (60 mL) at room temperature, was added 4,4-difluorocycloheptan-1-one (0.89 g, 6.0 mmol), sulfur (192 mg, 6 mmol) and morpholine (522 mg, 0.522 mL, 6 mmol). The mixture was stirred at 80° C. for 16 h. The dark brown reaction solution was concentrated in vacuo. The residue was purified by flash column chromatography (silica, 0-30% ethyl acetate in heptane) to afford the title compound (138 mg, 7%) as a light yellow foam (MS m/z: 344.1 [M+H]⁺, ESI pos.) and its regioisomer (2-amino-6,6-difluoro-5,6,7,8-tetrahydro-4H-cyclohepta[b]thiophen-3-yl)(2,6-difluorophenyl)methanone (1.51 g, 80% purity by HPLC, 59%) as a yellow solid (MS m/z: 344.1 [M+H]⁺, ESI pos.).

### b) tert-butyl N-[(1S)-2-[[3-(2,6-difluorobenzoyl)-7,7-difluoro-4,5,6,8-tetrahydrocyclohepta[b]thiophen-2-yl]amino]-1-methyl-2-oxo-ethyl]carbamate

To a solution of (tert-butoxycarbonyl)-L-alanine (555 mg, 2.93 mmol) in N,N-dimethylformamide (5 mL) was added DIPEA (1.14 g, 1.54 mL, 8.8 mmol), followed by HATU (1.45 g, 3.81 mmol). The resulting light yellow solution was stirred for 5 min, before addition of (2-amino-7,7-difluoro-5,6,7,8-tetrahydro-4H-cyclohepta[b]thiophen-3-yl)(2,6-difluorophenyl)methanone (806 mg, 2.35 mmol). The reaction mixture was stirred at 60° C. for 4 days. The brown reaction solution was diluted with water (20 mL) and ethyl acetate (20 mL). The aqueous layer was extracted with ethyl acetate (2×20 mL). The combined organic layers were washed with water (20 mL) and brine (20 mL), dried (Na₂SO₄), filtered and concentrated in vacuo. The residue was purified by flash column chromatography (silica, 0-30% ethyl acetate in heptane) to afford the title compound (121 mg, 8%) as a yellow solid. MS m/z: 513.1 [M−H]⁻, ESI neg.

### c) (2S)-2-amino-N-[3-(2,6-difluorobenzoyl)-7,7-difluoro-4,5,6,8-tetrahydrocyclohepta[b]thiophen-2-yl]propanamide

To a solution of tert-butyl N-[(1S)-2-[[3-(2,6-difluorobenzoyl)-7,7-difluoro-4,5,6,8-tetrahydrocyclohepta[b]thiophen-2-yl]amino]-1-methyl-2-oxo-ethyl]carbamate (265 mg, 0.515 mmol) in dichloromethane (10 mL) was added under ice cooling trifluoroacetic acid (1.17 g, 0.794 mL, 10.3 mmol). The orange reaction solution was stirred at 23° C. for 16 h. The reaction mixture was concentrated in vacuo and extracted with dichloromethane and aqeuous sodium hydrogen carbonate. The combined organic layers were dried (Na₂SO₄) and concentrated in vacuo to afford the title compound (246 mg, quant.) as a yellow solid. MS m/z: 415.2 [M+H]⁺, ESI pos.

### d) (5S)-3-(2,6-difluorophenyl)-12,12-difluoro-5-methyl-9-thia-4,7-diazatricyclo[8.5.0.02,8]pentadeca-1(10),2(8),3-triene-6-one

To a solution of (2S)-2-amino-N-[3-(2,6-difluorobenzoyl)-7,7-difluoro-4,5,6,8-tetrahydrocyclohepta[b]thiophen-2-yl]propanamide (246 mg, 0.59 mmol) in toluene (10 mL) at 60° C. was added silica gel (60-200 μm, 1.6 g). The yellow suspension was stirred at 90° C. for 20 h, before being cooled to 40° C. The mixture was filtered directly through a sintered funnel and the filter cake was rinsed with ethyl acetate. The filtrate was concentrated in vacuo to afford the title compound (256 mg, quant.) as a yellow solid. The crude was used in the following step without further purification. MS m/z: 397.2 [M+H]⁺, ESI pos.

### e) (5S)-3-(2,6-difluorophenyl)-12,12-difluoro-5-methyl-9-thia-4,7-diazatricyclo[8.5.0.02,8]pentadeca-1(10),2(8),3-triene-6-thione

To a solution of (5S)-3-(2,6-difluorophenyl)-12,12-difluoro-5-methyl-9-thia-4,7-diazatricyclo[8.5.0.02,8]pentadeca-1(10),2(8),3-triene-6-one (256 mg, 0.594 mmol) in tetrahydrofuran (10 mL) was added Lawesson's reagent (157 mg, 0.387 mmol). The reaction mixture was heated in the microwave at 100° C. for 30 min, then concentrated in vacuo. The residue was purified by flash column chromatography (silica, 0-50% ethyl acetate in heptane) to afford the title compound (192 mg, 78%) as a yellow solid. MS m/z: 413.1 [M+H]⁺, ESI pos.

### Building Block F

### 3-(2,6-difluorophenyl)-13-oxa-9-thia-4,7-diazatricyclo[8.5.0.02,8]pentadeca-1(10),2(8),3-triene-6-thione

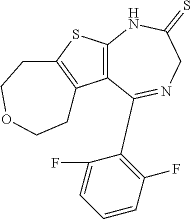 [SMILES: Fc1cccc(F)c1C1=NCC(=S)Nc2sc3c(c21)CCOCC3]

### a) (2-amino-4,5,7,8-tetrahydrothieno[2,3-d]oxepin-3-yl)(2,6-difluorophenyl)-methanone

To a solution of 3-(2,6-difluorophenyl)-3-oxopropanenitrile (6.18 g, 34.1 mmol) and oxepan-4-one (3.89 g, 34.1 mmol) in ethanol (150 mL) was added sulfur (1.09 g, 34.1 mmol) and morpholine (2.97 g, 2.97 mL, 34.1 mmol). The reaction mixture was stirred for 17 h at 65° C. The dark brown reaction solution was cooled and concentrated in vacuo. The residue was purified by flash column chromatography (silica, 10-100% ethyl acetate in heptane) to afford the title compound (795 mg, 7%) as a yellow solid and its regioisomer (2-amino-4,5,6,8-tetrahydrothieno[2,3-c]oxepin-3-yl)(2,6-difluorophenyl)methanone (781 mg, 7%) as a yellow solid.

Title compound: MS m/z: 310.1 [M+H]⁺, ESI pos. ¹H NMR (CDCl₃, 300 MHz) δ ppm: 2.22-2.33 (m, 2H), 2.69-2.82 (m, 2H), 3.49-3.61 (m, 2H), 3.71-3.83 (m, 2H), 6.88-7.01 (m, 2H), 7.09-7.23 (m, 2H), 7.30-7.45 (m, 1H).

Regioisomer: MS m/z: 310.1 [M+H]⁺, ESI pos. H NMR (CDCl₃, 300 MHz) δ ppm: 1.60-1.72 (m, 2H), 2.20-2.31 (m, 2H), 3.85-3.95 (m, 2H), 4.47 (s, 2H), 6.89-7.03 (m, 2H), 7.10-7.25 (m, 2H), 7.30-7.45 (m, 1H).

### b) tert-butyl (2-((3-(2,6-difluorobenzoyl)-4,5,7,8-tetrahydrothieno[2,3-d]oxepin-2-yl)amino)-2-oxoethyl)carbamate

To a solution of (2-amino-4,5,7,8-tetrahydrothieno[2,3-d]oxepin-3-yl)(2,6-difluorophenyl)-methanone (385 mg, 1.24 mmol) in dichloromethane (6 mL) was added Boc-glycine (240 mg, 1.37 mmol) and pyridine (492 mg, 0.503 mL, 6.22 mmol). The resulting yellow solution was cooled in an ice/EtOH bath (−5° C.) and phosphoroxychloride (248 mg, 0.151 mL, 1.62 mmol) was slowly added. The reaction was stirred for 1.5 h at −5° C. to 0° C. The ice-cold yellow reaction mixture was poured to a stirred bi-layer mixture of ethyl acetate (50 mL) and saturated aqueous sodium hydrogen carbonate (50 mL) and stirred vigorously for additional 15 min. The aqueous layer was extracted with ethyl acetate (50 mL). The combined organic layers were washed with brine (50 mL), dried (Na₂SO₄) and concentrated in vacuo. The residue was purified by flash column chromatography (silica, 5-100% ethyl acetate in heptane) to afford the title compound (500 mg, 80%) as a yellow solid. ¹H NMR (CDCl₃, 300 MHz) δ ppm: 12.40 (s, 1H), 7.43 (tt, 1H, J=6.3, 8.5 Hz), 6.9-7.0 (m, 2H), 5.21 (br s, 1H), 4.08 (br d, 2H, J=4.8 Hz), 3.79 (dd, 2H, J=4.3, 5.5 Hz), 3.55 (dd, 2H, J=4.1, 5.5 Hz), 2.9-2.9 (m, 2H), 2.3-2.4 (m, 2H), 1.47 (s, 9H).

### c) 2-amino-N-(3-(2,6-difluorobenzoyl)-4,5,7,8-tetrahydrothieno[2,3-d]oxepin-2-yl)-acetamide

To a solution of tert-butyl (2-((3-(2,6-difluorobenzoyl)-4,5,7,8-tetrahydrothieno[2,3-d]oxepin-2-yl)amino)-2-oxoethyl)carbamate (500 mg, 1.07 mmol) in dichloromethane (25 mL) was added trifuoroacetic acid (2.44 g, 1.65 mL, 21.4 mmol). The yellow reaction mixture was stirred for 1 h at room temperature, then concentrated in vacuo. The residue was dissolved in dichloromethane (30 mL) and aqueous sodium hydrogen carbonate (5%, 50 mL) was added. The mixture was stirred for 15 min, then the phases were separated. The aqueous layer was extracted with dichloromethane (2×30 mL). The combined organic layers were dried (Na₂SO₄) and concentrated in vacuo to afford the title compound (385 mg, 93%) as a yellow solid. MS m/z: 367.1 [M+H]⁺, ESI pos.

### d) 3-(2,6-difluorophenyl)-13-oxa-9-thia-4,7-diazatricyclo[8.5.0.02,8]pentadeca-1(10),2(8),3-triene-6-one

A mixture of 2-amino-N-(3-(2,6-difluorobenzoyl)-4,5,7,8-tetrahydrothieno[2,3-d]oxepin-2-yl)acetamide (385 mg, 1.05 mmol) in toluene (25 mL) was heated to 90° C. After 5 min, silica gel (60-200 um, 1.76 g, 1.05 mmol) was added and the mixture was stirred for 16 h at 90° C. The hot reaction suspension (60-70° C.) was filtered on a sintered funnel and the cake washed with ethyl acetate (4×50 mL). The filtrate was concentrated in vacuo to afford the title compound (360 mg, 93%) as a light yellow solid, which was used directly in the next step. MS m/z: 349.1 [M+H]⁺, ESI pos.

### e) 3-(2,6-difluorophenyl)-13-oxa-9-thia-4,7-diazatricyclo[8.5.0.02,8]pentadeca-1(10),2(8),3-triene-6-thione

To a solution of 3-(2,6-difluorophenyl)-13-oxa-9-thia-4,7-diazatricyclo[8.5.0.02,8]pentadeca-1(10),2(8),3-triene-6-one (360 mg, 1.03 mmol) in tetrahydrofuran (5 mL) was added Lawesson's reagent (251 mg, 620 μmol). The vial was sealed and the resulting yellow fine suspension was treated in a Biotage® Initiator microwave reactor for 25 min at 100° C. The resulting orange reaction solution was absorbed on Isolute HM-N (˜6 g) and purified by flash column chromatography (Isolute Flash NH2-Silica, 0-100% methanol in ethyl acetate). The resulting brownish solid was washed with diethyl ether (2×3 mL) and with heptane (4 mL) to afford the title compound (272 mg, 68%) as a light brown solid. MS m/z: 365.1 [M+H]⁺, ESI pos.

### Building Block G and Building Block H

### (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-one (Building block G)

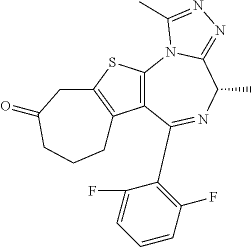 [SMILES: Cc1nnc2n1-c1sc3c(c1C(c1c(F)cccc1F)=N[C@H]2C)CCCC(=O)C3]

### (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-one (Building block H)

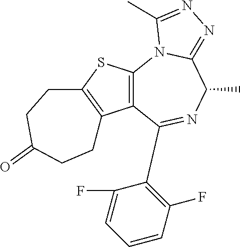 [SMILES: Cc1nnc2n1-c1sc3c(c1C(c1c(F)cccc1F)=N[C@H]2C)CCC(=O)CC3]

### a) Mixture of (2-amino-4,5,7,8-tetrahydrospiro[cyclohepta[b]thiophene-6,2′-[1,3]dioxolan]-3-yl)(2,6-difluorophenyl)methanone and (2-amino-4,5,6,8-tetrahydrospiro[cyclohepta[b]thiophene-7,2′-[1,3]dioxolan]-3-yl)(2,6-difluorophenyl)methanone

To a solution of 3-(2,6-difluorophenyl)-3-oxopropanenitrile (1.10 g, 6.07 mmol) in anhydrous ethanol (60 mL) was added 1,4-dioxaspiro[4.6]undecan-8-one (1.09 g, 6.07 mmol), sulfur (195 mg, 6.07 mmol) and morpholine (529 mg, 0.529 mL, 6.07 mmol). The mixture was stirred at 80° C. for 16 h. After cooling to room temperature, the dark brown reaction solution was concentrated in vacuo. The residue was purified by flash column chromatography (silica, 0-30% ethyl acetate in heptane) to afford the title compound (1.82 g, 82%) as a yellow solid. MS m/z: 366.1 [M+H]⁺, ESI pos.

### b) Mixture of benzyl N-[(1S)-2-[[3′-(2,6-difluorobenzoyl)spiro[1,3-dioxolane-2,6′-4,5,7,8-tetrahydrocyclohepta[b]thiophene]-2′-yl]amino]-1-methyl-2-oxo-ethyl]carbamate and benzyl N-[(1S)-2-[[3′-(2,6-difluorobenzoyl)spiro[1,3-dioxolane-2,7′-4,5,6,8-tetrahydrocyclohepta[b]thiophene]-2′-yl]amino]-1-methyl-2-oxo-ethyl]carbamate

To a solution of a regioisomeric mixture of [(2-amino-4,5,7,8-tetrahydrospiro[cyclohepta[b]thiophene-6,2′-[1,3]dioxolan]-3-yl)(2,6-difluorophenyl)methanone and (2-amino-4,5,6,8-tetrahydrospiro[cyclohepta[b]thiophene-7,2′-[1,3]dioxolan]-3-yl)(2,6-difluorophenyl)methanone] (680 mg, 1.86 mmol) in anhydrous dichloromethane (5 mL) was added ((benzyloxy)carbonyl)-L-alanine (623 mg, 2.79 mmol) and pyridine (736 mg, 0.753 mL, 9.31 mmol). The solution was cooled to 0-4° C. and phosphorus oxychloride (371 mg, 0.225 mL, 2.42 mmol) was added dropwise. The reaction mixture was stirred for 2 h at 23° C., then was extracted with dichloromethane and semi-saturated sodium hydrogen carbonate. The organic layers were washed with water and brine, dried (Na₂SO₄), filtered and concentrated in vacuo. The residue was purified by flash column chromatography (silica, 0-50% ethyl acetate in heptane) to afford the title compound (795 mg, 75%) as a light yellow foam. MS m/z: 571.3 [M+H]⁺, ESI pos.

### c) Mixture of (2S)-2-amino-N-[3′-(2,6-difluorobenzoyl)spiro[1,3-dioxolane-2,6′-4,5,7,8-tetrahydrocyclohepta[b]thiophene]-2′-yl]propanamide and (2S)-2-amino-N-[3′-(2,6-difluorobenzoyl)spiro[1,3-dioxolane-2,7′-4,5,6,8-tetrahydrocyclohepta[b]thiophene]-2′-yl]propanamide

To a solution of a regioisomeric mixture of benzyl N-[(1S)-2-[[3′-(2,6-difluorobenzoyl)spiro[1,3-dioxolane-2,6′-4,5,7,8-tetrahydrocyclohepta[b]thiophene]-2′-yl]amino]-1-methyl-2-oxo-ethyl]carbamate and benzyl N-[(1S)-2-[[3′-(2,6-difluorobenzoyl)spiro[1,3-dioxolane-2,7′-4,5,6,8-tetrahydrocyclohepta[b]thiophene]-2′-yl]amino]-1-methyl-2-oxo-ethyl]carbamate (937 mg, 1.64 mmol) in ethanol (10 mL) was added Pd/C 10% (175 mg, 0.164 mmol) under argon. The flask was evacuated and backfilled with hydrogen (balloon). The reaction mixture was stirred for 4 h at 23° C. The black suspension was filtered off, washed with ethanol, and the filtrate was concentrated in vacuo to afford the title compound (673 mg, 94%) as a yellow solid, which was used in the following step without further purification. MS m/z: 437.3 [M+H]⁺, ESI pos.

### d) Mixture of (5'S)-3′-(2,6-difluorophenyl)-5′-methyl-spiro[1,3-dioxolane-2,12′-9-thia-4,7-diazatricyclo[8.5.0.02,8]pentadeca-1(10),2(8),3-triene]-6′-one and (5'S)-3′-(2,6-difluorophenyl)-5′-methyl-spiro[1,3-dioxolane-2,13′-9-thia-4,7-diazatricyclo[8.5.0.02,8]pentadeca-1(10),2(8),3-triene]-6′-one

A regioisomeric mixture of (2S)-2-amino-N-[3′-(2,6-difluorobenzoyl)spiro[1,3-dioxolane-2,6′-4,5,7,8-tetrahydrocyclohepta[b]thiophene]-2′-yl]propanamide and (2S)-2-amino-N-[3′-(2,6-difluorobenzoyl)spiro[1,3-dioxolane-2,7′-4,5,6,8-tetrahydrocyclohepta[b]thiophene]-2′-yl]propanamide (673 mg, 1.54 mmol) in toluene (10 mL) was heated at 60° C. Silica gel (60-200 μm, 6 g) was added and the resulting yellow suspension was stirred for 20 h at 90° C. After cooling to 40° C., the mixture was filtered directly through a sintered funnel and the filter cake (silica) was rinsed with ethyl acetate. The filtrate was concentrated in vacuo to afford the title compound (660 mg, quant.) as a yellow solid, which was used in the following step without further purification. MS m/z: 419.3 [M+H]⁺, ESI pos.

### e) Mixture of (7'S)-9′-(2,6-difluorophenyl)-3′,7′-dimethyl-spiro[1,3-dioxolane-2,15′-18-thia-2,4,5,8-tetrazatetracyclo[8.8.0.02,6.011,17]octadeca-1(10),3,5,8,11(17)-pentaene] and (7'S)-9′-(2,6-difluorophenyl)-3′7′-dimethyl-spiro[1,3-dioxolane-2,14′-18-thia-2,4,5,8-tetrazatetracyclo[8.8.0.02,6.011,17]octadeca-1(10),3.5.8.11(17)-pentaene]

A regioisomeric mixture of (5'S)-3′-(2,6-difluorophenyl)-5′-methyl-spiro[1,3-dioxolane-2,12′-9-thia-4,7-diazatricyclo[8.5.0.02,8]pentadeca-1(10),2(8),3-triene]-6′-one and (5'S)-3′-(2,6-difluorophenyl)-5′-methyl-spiro[1,3-dioxolane-2,13′-9-thia-4,7-diazatricyclo[8.5.0.02,8]pentadeca-1(10),2(8),3-triene]-6′-one (1.055 g, 2.52 mmol) was dissolved in tetrahydrofuran (50 mL). The solution was cooled to 0-4° C., before addition of sodium hydride (60% dispersion in mineral oil, 202 mg, 5.04 mmol). The resulting light yellow suspension was stirred for 10 min at 0-4° C., then bis(2-oxo-3-oxazolidinyl)phosphinic chloride (1.28 g, 5.04 mmol) was added and the reaction mixture was stirred for 2 h at 0-4° C. to form a dark yellow suspension. Acethydrazide (519 mg, 6.30 mmol) was added then the reaction mixture was stirred at 23° C. for 1 h. Another portion of acethydrazide (519 mg, 6.30 mmol) was added and stirring continued for 2 h at 23° C., resulting in a dark orange suspension. The reaction mixture was stirred for 16 h at 60° C., before being cooled to room temperature and quenched by addition of aqueous ammonium chloride. The mixture was extracted twice with ethyl acetate and the combined organic layers were washed with water and brine, dried (Na₂SO₄), filtered and concentrated in vacuo. The residue was purified by flash column chromatography (silica, 0-50% ethyl acetate in heptane, then ethyl acetate/methanol 9:1) to afford the title compound (655 mg, 95%) as a light brown foam. MS m/z: 457.2 [M+H]⁺, ESI pos.

### f) (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-one (Building block

### G) and (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-one (Building block

A regioisomeric mixture of (7'S)-9′-(2,6-difluorophenyl)-3′,7′-dimethyl-spiro[1,3-dioxolane-2,15′-18-thia-2,4,5,8-tetrazatetracyclo[8.8.0.02,6.011,17]octadeca-1(10),3,5,8,11(17)-pentaene] and (7'S)-9′-(2,6-difluorophenyl)-3′,7′-dimethyl-spiro[1,3-dioxolane-2,14′-18-thia-2,4,5,8-tetrazatetracyclo[8.8.0.02,6.011,17]octadeca-1(10),3,5,8,11(17)-pentaene] (440 mg, 0.964 mmol) was dissolved in acetone (5 mL), then acetic acid (4 mL) and water (5 mL) were added. The mixture was stirred under microwave irradiation for 60 min at 120° C. After cooling, the reaction mixture was poured into ice water and basified by addition of solid sodium hydrogen carbonate. The aqueous layer was extracted twice with ethyl acetate. The combined organic layers were washed with water and brine, dried (Na₂SO₄), filtered and concentrated in vacuo. The residue was purified and regioisomers separated by preparative HPLC (Gemini NX, acetonitrile/water (with 0.1% formic acid)) to afford (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-15-one (Building block G, 153 mg, 39%) as a light yellow foam and (7S)-9-(2,6-difluorophenyl)-3,7-dimethyl-18-thia-2,4,5,8-tetrazatetracyclo [8.8.0.02,6.011,17]octadeca-1(10),3,5,8,11(17)-pentaen-14-one (Building Block H, 106 mg, 27%) as a light yellow foam.

### Building Block G

MS m/z: 413.1 [M+H]⁺, ESI pos.

¹H NMR (DMSO-d₆, 600 MHz) δ ppm: ppm 7.51-7.59 (m, 1H), 7.23 (br s, 2H), 4.40 (d, J=6.6 Hz, 1H), 3.96-4.01 (m, 1H), 3.88-3.94 (m, 1H), 2.52-2.60 (m, 5H), 2.36-2.44 (m, 1H), 2.00-2.08 (m, 1H), 1.84-1.91 (m, 1H), 1.86 (d, J=6.6 Hz, 3H), 1.61-1.71 (m, 1H).

### Building Block H

MS m/z: 413.1 [M+H]⁺, ESI pos.

¹H NMR (DMSO-d₆, 600 MHz) δ ppm: 7.55-7.57 (m, 1H), 6.79-7.48 (m, 2H), 4.40 (q, J=6.6 Hz, 1H), 3.09-3.18 (m, 2H), 2.68-2.75 (m, 1H), 2.57-2.64 (m, 1H), 2.57-2.66 (m, 4H), 2.52-2.56 (m, 1H), 2.33 (ddd, J=13.9, 8.8, 3.3 Hz, 1H), 2.15 (ddd, J=16.1, 9.6, 3.2 Hz, 1H), 1.87 (d, J=6.6 Hz, 3H).

### Building Block I

### 3-(2,6-difluorophenyl)-11-oxa-9-thia-4,7-diazatricyclo[8.5.0.02,8]pentadeca-1(10),2(8),3-trien-6-thione

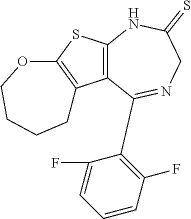 [SMILES: Fc1cccc(F)c1C1=NCC(=S)Nc2sc3c(c21)CCCCO3]

### a) (7-amino-2,3,4,5-tetrahydrothieno[2,3-b]oxepin-6-yl)(2,6-difluorophenyl)methanone

To a solution of 3-(2,6-difluorophenyl)-3-oxopropanenitrile (524 mg, 2.89 mmol) in anhydrous ethanol (15 mL) was added oxepan-3-one (CAS 130722-42-8; 330 mg, 2.89 mmol), sulfur (92.7 mg, 2.89 mmol) and morpholine (252 mg, 0.253 mL, 2.89 mmol). The mixture was stirred at 80° C. for 16 h and then concentrated in vacuo. The residue was purified by flash column chromatography (silica, 0-40% ethyl acetate in heptane) to afford the title compound (547 mg, 61%) as a yellow solid. MS m/z: 310.1 [M+H]⁺, ESI pos.

### b) tert-butyl (2-((6-(2,6-difluorobenzoyl)-2,3,4,5-tetrahydrothieno[2,3-b]oxepin-7-yl)amino)-2-oxoethyl)carbamate

To a solution of (7-amino-2,3,4,5-tetrahydrothieno[2,3-b]oxepin-6-yl)(2,6-difluorophenyl)-methanone (210 mg, 0.679 mmol) in anhydrous dichloromethane (4 mL), (tert-butoxycarbonyl)-glycine (131 mg, 747 μmol) was added. The reaction mixture was cooled to 0° C., before addition of pyridine (269 mg, 0.275 mL, 3.39 mmol). After 5 min, phosphoryl chloride (135 mg, 0.0823 mL, 883 mmol) was added and the reaction mixture was stirred at 0° C. for 1 h. The reaction mixture was poured into a stirred mixture of ethyl acetate, ice-water and saturated aqueous sodium hydrogencarbonate. After 5 min, the aqueous phase was extracted with ethyl acetate twice. The combined organic layers were washed with brine, dried (Na₂SO₄), filtered and concentrated in vacuo. The residue was purified by flash column chromatography (Si—NH2 column, 0-40% ethyl acetate in heptane) to afford the title compound (202 mg, 64%) as a light yellow solid. MS m/z: 465.3 [M−H]⁺, ESI neg.

### c) 2-amino-N-(6-(2,6-difluorobenzoyl)-2,3,4,5-tetrahydrothieno[2,3-b]oxepin-7-yl)acetamide

To a mixture of tert-butyl (2-((6-(2,6-difluorobenzoyl)-2,3,4,5-tetrahydrothieno[2,3-b]oxepin-7-yl)amino)-2-oxoethyl)carbamate (197 mg, 0.422 mmol) in dichloromethane (5 mL) was added trifluoroacetic acid (963 mg, 0.647 mL, 8.45 mmol) and the reaction mixture was stirred at room temperature for 2.5 h. The mixture was concentrated in vacuo and the residue was dissolved in dichloromethane (30 mL). Aqueous sodium hydrogen carbonate (5%, 20 mL) was added and the mixture was stirred for 10 min. The aqueous layer was extracted with dichloromethane (2×30 mL). The combined organic layers were dried (Na₂SO₄), filtered and concentrated in vacuo to afford the title compound (155 mg, 100%) as a yellow solid. MS m/z: 367.1 [M+H]⁺, ESI pos.

### d) 3-(2,6-difluorophenyl)-11-oxa-9-thia-4,7-diazatricyclo[8.5.0.02,8]pentadeca-1(10),2(8),3-trien-6-one

To a solution of 2-amino-N-(6-(2,6-difluorobenzoyl)-2,3,4,5-tetrahydrothieno[2,3-b]oxepin-7-yl)acetamide (155 mg, 0.413 mmol) in toluene (7 mL) was added silica gel (0.063-0.2 mm, 750 mg, 0.413 mmol). The mixture was stirred at 90° C. overnight, before being cooled to 40° C. The warm mixture was filtered directly through a sintered funnel and the filter cake (silica) was rinsed with ethyl acetate. The filtrate was concentrated in vacuo to afford the title compound (125 mg, 87%) as a light brown solid, which was used in the next step without further purification. MS m/z: 349.1 [M+H]⁺, ESI pos.

### e) 3-(2,6-difluorophenyl)-11-oxa-9-thia-4,7-diazatricyclo[8.5.0.02,8]pentadeca-1(10),2(8),3-trien-6-thione

To a mixture of 3-(2,6-difluorophenyl)-11-oxa-9-thia-4,7-diazatricyclo[8.5.0.02,8] pentadeca-1(10),2(8),3-trien-6-one (120 mg, 0.344 mmol) in acetonitrile (3 mL) was added Lawesson's reagent (83.6 mg, 0.207 mmol). The reaction mixture was stirred in the microwave at 100° C. for 20 min, before being concentrated in vacuo. The residue was purified by flash column chromatography (silica, 0-40% ethyl acetate in heptane) to afford the title compound (125 mg, 100%) as a yellow solid. MS m/z: 365.1 [M+H]⁺, ESI pos.

### Building Block J

### (5S)-3-(2,6-difluorophenyl)-5-methyl-11-oxa-9-thia-4,7-diazatricyclo[8.5.0.02,8]pentadeca-1(10),2(8),3-triene-6-thione

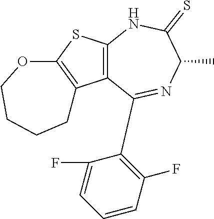 [SMILES: C[C@@H]1N=C(c2c(F)cccc2F)c2c(sc3c2CCCCO3)NC1=S]

### a) tert-butyl N-[(1S)-2-[[6-(2,6-difluorobenzoyl)-2,3,4,5-tetrahydrothieno[2,3-b]oxepin-7-yl]amino]-1-methyl-2-oxo-ethyl]carbamate

To a solution of (tert-butoxycarbonyl)-L-alanine (196 mg, 1.03 mmol) in N,N-dimethylformamide (2 mL) was added at room temperature DIPEA (401 mg, 0.542 mL, 3.1 mmol) followed by HATU (511 mg, 1.34 mmol). After stirring for 5 min, (7-amino-2,3,4,5-tetrahydrothieno[2,3-b]oxepin-6-yl)(2,6-difluorophenyl)methanone (320 mg, 1.03 mmol) was added and the reaction mixture was stirred at 50° C. for 16 h. The reaction mixture was concentrated in vacuo and the residue was partitioned between ethyl acetate and water. The aqueous phase was extracted with ethyl acetate and the combined organic layers were purified by flash column chromatography (silica, 0-30% ethyl acetate in heptane) to afford the title compound (214 mg, 43%) as a yellow solid. MS m/z: 489.2 [M−H]⁺, ESI neg.

### b) (2S)-2-amino-N-[6-(2,6-difluorobenzoyl)-2,3,4,5-tetrahydrothieno[2,3-b]oxepin-7-yl]propanamide

To a solution of tert-butyl N-[(1S)-2-[[6-(2,6-difluorobenzoyl)-2,3,4,5-tetrahydrothieno[2,3-b]oxepin-7-yl]amino]-1-methyl-2-oxo-ethyl]carbamate (214 mg, 0.445 mmol) in dichloromethane (6.5 mL) was added dropwise trifluoroacetic acid (1.02 g, 0.662 mL, 8.91 mmol). The reaction mixture was stirred at room temperature overnight, then concentrated in vacuo. The residue was diluted with dichloromethane and extracted with saturated aqueous sodium hydrogen carbonate. The aqueous phase was washed twice with dichloromethane. The combined organic layers were dried (Na₂SO₄), filtered and concentrated in vacuo to afford the title compound (169 mg, 100%) as a yellow solid. MS m/z: 381.1 [M+H]⁺, ESI pos.

### c) (5S)-3-(2,6-difluorophenyl)-5-methyl-11-oxa-9-thia-4,7-diazatricyclo[8.5.0.02,8]pentadeca-1(10),2(8),3-triene-6-one

To a solution of (2S)-2-amino-N-[6-(2,6-difluorobenzoyl)-2,3,4,5-tetrahydrothieno[2,3-b]oxepin-7-yl]propanamide (169 mg, 0.402 mmol) in toluene (3 mL) at 60° C. was added silica gel (0.063-0.2 mm, 875 mg, 0.402 mmol) and the reaction mixture was stirred at 90° C. overnight, before being cooled to 40° C. The mixture was filtered directly through a sintered funnel and the filter cake (silica) was rinsed with ethyl acetate. The filtrate was concentrated in vacuo to afford the title compound (150 mg, 88%, 85% pure) as a yellow solid. MS m/z: 363.3 [M+H]⁺, ESI pos

### d) (5S)-3-(2,6-difluorophenyl)-5-methyl-11-oxa-9-thia-4,7-diazatricyclo[8.5.0.02,8] pentadeca-1(10),2(8),3-triene-6-thione

To a mixture of (5S)-3-(2,6-difluorophenyl)-5-methyl-11-oxa-9-thia-4,7-diazatricyclo[8.5.0.02,8]pentadeca-1(10),2(8),3-triene-6-one (150 mg, 0.352 mmol) in acetonitrile (3 mL) was added Lawesson's reagent (85.4 mg, 0.211 mmol). The reaction mixture was stirred in the microwave at 100° C. for 30 min, then concentrated in vacuo. The residue was purified by flash column chromatography (silica, 0-40% ethyl acetate in heptane) to afford the title compound (106 mg, 80%) as a yellow solid. MS m/z: 379.1 [M+H]⁺, ESI pos

### Building Block K

### (13S)-15-(2,6-difluorophenyl)-13-methyl-4,7-dioxa-9-thia-11,14-diazatricyclo[8.5.0.02,8]pentadeca-1(10),2(8),14-triene-12-one

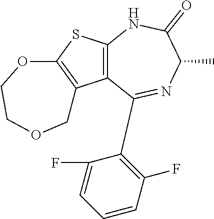 [SMILES: C[C@@H]1N=C(c2c(F)cccc2F)c2c(sc3c2COCCO3)NC1=O]

### a) (7-amino-2,3-dihydro-5H-thieno[2,3-e][1,4]dioxepin-6-yl)(2,6-difluorophenyl)methanone

To a solution of 3-(2,6-difluorophenyl)-3-oxopropanenitrile (1.87 g, 10.3 mmol) in anhydrous ethanol (20 mL) was added 1,4-dioxepan-6-one (CAS-No. 28544-93-6; 2 g, 10.3 mmol), sulfur (331 mg, 10.3 mmol) and morpholine (900 mg, 0.904 mL, 10.3 mmol). The mixture was stirred in a sealed tube at 80° C. for 16 h, then concentrated in vacuo. The residue was purified by flash column chromatography (silica, 0-30% ethyl acetate in heptane) to afford the title compound (1.66 g, 52%) as a yellow solid. MS m/z: 312.1 [M+H]⁺, ESI pos.

### b) tert-butyl N-[(1S)-2-[[6-(2,6-difluorobenzoyl)-3,5-dihydro-2H-thieno[2,3-e][1,4]dioxepin-7-yl]amino]-1-methyl-2-oxo-ethyl]carbamate

To a solution of (tert-butoxycarbonyl)-L-alanine (608 mg, 3.21 mmol) and N,N-dimethylformamide (10 mL) was added at room temperature Hunig's base (1.25 g, 1.68 mL, 9.64 mmol) and HATU (1.59 g, 4.18 mmol). The resulting light yellow solution was stirred for 5 min, before addition of (7-amino-2,3-dihydro-5H-thieno[2,3-e][1,4]dioxepin-6-yl)(2,6-difluorophenyl)-methanone (1 g, 3.21 mmol). The reaction mixture was stirred at room temperature overnight, then concentrated in vacuo. The residue was partitioned between ethyl acetate and water. The aqueous phase was washed with ethyl acetate and the combined organic layers were washed with brine, dried (Na₂SO₄) and concentrated in vacuo. The residue was purified by flash column chromatography (silica, 0-40% ethyl acetate in heptane) to afford the title compound (1.39 g, 90%) as a yellow solid. MS m/z: 481.2 [M−H]⁺, ESI neg.

### c) (2S)-2-amino-N-[6-(2,6-difluorobenzoyl)-3,5-dihydro-2H-thieno[2,3-e][1,4]dioxepin-7-yl]propanamide

To a mixture of tert-butyl N-[(1S)-2-[[6-(2,6-difluorobenzoyl)-3,5-dihydro-2H-thieno[2,3-e][1,4]dioxepin-7-yl]amino]-1-methyl-2-oxo-ethyl]carbamate (1.39 g, 2.88 mmol) in dichloromethane (25 mL) was added dropwise trifluoroacetic acid (6.57 g, 4.3 mL, 57.6 mmol). The reaction mixture was stirred at room temperature overnight, then concentrated in vacuo. The residue was partitioned between dichloromethane and saturated aqueous sodium hydrogen carbonate. The aqueous phase was extracted twice with dichloromethane. The combined organic layers were dried (Na₂SO₄) and concentrated in vacuo to afford the title compound (830 mg, 75%) as a yellow solid. MS m/z: 383.0 [M+H]⁺, ESI pos.

### d) (13S)-15-(2,6-difluorophenyl)-13-methyl-4,7-dioxa-9-thia-11,14-diazatricyclo[8.5.0.02,8]pentadeca-1(10),2(8),14-triene-12-one

To a solution of (2S)-2-amino-N-[6-(2,6-difluorobenzoyl)-3,5-dihydro-2H-thieno[2,3-e][1,4]dioxepin-7-yl]propanamide (827 mg, 2.16 mmol) in toluene (14.5 mL) at 60° C. was added silica gel (0.063-0.2 mm, 4.24 g). The orange mixture was stirred at 90° C. overnight, before being cooled to 40° C. The mixture was filtered directly through a sintered funnel and the filter cake (silica) was rinsed with ethyl acetate. The filtrate was concentrated in vacuo to afford the crude title compound (776 mg, 88%, 90% pure) as a light brown solid. MS m/z: 365.2 [M+H]⁺, ESI pos.

### Building Block L

### (13S)-15-(2,6-difluorophenyl)-13-methyl-4,7-dioxa-9-thia-11,14-diazatricyclo[8.5.0.02,8]pentadeca-1(10),2(8),14-triene-12-thione

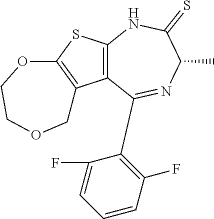 [SMILES: C[C@@H]1N=C(c2c(F)cccc2F)c2c(sc3c2COCCO3)NC1=S]

To a solution of (13$)-15-(2,6-difluorophenyl)-13-methyl-4,7-dioxa-9-thia-11,14-diazatricyclo[8.5.0.02,8]pentadeca-1(10),2(8),14-triene-12-one (Building block K, 776 mg, 2.13 mmol) in acetonitrile (12 mL) was added Lawesson's reagent (517 mg, 1.28 mmol). The reaction mixture was stirred under microwave irradiation at 100° C. for 30 min, then concentrated in vacuo. The residue was purified by flash column chromatography (silica, 6% methanol in dichloromethane) to afford the title compound (593 mg, 73%) as a light brown solid. MS m/z: 381.1 [M+H]⁺, ESI pos.

### Building Block M

### (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one

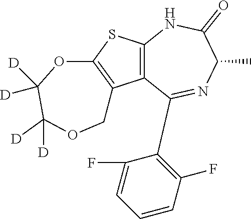 [SMILES: [2H]C1([2H])OCc2c(sc3c2C(c2c(F)cccc2F)=N[C@@H](C)C(=O)N3)OC1([2H])[2H]]

### a) ethyl 2-[1,1,2,2-tetradeuterio-2-(2-ethoxy-2-oxo-ethoxy)ethoxy]acetate

To a mixture of boron trifluoride diethyl ether complex (0.54 g, 2.27 mmol) and dichloromethane (500 mL) was added 1,1,2,2-tetradeuterioethane-1,2-diol (10.0 g, 151.3 mmol) at 0° C. over 10 minutes. The reaction mixture was allowed to warm to room temperature and stirred for 20 min. The mixture was re-cooled to 0° C. and a solution of ethyl diazoacetate in toluene (34.7 g, 304.1 mmol) was added dropwise over 15 minutes. The reaction mixture was stirred at 25° C. for 5 h, before being quenched by addition of water (50 mL). The mixture was diluted with water (300 mL) and ethyl acetate (1000 mL), then the layers were separated. The aqueous layer was further extracted with ethyl acetate (2×200 mL). The combined organic layers were washed with brine (60 mL), dried (Na₂SO₄) and concentrated in vacuo. The residue was purified by flash column chromatography (silica, 0-50% ethyl acetate in petroleum ether) to afford the title compound (28 g, 77%) as a light yellow oil. MS m/z: 239.1 [M+H]⁺, ESI pos

### b) ethyl 2,2,3,3-tetradeuterio-6-oxo-1,4-dioxepane-5-carboxylate

To a solution of ethyl 2-[1,1,2,2-tetradeuterio-2-(2-ethoxy-2-oxo-ethoxy)ethoxy]acetate (72.0 g, 302.18 mmol) in N,N-dimethylformamide (1300 mL) was added lithium tert-butoxide (48.38 g, 604.36 mmol) and the mixture was stirred at 85° C. for 5 h. The mixture was diluted with ethyl acetate (3000 mL). The organic layer was washed with water (2000 mL), and the aqueous layer was extracted with ethyl acetate (3×1000 mL). The combined organic layers were dried (Na₂SO₄) and concentrated in vacuo to afford the title compound (50 g, 51%) as a light brown oil. MS m/z: 193.2 [M+H]⁺, ESI pos.

### c) 2,2,3,3-tetradeuterio-1,4-dioxepan-6-one

A mixture of ethyl 2,2,3,3-tetradeuterio-6-oxo-1,4-dioxepane-5-carboxylate (35.54 g, 184.93 mmol) in aqueous hydrochloric acid (10%, 500 mL) was stirred at 100° C. for 3 h. The mixture was treated with saturated aqueous sodium carbonate to pH=7 and extracted with ethyl acetate (3×300 mL), washed with brine (50 mL), dried (Na₂SO₄) and concentrated in vacuo to afford the title compound (20 g, 81%, 90% purity) as a dark brown liquid. MS m/z: 570.7 [M+H]⁺, ESI pos.

### d) (7-amino-2,2,3,3-tetradeuterio-5H-thieno[2,3-e][1,4]dioxepin-6-yl)-(2,6-difluorophenyl)methanone

A mixture of 2,2,3,3-tetradeuterio-1,4-dioxepan-6-one (8.0 g, 59.93 mmol), 3-(2,6-difluorophenyl)-3-oxo-propanenitrile (10.86 g, 59.93 mmol), morpholine (5.22 g, 59.93 mmol) and sulfur (1.92 g, 59.93 mmol) in ethanol (100 mL) was heated to 75° C. in a sealed tube for 14 h. After cooling to room temperature, the reaction mixture was poured into water and extracted with ethyl acetate (3×50 mL). The organic layers were dried (Na₂SO₄) and concentrated in vacuo. The residue was purified by flash column chromatography (silica, 0-50% ethyl acetate in petroleum ether) to afford the title compound (6.8 g, 36%) as a yellow solid. MS m/z: 316.0 [M+H]⁺, ESI pos.

### e) tert-butyl N-[(1S)-1-methyl-2-oxo-2-[[2,2,3,3-tetradeuterio-6-(2,6-difluorobenzoyl)-5H-thieno[2,3-e][1,4]dioxepin-7-yl]amino]ethyl]carbamate

To a solution of Boc-L-Ala-OH (8.16 g, 43.13 mmol) in N,N-dimethylformamide (50 mL) was added at room temperature (7-amino-2,2,3,3-tetradeuterio-5H-thieno[2,3-e][1,4]dioxepin-6-yl)-(2,6-difluorophenyl)-methanone (6.8 g, 21.56 mmol) followed by DIPEA (13.91 g, 107.82 mmol). After 1 min stirring, HBTU (17.17 g, 45.29 mmol) was added and the mixture was stirred at 25° C. for 14 h, then concentrated in vacuo. The residue was partitioned between ethyl acetate (20 mL) and water and brine. The organic layer was separated, dried (Na₂SO₄) and concentrated in vacuo to afford the title compound (10 g, 76%, 80% purity) as a light brown oil. The crude product was used in the next step without further purification, MS m/z: 509 [M+H]⁺, ESI pos.

### f) (2S)-2-amino-N-[2,2,3,3-tetradeuterio-6-(2,6-difluorobenzoyl)-5H-thieno[2,3-e][1,4]dioxepin-7-yl]propanamide

To a mixture of tert-butyl N-[(1S)-1-methyl-2-oxo-2-[[2,2,3,3-tetradeuterio-6-(2,6-difluorobenzoyl)-5H-thieno[2,3-e][1,4]dioxepin-7-yl]amino]ethyl]carbamate (9.0 g, 18.5 mmol) in dichloromethane (100 mL) was added trifluoroacetic acid (28.5 mL, 370 mmol). The mixture was stirred at room temperature for 12 h, before being concentrated in vacuo. The residue was dissolved in dichloromethane (100 mL), then treated with saturated aqueous sodium hydrogen carbonate to pH=7. The aqueous layer was extracted with dichloromethane (3×50 mL). The combined organic layers were washed with brine (50 mL), dried (Na₂SO₄) and concentrated in vacuo to afford the title compound (4 g, 44%) as a yellow solid. MS m/z: 387.0 [M+H]⁺, ESI pos.

### g) (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one

To a mixture of (2S)-2-amino-N-[2,2,3,3-tetradeuterio-6-(2,6-difluorobenzoyl)-5H-thieno[2,3-e][1,4]dioxepin-7-yl]propanamide (4.0 g, 8.28 mmol) in toluene (50 mL) was added silica gel (12.42 g, 207.03 mmol) and 4 Å molecular sieves (3.0 g, 8.28 mmol). The mixture was stirred at 100° C. under nitrogen for 12 h. The mixture was filtered directly through a sintered funnel and the filter cake was rinsed with ethyl acetate. The filtrate was concentrated in vacuo to afford the title compound (900 mg, 27%) as a dark brown solid. MS m/z: 369.1 [M+H]⁺, ESI pos.

### Building Block N

(6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one

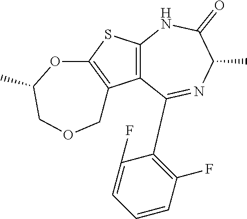

### a) ethyl 2-[(2S)-2-(2-ethoxy-2-oxo-ethoxy)propoxy]acetate

To a suspension of sodium hydride (60% dispersion in mineral oil, 27.6 g, 690.0 mmol) in anhydrous tetrahydrofuran (500 mL) was added at 0° C. over 10 min (S)-(+)-1,2-propanediol (25.0 g, 328.6 mmol). The reaction mixture was allowed to warm to room temperature and stirred for 20 min, before being re-cooled to 0° C. and ethyl bromoacetate (74.7 mL, 673.5 mmol) was added dropwise over 15 min. The reaction mixture was allowed to warm to room temperature and stirred for 14 h, before being quenched by addition of water (50 mL). The mixture was diluted with water (300 mL) and ethyl acetate (1000 mL), then the layers were separated. The aqueous layer was further extracted with ethyl acetate (2×200 mL). The combined organic layers were washed with brine (60 mL), dried (Na₂SO₄), filtered and concentrated in vacuo. The residue was purified by flash column chromatography (silica, petroleum ether/ethyl acetate, 5:1 to 3:1) to afford the title compound (22.6 g, 28%) as a colorless liquid. MS m/z: 249.0 [M+H]⁺, ESI pos.

### b) (2S)-2-methyl-6-oxo-1,4-dioxepane-5-carboxylic acid

To a solution of ethyl 2-[(2S)-2-(2-ethoxy-2-oxo-ethoxy)propoxy]acetate (26.0 g, 104.7 mmol) in anhydrous tetrahydrofuran (500 mL) was added lithium tert-butoxide (16.8 g, 209.4 mmol) and the reaction mixture was stirred at 85° C. for 5 h. The mixture was neutralized with acetic acid to pH=7, then with aqueous hydrochloric acid (10 wt. %) to pH=5. The aqueous layer was further extracted with ethyl acetate (3×200 mL). The combined organic layers were dried (Na₂SO₄), filtrated and concentrated in vacuo to afford the title compound (20 g, 47%) as light brown liquid. The crude product was used in the next step without further purification. MS m/z: 203.0 ([M+H]⁺), ESI pos.

### c) (2S)-2-methyl-1,4-dioxepan-6-one

To a mixture of (2S)-2-methyl-6-oxo-1,4-dioxepane-5-carboxylic acid (20.0 g, 98.9 mmol) was added aqueous hydrochloric acid (10 wt. %, 300 mL) and the mixture was stirred at 100° C. for 3 h. The mixture was neutralized with saturated aqueous sodium carbonate to pH=7. The aqueous layer was further extracted with ethyl acetate (3×500 mL). The combined organic layers were washed with brine (200 mL), dried (Na₂SO₄), filtered and the filtrate was concentrated in vacuo. The residue was purified by flash column chromatography (silica gel, petroleum ether/ethyl acetate, 10:1 to 3:1) to afford the title compound (6.8 g, 53%) as a colorless oil. ¹H NMR (CDCl₃, 400 MHz) δ ppm: 4.33-4.15 (m, 4H), 4.11-4.04 (m, 1H), 3.86 (dqd, J=12.8, 6.4, 2.3 Hz, 1H), 3.39 (dd, J=12.7, 9.8 Hz, 1H), 1.16 (d, J=6.4 Hz, 3H).

### d) [(2S)-7-amino-2-methyl-3,5-dihydro-2H-thieno[2,3-e][1,4]dioxepin-6-yl]-(2,6-difluorophenyl)methanone

A mixture of 3-(2,6-difluorophenyl)-3-oxo-propanenitrile (9.46 g, 52.2 mmol), (2S)-2-methyl-1,4-dioxepan-6-one (6.8 g, 52.2 mmol), morpholine (4.55 g, 52.2 mmol) and sulfur (1.67 g, 52.2 mmol) in ethanol (60 mL) was heated to 80° C. in a sealed tube for 14 h. After cooling to room temperature, the reaction mixture was poured into water. The aqueous layer was extracted with ethyl acetate (3×150 mL). The combined organic layers were washed with brine (30 mL), dried (Na₂SO₄), filtered and the filtrate was concentrated in vacuo. The residue was purified by flash column chromatography (silica, 0-50% ethyl acetate in petroleum ether), then regioisomers separated by chiral SFC (Daicel CHIRALPAK® AS-H, 25% ethanol) to afford the title compound (1.8 g, 11%) as yellow solid and its regioisomer [(3S)-7-amino-3-methyl-3,5-dihydro-2H-thieno[2,3-e][1,4]dioxepin-6-yl]-(2,6-difluorophenyl)methanone (2.0 g, 12%) as yellow solid.

Title compound: MS m/z: 326.1 ([M+H]⁺), ESI pos. ¹H NMR (CDCl₃, 400 MHz) δ ppm: 7.41-7.23 (m, 3H), 6.94 (t, J=8.1 Hz, 2H), 4.14-3.92 (m, 3H), 3.80 (ddd, J=8.3, 6.6, 1.8 Hz, 1H), 3.61 (dd, J=12.1, 8.3 Hz, 1H), 1.10 (d, J=6.5 Hz, 3H).

Regioisomer: MS m/z: 326.1 ([M+H]⁺), ESI pos. ¹H NMR (CDCl₃, 400 MHz) δ ppm: 7.36 (tt, J=8.4, 6.4 Hz, 1H), 7.25 (d, J=8.6 Hz, 2H), 6.95 (dd, J=8.3, 7.7 Hz, 2H), 4.14 (ddd, J=6.6, 5.5, 1.9 Hz, 1H), 4.07-3.84 (m, 3H), 3.51 (dd, J=13.1, 8.4 Hz, 1H), 1.22 (d, J=6.6 Hz, 3H).

### e) tert-butyl N-[(1S)-2-[[(2S)-6-(2,6-difluorobenzoyl)-2-methyl-3,5-dihydro-2H-thieno[2,3-e][1,4]dioxepin-7-yl]amino]-1-methyl-2-oxo-ethyl]carbamate

To a solution of (tert-butoxycarbonyl)-L-alanine (1.2 g, 6.1 mmol) in anhydrous dichloromethane (30 mL) was added HOBT (0.90 g, 6.6 mmol) and EDC (1.2 g, 6.6 mmol) at 0° C. The mixture was stirred at 0° C. for 10 min, before addition of [(2S)-7-amino-2-methyl-3,5-dihydro-2H-thieno[2,3-e][1,4]dioxepin-6-yl]-(2,6-difluorophenyl)methanone (1.8 g, 5.5 mmol) and DIPEA (2.1 g, 16.6 mmol). The reaction mixture was stirred at room temperature for 12 h, before being quenched by addition of water (30 mL). The aqueous layer was extracted with ethyl acetate (3×50 mL). The combined organic layers were washed with brine (50 mL), dried (Na₂SO₄), filtered and the filtrate was concentrated in vacuo. The residue was purified by flash column chromatography (silica, petroleum ether/ethyl acetate 5:1 to 1:5) to afford the title compound (1.9 g, 66%) as a yellow solid. MS m/z: 519.0 ([M+Na]⁺), ESI pos.

### f) (2S)-2-amino-N-[(2S)-6-(2,6-difluorobenzoyl)-2-methyl-3,5-dihydro-2H-thieno[2,3-e][1,4]dioxepin-7-yl]propanamide

To a solution of tert-butyl N-[(1S)-2-[[(2S)-6-(2,6-difluorobenzoyl)-2-methyl-3,5-dihydro-2H-thieno[2,3-e][1,4]dioxepin-7-yl]amino]-1-methyl-2-oxo-ethyl]carbamate (1.9 g, 3.6 mmol) in dichloromethane (10 mL) was added trifluoroacetic acid (5.6 mL). The reaction mixture was stirred at room temperature for 6 h, before being concentrated in vacuo. The residue was partitioned between dichloromethane (10 mL) and saturated aqueous sodium hydrogen carbonate (to pH=7). The aqueous layer was extracted with dichloromethane (3×30 mL). The combined organic layers were washed with brine (50 mL), dried (Na₂SO₄) and concentrated in vacuo to afford the title compound (1.3 g, 84%) as a yellow solid. MS m/z: 397.0 ([M+H]⁺), ESI pos.

### g) (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one

To a solution of (2S)-2-amino-N-[(2S)-6-(2,6-difluorobenzoyl)-2-methyl-3,5-dihydro-2H-thieno[2,3-e][1,4]dioxepin-7-yl]propanamide (1.3 g, 3.1 mmol) in toluene (30 mL) was added silica gel (100-200 mesh, 2.0 g) and 4 Å molecular sieves (1.0 g). The mixture was stirred at 100° C. under nitrogen for 12 h, before being cooled to room temperature. The mixture was filtered directly through a sintered funnel and the filter cake was rinsed with ethyl acetate. The filtrate was concentrated in vacuo to afford the title compound (1.1 g, 85%) as a yellow solid. MS m/z: 379.3 ([M+H]⁺), ESI pos.

### Building Block O

### (13S)-15-(2,6-difluorophenyl)-13-methyl-3-oxa-9-thia-11,14-diazatricyclo[8.5.0.02,8]-pentadeca-1(10),2(8),14-triene-12-thione

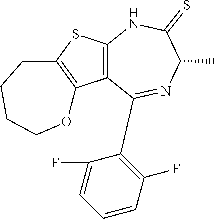 [SMILES: C[C@@H]1N=C(c2c(F)cccc2F)c2c(sc3c2OCCCC3)NC1=S]

### a) 6-chloro-2,3,4,5-tetrahydrooxepine-7-carbaldehyde

To a mixture of N,N-dimethylformamide (5.12 g, 5.42 mL, 70.1 mmol) in anhydrous dichloromethane (40 mL) was added phosphorous oxychloride (10.7 g, 6.53 mL, 70.1 mmol) dropwise at 0° C. The mixture was stirred for 30 min at 0° C., before being allowed to warm to room temperature. After 30 min, a solution of oxepan-3-one (4.0 g, 35.0 mmol) in dichloromethane (5 mL) was added dropwise and the reaction mixture was stirred at 40° C. for 3 h. The mixture was quenched by addition of saturated aqueous sodium hydrogen carbonate (250 mL) and extracted with dichloromethane (3×200 mL). The combined organic layers were washed with brine (50 mL), dried (Na₂SO₄) and concentrated in vacuo. The residue was purified by flash column chromatography (silica, heptane/ethyl acetate, 0-30%) to afford the title compound (5.30 mg, 97%) as a light brown oil. MS m/z: 161.0 ([{³⁵Cl}M+H]⁺), 163.0 ([{³¹Cl}M+H]⁺), ESI pos.

### b) 3-thiocyanato-4,5,6,7-tetrahydrooxepine-2-carbaldehyde

To a solution of 6-chloro-2,3,4,5-tetrahydrooxepine-7-carbaldehyde (5.3 g, 34 mmol) in acetonitrile (130 mL) was added potassium thiocyanate (4.96 g, 51 mmol). The reaction mixture was stirred at room temperature for 10 min, then hydrochloric acid (1.0 M, 68 mL, 68 mmol) was added dropwise and the reaction mixture was stirred at room temperature overnight. The mixture was concentrated in vacuo and the residue was diluted with water and extracted with dichloromethane (3×40 mL). The combined organic layers were washed with aqueous sodium hydrogen carbonate and water, dried (Na₂SO₄) and concentrated in vacuo to afford the title compound (6.0 g, 96%) as a light brown oil, which was used directly in the next step without further characterization.

### c) 2-nitro-5,6,7,8-tetrahydrothieno[3,2-b]oxepine

To a mixture of 3-thiocyanato-4,5,6,7-tetrahydrooxepine-2-carbaldehyde (6.0 g, 32.7 mmol) in nitromethane (75 mL) was added DIPEA (4.23 g, 5.72 mL, 32.75 mmol). The reaction mixture was stirred at room temperature for 1.5 h, then concentrated in vacuo. The residue was purified by flash column chromatography (silica, dichloromethane) to afford the title compound (1.65 g, 25%) as a light yellow solid. MS m/z: 200.1 ([M+H]⁺), ESI pos. ¹H NMR (CDCl₃, 400 MHz) δ ppm: 7.52 (s, 1H), 4.04 (d, J=10.1 Hz, 2H), 2.84 (d, J=11.7 Hz, 2H), 2.04 (br d, J=1.6 Hz, 2H), 1.81-1.87 (m, 2H).

### d) N-(5.6,7,8-tetrahydrothieno[3,2-b]oxepin-2-yl)acetamide

To a mixture of 2-nitro-5,6,7,8-tetrahydrothieno[3,2-b]oxepine (1.65 g, 8.28 mmol) in acetic acid (35 mL) was added acetic anhydride (1.69 g, 1.57 mL, 16.6 mmol) and iron powder (2.31 g, 41.4 mmol). The reaction mixture was stirred at 60° C. for 30 min, then filtered through dicalite, washed with ethylacetate and concentrated in vacuo. The residue was purified by flash column chromatography (silica, methanol/dichloromethane 0-5%) to afford the title compound (1.29 g, 74%) as a light yellow solid. MS m/z: 212.2 ([M+H]⁺), ESI pos.

### e) N-[3-(2,6-difluorobenzoyl)-5,6,7,8-tetrahydrothieno[3,2-b]oxepin-2-yl]acetamide

To a solution of N-(5,6,7,8-tetrahydrothieno[3,2-b]oxepin-2-yl)acetamide (817 mg, 3.87 mmol) in 1,2-dichloroethane (15 mL) was added 2,6-difluorobenzoyl chloride (0.724 g, 0.516 mL, 4.06 mmol) and aluminium chloride (0.567 g, 4.25 mmol). The reaction mixture was stirred at 90° C. for 2 h. A further amount of 2,6-difluorobenzoyl chloride (0.345 g, 0.246 mL, 1.93 mmol) and aluminium chloride (0.258 g, 1.93 mmol) were added and the reaction mixture was stirred at 90° C. overnight. The reaction mixture was cooled to room temperature and poured into a mixture of ice and aqueous sodium hydrogen carbonate. The mixture was diluted with ethylacetate and stirred vigrously for 10 min. The phases were separated. The aqueous phase was extracted with ethylacetate (2×70 mL). The combined organic layers were washed with brine, dried (Na₂SO₄) and concentrated in vacuo. The residue was purified by flash column chromatography (silica, ethyl acetate/heptane 0-35%) to afford the title compound (509 mg, 37%) as a yellow solid. MS m/z: 352.1 ([M+H]⁺), ESI pos.

### f) (2-amino-5,6,7,8-tetrahydrothieno[3,2-b]oxepin-3-yl)-(2,6-difluorophenyl)methanone

To a solution of N-[3-(2,6-difluorobenzoyl)-5,6,7,8-tetrahydrothieno[3,2-b]oxepin-2-yl]acetamide (0.515 g, 1.39 mmol) in ethanol (10 mL) was added at 0° C. potassium carbonate (0.385 g, 2.78 mmol). The reaction mixture was stirred for 30 min at 0° C. The ice-bath was removed and the mixture was allowed to warm to room temperature and stirred for additional 5 h. The reaction mixture was poured into ice-water (40 mL). The aqueous phase was extracted with ethylacetate (2×60 mL). The combined organic layers were washed with brine (20 mL), dried (Na₂SO₄) and concentrated in vacuo to afford the title compound (399 mg, 93%) as a yellow solid. MS m/z: 310.1 ([M+H]⁺), ESI pos.

### g) tert-butyl N-[(1S)-2-[[3-(2,6-difluorobenzoyl)-5,6,7,8-tetrahydrothieno[3,2-b]oxepin-2-yl]amino]-1-methyl-2-oxo-ethyl]carbamate

To a mixture of (2S)-2-(tert-Butoxycarbonylamino)propionic acid (0.639 g, 3.31 mmol) in N,N-dimethylformamide (10 mL) was added (2-amino-5,6,7,8-tetrahydrothieno[3,2-b]oxepin-3-yl)-(2,6-difluorophenyl)methanone (512 mg, 1.66 mmol) and DIPEA (1.07 g, 1.45 mL, 8.28 mmol). The reaction mixture was stirred at room temperature for 10 min, then HATU (1.32 g, 3.48 mmol) was added and the mixture was stirred at 40° C. for 22 h. The reaction mixture was concentrated in vacuo and the residue was purified by flash column chromatography (silica, dichloromethane) to afford the title compound (503 mg, 60%) as a yellow solid. MS m/z: 425.1 ([M−isobutene+H]⁺), ESI pos.

### h) (2S)-2-amino-N-[3-(2,6-difluorobenzoyl)-5,6,7,8-tetrahydrothieno[3,2-b]oxepin-2-yl]-propionamide

To a solution of tert-butyl N-[(1S)-2-[[3-(2,6-difluorobenzoyl)-5,6,7,8-tetrahydrothieno[3,2-b]oxepin-2-yl]amino]-1-methyl-2-oxo-ethyl]carbamate (690 mg, 1.44 mmol) in dichloromethane (12 mL) was added at 0° C. trifluoroacetic acid (1.64 g, 1.11 mL, 14.4 mmol). The ice-bath was removed after 5 min and the reaction mixture was allowed to warm to room temperature. The reaction mixture was stirred at room temperature overnight, then diluted with dichloromethane (50 mL) and extracted with saturated aqueous sodium hydrogen carbonate (10 mL). The aqueous phase was extracted with dichloromethane (2×50 mL) and the combined organic layers were dried (Na₂SO₄) and concentrated in vacuo to afford the title compound (482 mg, 84%) as a yellow solid. MS m/z: 381.1 ([M+H]⁺), ESI pos.

### i) (13S)-15-(2,6-difluorophenyl)-13-methyl-3-oxa-9-thia-11,14-diazatricyclo[8.5.0.02,8]pentadeca-1(10),2(8),14-trien-12-one

To a mixture of (2S)-2-amino-N-[3-(2,6-difluorobenzoyl)-5,6,7,8-tetrahydrothieno[3,2-b]oxepin-2-yl]propionamide (482 mg, 1.14 mmol) in toluene (10 mL) was added silica gel (100-200 mesh, 2.4 g) and the orange mixture was stirred at 90° C. for 24 h. The warm reaction mixture (ca 40° C.) was filtered directly on a sintered funnel and the filter cake rinsed with ethylacetate. The filtrate was concentrated in vacuo and purified by flash column chromatography (silica, ethyl acetate/heptane 0-35%) to afford the title compound (240 mg, 56%) as a light yellow solid. MS m/z: 363.3 ([M+H]⁺), ESI pos.

### j) (13S)-15-(2,6-difluorophenyl)-13-methyl-3-oxa-9-thia-11,14-diazatricyclo[8.5.0.02,8]pentadeca-1(10),2(8),14-triene-12-thione

To a mixture of (13S)-15-(2,6-difluorophenyl)-13-methyl-3-oxa-9-thia-11,14-diazatricyclo[8.5.0.02,8]pentadeca-1(10),2(8),14-trien-12-one (40 mg, 0.110 mmol) in acetonitrile (1 mL) was added Lawesson's reagent (26.8 mg, 0.066 mmol). The reaction mixture was stirred in the microwave at 100° C. for 30 min. The reaction mixture was concentrated in vacuo and the residue was purified by flash column chromatography (silica, methanol/dichloromethane 0-5%) to afford the title compound (31 mg, 72%) as a light brown solid. MS m/z: 379.2 ([M+H]⁺), ESI pos.

### Building Block P

### (13S)-15-(2-chloro-6-fluoro-phenyl)-13-methyl-4,7-dioxa-9-thia-11,14-diazatricyclo[8.5.0.02,8]pentadeca-1(10),2(8),14-triene-12-thione

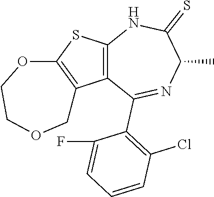 [SMILES: C[C@@H]1N=C(c2c(F)cccc2Cl)c2c(sc3c2COCCO3)NC1=S]

The title compound was obtained in analogy to Building block L, using 3-(2-chloro-6-fluorophenyl)-3-oxopropanenitrile instead of 3-(2,6-difluorophenyl)-3-oxopropanenitrile (in step a), as a light brown oil. MS m/z: 397.1 ([M+H]⁺), ESI pos.

### Building Block Q

### (5S)-3-(2,6-difluorophenyl)-5-methyl-9-thia-4,7-diazatricyclo[8.5.0.02,8]pentadeca-1(10),2(8),3,12-tetraen-6-one

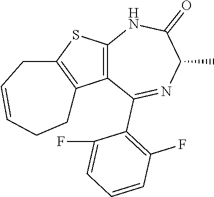 [SMILES: C[C@@H]1N=C(c2c(F)cccc2F)c2c(sc3c2CCC=CC3)NC1=O]

### a) (2-amino-5,8-dihydro-4H-cyclohepta[b]thiophen-3-yl)(2,6-difluorophenyl)methanone

A mixture of a 3-(2,6-difluorophenyl)-3-oxo-propanenitrile (17.27 g, 95.32 mmol), cyclohept-4-en-1-one (10.0 g, 90.79 mmol, CAS #19686-79-4), morpholine (7.91 g, 90.79 mmol) and sulfur (3.05 g, 95.32 mmol) in ethanol (30 mL) was heated to 70° C. in a sealed tube for 14 h. After cooling to room temperature, the reaction mixture was poured into water and extracted with ethyl acetate (3×100 mL). The combined organic layers were dried (Na₂SO₄) and concentrated in vacuo. The residue was purified by flash column chromatography (silica, 0-50% ethyl acetate in petroleum ether) to afford the title compound (12.5 g, 45%) as a light yellow solid. MS m/z: 306.0 ([M+H]⁺), ESI pos.

### b) tert-butyl N-[(1S)-2-[[3-(2,6-difluorobenzoyl)-5,8-dihydro-4H-cyclohepta[b]thiophen-2-yl]amino]-1-methyl-2-oxo-ethyl]carbamate

To a solution of (2S)-2-(tert-butoxycarbonylamino)propionic acid (15.49 g, 81.88 mmol) in N,N-dimethylformamide (250 mL) was added (2-amino-5,8-dihydro-4H-cyclohepta[b]thiophen-3-yl)-(2,6-difluorophenyl)methanone (12.5 g, 40.94 mmol), DIPEA (26.4 g, 204.7 mmol) and HBTU (32.6 g, 85.97 mmol). The reaction mixture was stirred at 40° C. for 14 h. The mixture was diluted with water and the aqueous layer was extracted with ethyl acetate. The combined organic layers were washed with brine, dried (Na₂SO₄) and concentrated in vacuo to afford the title compound (19.5 g, quant.) as a dark brown oil, which was used without further purification. MS m/z: 498.8 ([M+Na]⁺), ESI pos.

### c) (2S)-2-amino-N-[3-(2,6-difluorobenzoyl)-5,8-dihydro-4H-cyclohepta[b]thiophen-2-yl]propanamide

To a mixture of tert-butyl N-[(1S)-2-[[3-(2,6-difluorobenzoyl)-5,8-dihydro-4H-cyclohepta[b]thiophen-2-yl]amino]-1-methyl-2-oxo-ethyl]carbamate (19.5 g, 40.9 mmol) in dichloromethane (300 mL) was added trifluoroacetic acid (61 mL, 401 mmol). The mixture was stirred at room temperature for 6 h, then concentrated in vacuo. The residue was dissolved in dichloromethane (200 mL), treated with saturated aqueous sodium hydrogen carbonate to pH=7. The aqueous layer was extracted with dichloromethane (3×100 mL). The combined organic layers were washed with brine (50 mL), dried (Na₂SO₄) and concentrated in vacuo to afford the title compound (13.5 g, 87%) as a yellow solid. MS m/z: 377.0 ([M+H]⁺), ESI pos.

### d) (5S)-3-(2,6-difluorophenyl)-5-methyl-9-thia-4,7-diazatricyclo[8.5.0.02,8]pentadeca-1(10),2(8),3,12-tetraen-6-one

To a mixture of (2S)-2-amino-N-[3-(2,6-difluorobenzoyl)-5,8-dihydro-4H-cyclohepta[b]thiophen-2-yl]propanamide (13.5 g, 35.9 mmol) in toluene (500 mL) was added silica gel (100-200 mesh, 53.8 g) and 4 Å molecular sieves (30 g). The mixture was stirred at 100° C. under nitrogen for 12 h. The mixture was filtered on a sintered funnel and the filter cake rinsed with ethyl acetate. The filtrate was concentrated in vacuo to afford the title compound (11 g, 90%) as a yellow solid. MS m/z: 358.9 ([M+H]⁺), ESI pos.

### Building Block R

### (5S)-3-(2,6-difluorophenyl)-5-methyl-14-oxa-9-thia-4,7-diazatricyclo[8.5.0.02,8]pentadeca-1(10),2(8),3-trien-6-one

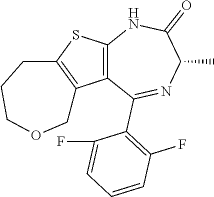 [SMILES: C[C@@H]1N=C(c2c(F)cccc2F)c2c(sc3c2COCCC3)NC1=O]

### a) 2-but-3-enoxyacetic acid

To a suspension of sodium hydride (60% dispersion in mineral oil, 9.27 g, 231.9 mmol) in dry tetrahydrofuran (100 mL) at 0° C. was added 3-buten-1-ol (15.2 g, 210.8 mmol) dropwise over 15 min. The resulting mixture was stirred at 0° C. for 15 min, warmed to 25° C. for 15 min, and then cooled to 0° C. A solution of bromoacetic acid (30.75 g, 221.3 mmol) in tetrahydrofuran (50 mL) was added dropwise. The reaction mixture was warmed to room temperature and stirred for 2 h, before being quenched by addition of water. The volume of tetrahydrofuran was reduced by rotary evaporation. The aqueous layer was acidified with citric acid to pH=3, then extracted with diethyl ether (3×200 mL). The combined organic layers were dried (Na₂SO₄) and concentrated in vacuo to afford the title compound (23.9 g, 87%) as a viscous yellow oil, which was used without further purification in the next step. MS m/z: 129.1 ([M−H]⁺), ESI neg.

### b) 2-but-3-enoxyacetyl chloride

To a mixture of 2-but-3-enoxyacetic acid (23.9 g, 183.4 mmol) in dichloromethane (120 mL) was added oxalyl chloride (27.9 g, 220.1 mmol) and N,N-dimethylformamide (300 mg, 4.1 mmol). The reaction mixture was stirred at 25° C. for 5 h, then concentrated in vacuo to afford the title compound (20.9 g, 77%) as a yellow viscous oil, which was used without further purification in the next step. MS m/z: 149.0 ([M+H]⁺), ESI pos.

### c) 2-but-3-enoxy-N-methoxy-N-methyl-acetamide

To a mixture of 2-but-3-enoxyacetyl chloride (20.9 g, 140.6 mmol) and O,N-dimethylhydroxylamine hydrochloride (17.8 g, 182.8 mmol) in dichloromethane (320 mL), was added dropwise at 0° C. triethylamine (54.9 mL, 393.6 mmol). The reaction mixture was stirred at 0° C. for 4 h, then washed twice with water and brine, dried (Na₂SO₄) and concentrated in vacuo. The residue was purified by flash column chromatography (silica, ethyl acetate in petroleum ether) to afford the title compound (18.0 g, 74%) as a yellow viscous oil. MS m/z: 174.1 ([M+H]⁺), ESI pos.

### d) 1-but-3-enoxybut-3-en-2-one

To a mixture of 2-but-3-enoxy-N-methoxy-N-methyl-acetamide (18.0 g, 103.8 mmol) in anhydrous tetrahydrofuran (180 mL) at −78° C. was added vinylmagnesium bromide (1.0 M, 155 mL, 155 mmol) dropwise and the mixture was stirred at −78° C. for 1 h. The reaction mixture was poured into a stirring pre-cooled mixture of hydrochloric acid (1 M, 250 mL) and dichloromethane (200 mL). The acidic aqueous layer was extracted with dichloromethane (3×100 mL) and the combined organic layers were dried (Na₂SO₄) and concentrated in vacuo to afford the title compound (14.2 g, 98%) as a yellow oil. MS m/z: 141.2 ([M+H]⁺), ESI pos.

### e) 2,3-dihydrooxepin-6-one

To a solution of 1-but-3-enoxybut-3-en-2-one (14.1 g, 100.6 mmol) in anhydrous dichloromethane (5 L), degassed by argon bubbling for 20 min, was added Grubbs' catalyst II (12.8 g, 15.1 mmol). The reaction mixture was heated at 40° C. for 12 h. After cooling to room temperature and concentration in vacuo at 0° C. using and ice-bath, the residue was purified by flash column chromatography (silica, ethyl acetate in petroleum ether) to afford the title compound (7.5 g, 67%) as a dark brown liquid. MS m/z: 113.2 ([M+H]⁺), ESI pos.

### f) triethyl (2,3,4,7-tetrahydrooxepin-6-yloxy)silane

To a solution of 2,3-dihydrooxepin-6-one (6.70 g, 59.7 mmol) and triethylsilane (8.34 g, 71.7 mmol) in dichloromethane (300 mL) was added rhodium(II) acetate (26.4 mg, 0.060 mmol). The reaction mixture was stirred at 40° C. under a nitrogen atmosphere for 8 h. The reaction mixture was quenched by addition of aqueous ammonium chloride (10 wt. %, 2×300 mL). The organic layer was separated, dried (Na₂SO₄) and concentrated in vacuo. The residue was purified by flash column chromatography (silica, ethyl acetate in petroleum ether) to afford the title compound (2.88 g, 21%) as a colorless liquid. MS m/z: 229.3 ([M+H]⁺), ESI pos.

### g) S-(3-oxooxepan-4-yl) ethanethioate

To a solution of triethyl (2,3,4,7-tetrahydrooxepin-6-yloxy)silane (2.88 g, 12.6 mmol) in tetrahydrofuran (200 mL) and water (200 mL) was added at 0° C. N-bromosuccinimide (2.38 g, 13.4 mmol). The reaction mixture was warmed to room temperature and stirred for 1 h, before slow addition of potassium thioacetate (1.18 g, 10.4 mmol). The reaction mixture was stirred for 2 h at room temperature, then concentrated in vacuo. The residue was purified by flash column chromatography (silica, ethyl acetate in petroleum ether) to afford the title compound (900 mg, 92%) as a light yellow viscous oil. MS m/z: 189.1 ([M+H]⁺), ESI pos.

### h) (2-amino-4,6,7,8-tetrahydrothieno[3,2-c]oxepin-3-yl)-(2,6-difluorophenyl)methanone

To a solution of S-(3-oxooxepan-4-yl) ethanethioate (900 mg, 4.78 mmol) in ethanol (15 mL) was added 3-(2,6-difluorophenyl)-3-oxo-propanenitrile (918 mg, 5.07 mmol) and triethylamine (0.72 mL, 5.16 mmol) under a nitrogen atmosphere. The resulting reaction mixture was stirred at 78° C. for 15 h. The mixture was diluted with ethyl acetate (30 mL) and the organic phase washed with brine and water, then concentrated in vacuo. The residue was purified by flash column chromatography (silica, ethyl acetate in petroleum ether) to afford the title compound (803 mg, 54%) as an orange solid. MS m/z: 310.1 ([M+H]⁺), ESI pos.

### i) tert-butyl N-[(1S)-2-[[3-(2,6-difluorobenzoyl)-4,6,7,8-tetrahydrothieno[3,2-c]oxepin-2-yl]amino]-1-methyl-2-oxo-ethyl]carbamate

To a solution of (2S)-2-(tert-butoxycarbonylamino)propionic acid (1.0 g, 5.2 mmol) in dichloromethane (40 mL) was added (2-amino-4,6,7,8-tetrahydrothieno[3,2-c]oxepin-3-yl)-(2,6-difluorophenyl)methanone (803.0 mg, 2.6 mmol), HBTU (1476.72 mg, 3.89 mmol) and DIPEA (671.0 mg, 5.19 mmol). The reaction mixture was stirred at 40° C. for 15 h. The mixture was diluted with ethyl acetate (30 mL) and the organic phase washed with brine, then concentrated in vacuo. The residue was purified by flash column chromatography (silica, ethyl acetate in petroleum ether) to afford the title compound (1.28 g, 87%) as an orange solid. MS m/z: 503.1 ([M+Na]⁺), ESI pos.

### j) (2S)-2-amino-N-[3-(2,6-difluorobenzoyl)-4,6,7,8-tetrahydrothieno[3,2-c]oxepin-2-yl]propanamide

To a solution of tert-butyl N-[(1S)-2-[[3-(2,6-difluorobenzoyl)-4,6,7,8-tetrahydrothieno[3,2-c]oxepin-2-yl]amino]-1-methyl-2-oxo-ethyl]carbamate (1.28 g, 2.67 mmol) in dichloromethane (15 mL) was added hydrochloric acid (4.0 M in dioxane, 3.33 mL, 13.3 mmol). The reaction mixture was stirred at room temperature for 1 h, before being diluted with water. The mixture was extracted with diethyl ether and the organic phase was discarded. The aqueous layer was adjusted to pH=8 by addition of sodium hydrogen carbonate and extracted with dichloromethane (2×150 mL), The combined organic layers were dried (Na₂SO₄) and concentrated in vacuo to afford the title compound (846 mg, 83%) as a yellow solid, which was used without further purification in the next step. MS m/z: 381.1 ([M+H]⁺), ESI pos.

### k) (5S)-3-(2,6-difluorophenyl)-5-methyl-14-oxa-9-thia-4,7-diazatricyclo[8.5.0.02,8]pentadeca-1(10),2(8),3-trien-6-one

To a solution of (2S)-2-amino-N-[3-(2,6-difluorobenzoyl)-4,6,7,8-tetrahydrothieno[3,2-c]oxepin-2-yl]propanamide (846 mg, 2.22 mmol) in toluene (280 mL) was added silica gel (4.51 g) and 4 Å molecular sieves (1.41 g). The reaction mixture was stirred at 100° C. under a nitrogen atmosphere for 8 h. The mixture was filtered on a sintered funnel and the filter cake rinsed with ethyl acetate. The filtrate was concentrated in vacuo and the residue was purified by flash column chromatography (silica, petroleum ether/ethyl acetate 1:2) to afford the title compound (525 mg, 65%) as a yellow solid. MS m/z: 363.1 ([M+H]⁺), ESI pos.

### Building Block S

### (5S)-13-benzyloxy-3-(2,6-difluorophenyl)-5-methyl-11-oxa-9-thia-4,7-diazatricyclo[8.5.0.02,8]pentadeca-1(10),2(8),3-trien-6-one

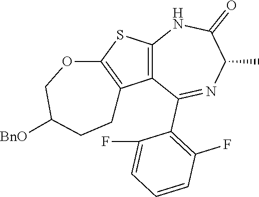 [SMILES: C[C@@H]1N=C(c2c(F)cccc2F)c2c(sc3c2CCC(OCc2ccccc2)CO3)NC1=O]

### a) 1-allyloxypent-4-en-2-ol

To a solution of 2-(allyloxymethyl)oxirane (100.0 g, 876 mmol) in anhydrous tetrahydrofuran (2.0 L) was added copper(I)-iodide (16.7 g, 87.7 mmol) at 0° C., followed by vinyl magnesium bromide (1.0 M in tetrahydrofuran, 877 mL, 877 mmol). The reaction mixture was stirred at 0° C. for 1 h, before being quenched by saturated aqueous ammonium chloride. The aqueous layer was extracted with ethyl acetate (2×1500 mL). The combined organic layers were washed with brine (250 mL), dried (Na₂SO₄) and concentrated in vacuo to afford the title compound (110 g, 88%) as a yellow oil, which was used without further characterization in the next step.

### b) 1-(allyloxymethyl)but-3-enoxymethylbenzene

To a solution of 1-allyloxypent-4-en-2-ol (124.7 g, 877 mmol) in N,N-dimethylformamide (1300 mL) was added sodium hydride (60% in mineral oil, 35.0 g, 877 mmol) at 0° C. The reaction mixture was stirred at 0° C. for 10 min, then benzyl bromide (104 mL, 877 mmol) was added. The solution was stirred at room temperature for 48 h, before being quenched by addition of water (1000 mL) and extracted with ethyl acetate (3×1500 mL). The combined organic layers were washed with brine (1500 mL), dried (Na₂SO₄) and concentrated in vacuo. The residue was purified by flash column chromatography (silica, petroleum ether/ethyl acetate 0-10%) to get the title compound (150 g, 74%) as a yellow oil. MS m/z: 233.2 ([M+H]⁺), ESI pos.

### c) 3-benzyloxy-2,3,4,5-tetrahydrooxepine

To a solution of 1-(allyloxymethyl)but-3-enoxymethylbenzene (50.0 g, 215.2 mmol) in toluene (1000 mL), was added Grubbs II catalyst (5.0 g) under a nitrogen atmosphere. The reaction was stirred at room temperature for 48 h, then sodium hydroxide (12.68 g) in isopropanol (214 mL) was added. The reaction mixture was stirred at 110° C. for 1 h, then concentrated in vacuo. The residue was diluted with ethyl acetate (1 L). The organic phase was washed with water (1000 mL) and concentrated in vacuo. The residue was purified by flash column chromatography (silica, petroleum ether/ethyl acetate 0-10%) to afford the title compound (17 g, 39%) as a yellow oil. MS m/z: 205.2 ([M+H]⁺), ESI pos.

### d) 6-benzyloxyoxepan-3-ol

To a solution of 3-benzyloxy-2,3,4,5-tetrahydrooxepine (6.02 g, 29.5 mmol) in tetrahydrofuran (130 mL) was added borane-dimethylsulfide complex (1.6 mL) at 0° C. The solution was stirred at room temperature for 3 h, then NaBO₃·4H₂O (15.3 g, 92.04 mmol) and water (35 mL, 1.94 mol) were added. The reaction mixture was stirred at room temperature for 16 h, then diluted with ethyl acetate (200 mL). The organic layer was washed with water (3×25 mL), dried (Na₂SO₄) and concentrated in vacuo. The residue was purified by flash column chromatography (silica, dichloromethane/methanol 0-5%) to afford the title compound (3.2 g, 50%) as a yellow oil. MS m/z: 223 ([M+H]⁺), ESI pos.

### e) 6-benzyloxyoxepan-3-one

To a solution of dimethylsulfoxide (6.32 g, 81.0 mmol) in dichloromethane (100 mL) was added oxalyl chloride (4.69 g, 37.0 mmol) dropwise at −78° C. After 30 min, a solution of 6-benzyloxyoxepan-3-ol (6.0 g, 27.0 mmol) in dichloromethane (50 mL) was added into the reaction mixture at −78° C. After 30 min, triethylamine (18.8 mL, 135 mmol) was added and the mixture was stirred at −78° C. for 0.5 h, then at 0° C. for 12 h. The reaction mixture was quenched by addition of aqueous ammonium chloride. The organic phase was washed with brine (50 mL), dried (Na₂SO₄) and concentrated in vacuo. The residue was purified by flash column chromatography (silica, petroleum ether/ethyl acetate 0-60%) to afford the title compound (3.5 g, 58%) as a yellow oil. MS m/z: 221 ([M+H]⁺, ESI pos.

### f) (7-amino-3-benzyloxy-2,3,4,5-tetrahydrothieno[2,3-b]oxepin-6-yl)-(2,6-difluorophenyl) methanone

To a solution of 6-benzyloxyoxepan-3-one (9.0 g, 40.9 mmol) in ethanol (100 mL) was added 3-(2,6-difluorophenyl)-3-oxo-propanenitrile (7.4 g, 40.9 mmol), morpholine (3.56 g, 40.9 mmol) and sulfur (10.5 g, 40.9 mmol). The reaction mixture was stirred at 78° C. for 12 h, then concentrated in vacuo. The residue was purified by flash column chromatography (silica, petroleum ether/ethyl acetate 0-80%) to afford the title compound (10.2 g, 60%) as a brown oil. MS m/z: 416.1 ([M+H]⁺), ESI pos.

### g) tert-butyl N-[(1S)-2-[[3-benzyloxy-6-(2,6-difluorobenzoyl)-2,3,4,5-tetrahydrothieno[2,3-b]oxepin-7-yl]amino]-1-methyl-2-oxo-ethyl]carbamate

To a solution of (7-amino-3-benzyloxy-2,3,4,5-tetrahydrothieno[2,3-b]oxepin-6-yl)-(2,6-difluorophenyl)methanone (6.0 g, 14.4 mmol) in N,N-dimethylformamide (60 mL) was added N-(tert-butoxycarbonyl)-L-alanine (8.2 g, 43.3 mmol), DIPEA (12.6 mL, 72.2 mmol) and HBTU (16.4 g, 43.3 mmol). The reaction mixture was stirred at room temperature for 12 h, before being diluted with ethyl acetate (150 mL). The organic phase was washed with water (3×150 mL), brine (150 mL), dried (Na₂SO₄) and concentrated in vacuo. The residue was purified by flash column chromatography (silica, petroleum ether/ethyl acetate 0-60%) to afford the title compound (7.2 g, 85%) as a yellow solid. MS m/z: 609.2 ([M+Na]⁺), ESI pos.

### h) (2S)-2-amino-N-[3-benzyloxy-6-(2,6-difluorobenzoyl)-2,3,4,5-tetrahydrothieno[2,3-b]oxepin-7-yl]propanamide

To a solution of tert-butyl N-[(1S)-2-[[3-benzyloxy-6-(2,6-difluorobenzoyl)-2,3,4,5-tetrahydrothieno[2,3-b]oxepin-7-yl]amino]-1-methyl-2-oxo-ethyl]carbamate (76.2 g, 129.8 mmol) in dichloromethane (100 mL) was added trifluoroacetic acid (20 mL). The reaction mixture was stirred at room temperature for 16 h, then concentrated in vacuo. The residue was re-dissolved in dichloromethane and saturated aqueous sodium hydrogen carbonate (50 mL) was added. The mixture was extracted with dichloromethane (2×100 mL). The combined organic layers were washed with brine (25 mL), dried (Na₂SO₄) and concentrated in vacuo to afford the title compound (5.50 g, 9%) as a yellow solid. MS m/z: 487.2 ([M+H]⁺), ESI pos.

### h) (5S)-13-benzyloxy-3-(2,6-difluorophenyl)-5-methyl-11-oxa-9-thia-4,7-diazatricyclo[8.5.0.02,8]pentadeca-1(10),2(8),3-trien-6-one

To a solution of (2S)-2-amino-N-[3-benzyloxy-6-(2,6-difluorobenzoyl)-2,3,4,5-tetrahydrothieno[2,3-b]oxepin-7-yl]propanamide (4.50 g, 9.25 mmol) in toluene (90 mL) was added silica gel (10.0 g) and 4 Å molecular sieves (15.0 g). The reaction mixture was stirred at 105° C. for 16 h. The mixture was filtered on a sintered funnel and the filter cake rinsed with dichloromethane (30 mL). The filtrate was concentrated in vacuo and the residue purified by flash column chromatography (silica, dichloromethane/methanol 0-10%) to afford the title compound (2.0 g, 46%) as a yellow solid. MS m/z: 401.1 ([M+Na]⁺), ESI pos.

### Building Block T

### (5S)-3-(2-chloro-6-fluoro-phenyl)-5-methyl-14-oxa-9-thia-4,7-diazatricyclo[8.5.0.02,8]pentadeca-1(10),2(8),3-trien-6-one

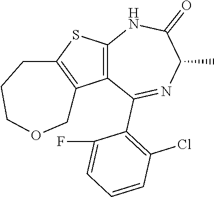 [SMILES: C[C@@H]1N=C(c2c(F)cccc2Cl)c2c(sc3c2COCCC3)NC1=O]

The title compound was obtained in analogy to Building block R using 3-(2-chloro-6-fluoro-phenyl)-3-oxo-propanenitrile (CAS 267881-03-8) instead of 3-(2,6-difluorophenyl)-3-oxo-propanenitrile in step h), as a yellow solid. MS m/z: 378.7 ([M+H]⁺), ESI pos.

### Example 1

### 9-(2,6-difluorophenyl)-3-methyl-18-thia-2,4,5,8-tetrazatetracyclo[8.8.0.02,6.011,17]octadeca-1(10),3,5,8,11(17)-pentaene

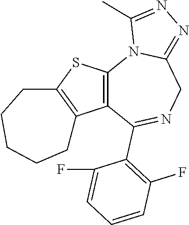 [SMILES: Cc1nnc2n1-c1sc3c(c1C(c1c(F)cccc1F)=NC2)CCCCC3]

To a solution of 3-(2,6-difluorophenyl)-9-thia-4,7-diazatricyclo[8.5.0.02,8]pentadeca-1(10),2(8),3-triene-6-thione (Building block B, 75 mg, 0.207 mmol) in butan-1-ol (2 mL) was added acetohydrazide (46 mg, 0.621 mmol). The reaction mixture was heated using a microwave reactor for 30 min at 100° C., followed by 60 min at 150° C. Then, a second portion of acetohydrazide (30 mg, 0.405 mmol) was added and the mixture was heated for further 75 min at 150° C. in the microwave reactor. The resulting yellow solution was cooled to room temperature and concentrated in vacuo. The residue was dissolved in methanol (2 mL) and purified by preparative HPLC (column Gemini NX 5 u C18 110A, 100×30 mm, 5 micron, eluent: water/acetonitrile) to afford the title compound (57 mg, 71%) as a white powder. MS m/z: 385.2 ([M+H]⁺), ESI pos.

### Example 2

### (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaene

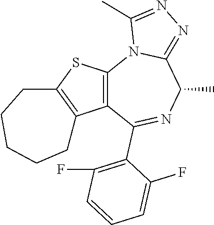 [SMILES: Cc1nnc2n1-c1sc3c(c1C(c1c(F)cccc1F)=N[C@H]2C)CCCCC3]

To a solution of (5S)-3-(2,6-difluorophenyl)-5-methyl-9-thia-4,7-diazatricyclo[8.5.0.02,8]pentadeca-1(10),2(8),3-triene-6-thione (Building block D, 615 mg, 1.63 mmol) in butan-1-ol (15 mL) was added acetohydrazide (672 mg, 8.17 mmol). The reaction mixture was heated using a microwave reactor for 90 min at 150° C. Then, a second portion of acetohydrazide (390 mg, 4.74 mmol) was added and the mixture was heated for further 90 min at 150° C. in the microwave reactor. The resulting yellow solution was cooled to room temperature and concentrated in vacuo. The residue was dissolved in N,N-dimethylformamide (2 mL) and purified by preparative HPLC (Gemini NX 5 u C18 110A, water+0.05% formic acid/acetonitrile, 70:30 to 2:98), followed by chiral HPLC (Reprosil Chiral NR, heptane/(0.01 M ammonium acetate in ethanol) 60:40) to afford the enantiopure (−)-title compound (222 mg, 34%) as a white powder. MS m/z: 399.2 ([M+H]⁺), ESI pos.

### Example 3

### (7S)-3-cyclopropyl-9-(2,6-difluorophenyl)-7-methyl-18-thia-2,4,5,8-tetrazatetracyclo[8.8.0.02,6.011,17]octadeca-1(10),3,5,8,11(17)-pentaene

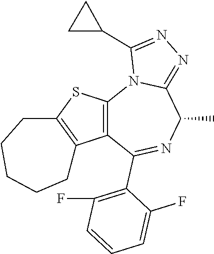 [SMILES: C[C@@H]1N=C(c2c(F)cccc2F)c2c(sc3c2CCCCC3)-n2c(C3CC3)nnc21]

To a solution of (5S)-3-(2,6-difluorophenyl)-5-methyl-9-thia-4,7-diazatricyclo[8.5.0.02,8]pentadeca-1(10),2(8),3-triene-6-thione (Building block D, 65 mg, 0.173 mmol) in butan-1-ol (3 mL) was added cyclopropanecarbohydrazide (86 mg, 0.863 mmol). The reaction mixture was heated using a microwave reactor for 30 min at 150° C., followed by 90 min at 175° C. The resulting yellow solution was cooled to room temperature and concentrated in vacuo. The residue was dissolved in methanol (2 mL) and purified by preparative HPLC (Gemini NX 5 u C18 110A, water+0.05% formic acid/acetonitrile 60:40 to 2:98), followed by chiral HPLC (Reprosil Chiral NR, heptane/(0.01 M ammonium acetate in ethanol) 60:40) to afford the enantiopure (−)-title compound (21 mg, 29%) as a white powder. MS m/z: 425.3 ([M+H]⁺), ESI pos.

### Example 4

### (7S)-9-(2,6-difluorophenyl)-7-methyl-3-pyridazin-3-yl-18-thia-2,4,5,8-tetrazatetracyclo[8.8.0.02,6.011,17]octadeca-1(10),3,5,8,11(17)-pentaene

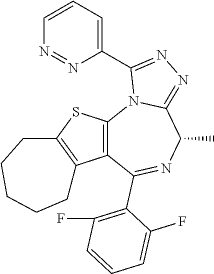 [SMILES: C[C@@H]1N=C(c2c(F)cccc2F)c2c(sc3c2CCCCC3)-n2c(-c3cccnn3)nnc21]

### a) pyridazine-3-carbohydrazide

A suspension of methyl pyridazine-3-carboxylate (10.0 g, 72.4 mmol) in methanol (100 mL) was heated to 60° C., and hydrazine-monohydrate (5.44 g, 5.27 mL, 109 mmol) was added carefully. The brown reaction mixture was stirred for 17 h at 60° C. After cooling to room temperature, the product started to crystallize. Diethyl ether (150 mL) was added and the mixture stirred at 0-4° C. (ice bath). The precipitate was filtered off, washed with diethyl ether (3×100 mL), and dried in vacuo at 45° C. to afford the title compound (8.44 g, 80%) as a light yellow solid. MS m/z: 139.1 ([M+H]⁺), ESI pos. ¹H NMR (CDCl₃, 300 MHz) δ ppm: 9.31 (dd, J=5.0, 1.8 Hz, 1H), 9.03-9.29 (m, 1H), 8.29 (dd, J=8.5, 1.8 Hz, 1H), 7.69 (dd, J=8.5, 5.0 Hz, 1H), 4.17 (br d, J=4.6 Hz, 2H).

### b) (7S)-9-(2,6-difluorophenyl)-7-methyl-3-pyridazin-3-yl-18-thia-2,4,5,8-tetrazatetracyclo[8.8.0.02,6.011,17]octadeca-1(10),3,5,8,11(17)-pentaene

To a solution of (5S)-3-(2,6-difluorophenyl)-5-methyl-9-thia-4,7-diazatricyclo[8.5.0.02,8]pentadeca-1(10),2(8),3-triene-6-thione (Building block D, 85 mg, 0.226 mmol) in butan-1-ol (4 mL) was added pyridazine-3-carbohydrazide (156 mg, 1.13 mmol). The reaction mixture was heated using a microwave reactor for 30 min at 175° C. The resulting yellow solution was cooled to room temperature and concentrated in vacuo. The residue was dissolved in N,N-dimethylformamide (2 mL) and purified by preparative HPLC (Gemini NX 5 u C18 110A, water+0.05% formic acid/acetonitrile 70:30 to 2:98), followed by chiral HPLC (Reprosil Chiral NR, heptane/(0.01 M ammonium acetate in ethanol) 60:40) to afford the enantiopure (−)-title compound (16 mg, 15%) as a light green powder. MS m/z: 463.2 ([M+H]⁺), ESI pos.

### Example 5

### 9-(2,6-difluorophenyl)-3-methyl-14-oxa-18-thia-2,4,5,8-tetrazatetracyclo[8.8.0.02,6.011,17]octadeca-1(10),3,5,8,11(17)-pentaene

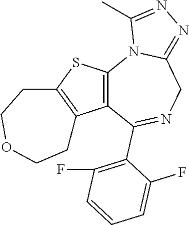 [SMILES: Cc1nnc2n1-c1sc3c(c1C(c1c(F)cccc1F)=NC2)CCOCC3]

To a solution of 3-(2,6-difluorophenyl)-13-oxa-9-thia-4,7-diazatricyclo[8.5.0.02,8]pentadeca-1(10),2(8),3-triene-6-thione (Building block F, 120 mg, 0.329 mmol) in 1-butanol (3 mL) was added acetohydrazide (122 mg, 1.65 mmol). The reaction mixture was heated for 75 min at 130° C. under microwave irradiation, then concentrated in vacuo. The residue was purified by preparative HPLC (Gemini NX 5 u C18 110A, 0.05% formic acid in water/acetonitrile) to afford the title compound (67 mg, 52%) as an off-white lyophilized powder. MS m/z: 387.2 ([M+H]⁺), ESI pos.

### Example 6

### (7S)-9-(2,6-difluorophenyl)-7-methyl-18-thia-2,4,5,8-tetrazatetracyclo[8.8.0.02,6.011,17]octadeca-1(10),5,8,11(17)-tetraen-3-one

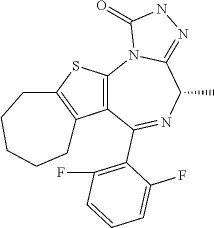 [SMILES: C[C@@H]1N=C(c2c(F)cccc2F)c2c(sc3c2CCCCC3)-n2c1n[nH]c2=O]

### a) (5S)-3-(2,6-difluorophenyl)-5-methyl-9-thia-4,7-diazatricyclo[8.5.0.02,8]pentadeca-1(10),2(8),3-trien-6-one hydrazone

To a solution of (5S)-3-(2,6-difluorophenyl)-5-methyl-9-thia-4,7-diazatricyclo[8.5.0.02,8]pentadeca-1(10),2(8),3-triene-6-thione (Building block D, 150 mg, 0.398 mmol) in 2-propanol (0.7 mL) and tetrahydrofuran (3.5 mL) was added hydrazine hydrate (40 mg, 39 mL, 0.797 mmol). The reaction mixture was stirred for 1 h at room temperature, then concentrated in vacuo. The residue was dissolved in acetonitrile (5 mL) and the resulting solution was concentrated in vacuo. The resulting crude product was used in the next step without further purification (148 mg, 99%). MS m/z: 375.1 ([M+H]⁺), ESI pos.

### b) (7S)-9-(2,6-difluorophenyl)-7-methyl-18-thia-2,4,5,8-tetrazatetracyclo[8.8.0.02,6.011,17]octadeca-1(10),5.8.11(17)-tetraen-3-one

To a solution of (5S)-3-(2,6-difluorophenyl)-5-methyl-9-thia-4,7-diazatricyclo[8.5.0.02,8]pentadeca-1(10),2(8),3-trien-6-one hydrazone (148 mg, 0.395 mmol) in tetrahydrofuran (5 mL) was added carbonyldiimidazole (CDI, 77 mg, 0.474 mmol). The reaction mixture was stirred for 4 h at 70° C., before being cooled to room temperature. The mixture was diluted with tert-butylmethyl ether (15 mL) and the organic layer was washed with aqueous sodium carbonate (1.0 M, 15 mL), water (15 mL) and brine (15 mL). The aqueous layers were extracted with tert-butylmethyl ether (15 mL). The combined organic layers were dried (MgSO₄) and concentrated in vacuo. The residue was purified by flash chromatography (silica, heptane/ethyl acetate, 90:10 to 60:40), followed by chiral preparative HPLC (Reprosil Chiral NR, heptane/(0.01 M ammonium acetate in ethanol) 70:30) to afford the enantiopure (−)-title compound (64 mg, 40%) as a white solid. MS m/z: 401.2 ([M+H]⁺), ESI pos.

### Example 7

### (8S)-10-(2,6-difluorophenyl)-8-methyl-19-thia-2,6,9-triazatetracyclo[9.8.0.02,7.012,18]nonadeca-1(11),3,6,9,12(18)-pentaen-5-one

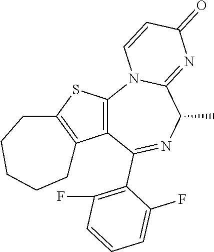 [SMILES: C[C@@H]1N=C(c2c(F)cccc2F)c2c(sc3c2CCCCC3)-n2ccc(=O)nc21]

### a) (5S)-3-(2,6-difluorophenyl)-5-methyl-9-thia-4,7-diazatricyclo[8.5.0.02,8]pentadeca-1(10),2(8),3-trien-6-imine

A solution of (5S)-3-(2,6-difluorophenyl)-5-methyl-9-thia-4,7-diazatricyclo[8.5.0.02,8] pentadeca-1(10),2(8),3-triene-6-thione (Building block D, 112 mg, 0.297 mmol) in ammonia (7.0 M in methanol, 1.27 mL, 8.92 mmol) was stirred at 60° C. for 24 h, then concentrated in vacuo. The residue was purified by flash column chromatography (silica, 0-80% ethyl acetate in heptane) to afford the title compound (99 mg, 90%) as an off-white solid. MS m/z: 360.2 ([M+H]⁺), ESI pos.

### b) (8S)-10-(2,6-difluorophenyl)-8-methyl-19-thia-2,6,9-triazatetracyclo[9.8.0.02,7.012,18]nonadeca-1(11),3,6,9,12(18)-pentaen-5-one

To a solution of (5S)-3-(2,6-difluorophenyl)-5-methyl-9-thia-4,7-diazatricyclo[8.5.0.02,8]pentadeca-1(10),2(8),3-trien-6-imine (97 mg) and ethanol (2 mL) was added at 23° C. ethyl propiolate (132 mg, 137 μl). The yellow reaction solution was stirred at 60° C. for 16 h, then concentrated in vacuo. The residue was purified by chiral HPLC (Chiralcel OD, 5% methanol in heptane) to afford the enantiopure (−)-title compound (31 mg, 28%) as a brown foam. MS m/z: 412.2 ([M+H]⁺), ESI pos.

### Example 8

### (7S)-9-(2,6-difluorophenyl)-15,15-difluoro-3,7-dimethyl-18-thia-2,4,5,8-tetrazatetracyclo[8.8.0.02,6.011,17]octadeca-1(10),3,5,8,11(17)-pentaene

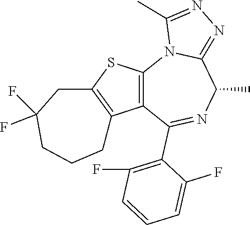 [SMILES: Cc1nnc2n1-c1sc3c(c1C(c1c(F)cccc1F)=N[C@H]2C)CCCC(F)(F)C3]

To a solution of (5S)-3-(2,6-difluorophenyl)-12,12-difluoro-5-methyl-9-thia-4,7-diazatricyclo[8.5.0.02,8]pentadeca-1(10),2(8),3-triene-6-thione (Building block E, 275 mg, 0.667 mmol) in butan-1-ol (15 mL) was added acetohydrazide (274 mg, 3.33 mmol). The reaction mixture was heated at 150° C. for 2.5 h in a microwave reactor. The resulting brown solution was cooled to room temperature and concentrated in vacuo. The residue was purified by chiral HPLC (Reprosil Chiral NR, heptane/(0.01 M ammonium acetate in ethanol) 60:40) to afford the enantiopure (−)-title compound (44 mg, 15%) as a white solid. MS m/z: 435.2 ([M+H]⁺), ESI pos.

### Example 9

### (7S,16RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-16-ol

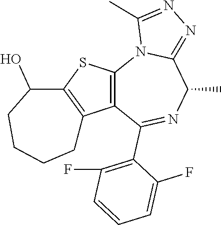 [SMILES: Cc1nnc2n1-c1sc3c(c1C(c1c(F)cccc1F)=N[C@H]2C)CCCCC3O]

### a) (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-16-one

To a solution of (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaene (330 mg, 0.828 mmol) in acetonitrile (8 mL) and water (8 mL) was added potassium persulfate (448 mg, 1.66 mmol) and copper (II) sulfate pentahydrate (414 mg, 1.66 mmol). The reaction mixture was stirred at 70° C. for 4 h and at 23° C. for another 24 h. The mixture was diluted with dichloromethane (30 mL) and washed with aqueous sodium thiosulfate (1.0 M, 10 mL). The aqueous layer was extracted with dichloromethane (2×30 mL). The combined organic layers were dried (MgSO₄) and concentrated in vacuo. The resulting yellow oil was purified by flash column chromatography (silica, 0-80% ethyl acetate in heptane) to afford the title compound (221 mg, 58%) as a white foam. MS m/z: 413.3 ([M+H]⁺), ESI pos.

### b) (7S,16RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-16-ol

To a solution of (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-16-one (218 mg, 0.529 mmol) in methanol (10 mL) was added at 0-4° C. (ice bath) lithium borohydride (34.5 mg, 1.59 mmol). The reaction mixture was stirred at room temperature for 3 h, before bing quenched by addition of ice-water (10 mL) and diluted with dichloromethane (40 mL) and semi-saturated aqueous ammonium chloride (20 mL). The aqueous phase was extracted with dichloromethane (2×20 mL). The combined organic layers were dried (Na₂SO₄) and concentrated in vacuo to afford the title compound (226 mg, 88%) as a white foam. MS m/z: 415.3 ([M+H]⁺), ESI pos.

### Example 10

### (7S,16RS)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene

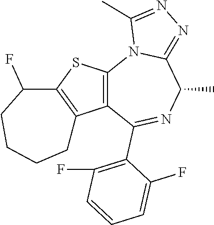 [SMILES: Cc1nnc2n1-c1sc3c(c1C(c1c(F)cccc1F)=N[C@H]2C)CCCCC3F]

To a solution of (7S,16RS)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-16-ol (226 mg, 0.545 mmol) in dichloromethane (8 mL) was added at 0-4° C. (ice bath) diethylaminosulfur trifluoride (146 mg, 120 μl, 0.818 mmol). The reaction mixture was stirred at room temperature for 16 h, before being diluted with water (10 mL). The aqueous phase was extracted with dichlormethane (2×15 mL) and combined organic layers were dried (Na₂SO₄) and concentrated in vacuo. The residue was purified by chiral HPLC (Reprosil Chiral NR, heptane/ethanol 60:40) to afford the enantiopure (−)-title compound (92 mg, 36.5%) as a light brown foam. MS m/z: 417.3 ([M+H]⁺), ESI pos.

### Example 11

### (7S,16R)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene

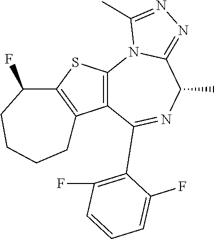 [SMILES: Cc1nnc2n1-c1sc3c(c1C(c1c(F)cccc1F)=N[C@H]2C)CCCC[C@H]3F]

A sample of (7S,16RS)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene (86 mg, 0.206 mmol) was separated by chiral SFC (CHIRALPAK® IC (Daicel), 30% methanol) to afford the enantiopure (−)-title compound (24 mg, 27%) as a light brown foam. MS m/z: 417.3 ([M+H]⁺), ESI pos. ¹H NMR (DMSO-d₆, 300 MHz) δ ppm: 7.55 (tt, J=8.5, 6.5 Hz, 1H), 7.17 (br s, 2H), 5.81-6.05 (m, 1H), 4.40 (q, J=6.7 Hz, 1H), 2.62 (s, 3H), 2.30-2.42 (m, 1H), 2.08-2.19 (m, 1H), 1.91-2.07 (m, 2H), 1.88 (d, J=6.6 Hz, 3H), 1.64-1.85 (m, 2H), 1.28-1.44 (m, 1H), 1.03-1.18 (m, 1H).

### Example 12

### (7S,16S)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene

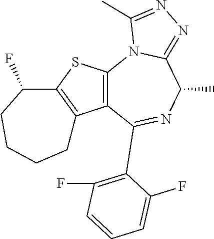 [SMILES: Cc1nnc2n1-c1sc3c(c1C(c1c(F)cccc1F)=N[C@H]2C)CCCC[C@@H]3F]

In analogy to experiment of example 11, the enantiopure (−)-title compound (32 mg, 33%) was obtained as a light brown foam. MS m/z: 417.3 ([M+H]⁺), ESI pos. ¹H NMR (DMSO-d₆, 300 MHz) δ ppm: 7.54 (tt, J=8.4, 6.6 Hz, 1H), 7.16 (br s, 2H), 5.81-6.04 (m, 1H), 4.45 (q, J=6.6 Hz, 1H), 2.61 (s, 3H), 2.36-2.47 (m, 1H), 2.02-2.18 (m, 2H), 1.90-2.01 (m, 2H), 1.87 (d, J=6.6 Hz, 3H), 1.53-1.71 (m, 1H), 1.29-1.49 (m, 2H).

NOTE: The assignment of the absolute configuration at C-16 (see name) in Example 11 and Example 12 was done arbitrarily, both in drawing and naming of the compounds.

### Example 13

### (7S,15RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-ol

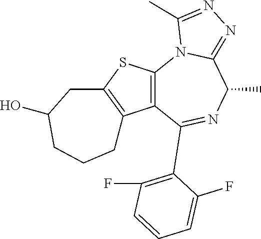 [SMILES: Cc1nnc2n1-c1sc3c(c1C(c1c(F)cccc1F)=N[C@H]2C)CCCC(O)C3]

To a solution of (7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-one (Building block G, 146 mg, 0.354 mmol) in methanol (3 mL) was added lithium borohydride (23.1 mg, 1.06 mmol) under ice cooling. The reaction mixture was stirred at room temperature for 3 h, before being quenched by addition of ice-water. The mixture was partitioned between dichloromethane and aqueous ammonium chloride. The combined organic layers were dried (Na₂SO₄) and concentrated in vacuo to afford the title compound (152 mg, 98%) as a light yellow foam. MS m/z: 415.2 ([M+H]⁺), ESI pos.

### Example 14

### (7S,14S)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene

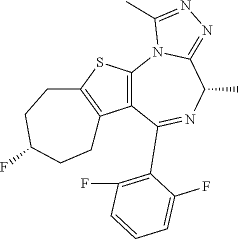 [SMILES: Cc1nnc2n1-c1sc3c(c1C(c1c(F)cccc1F)=N[C@H]2C)CC[C@H](F)CC3]

### a) (7S,14RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-ol

To a solution of (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-14-one (Building block H, 1.60 g, 3.88 mmol) in methanol (40 mL) was added at 0-4° C. (ice bath) lithium borohydride (253 mg, 11.6 mmol). The reaction mixture was stirred at room temperature for 3 h, before being quenched by addition of ice-water (20 mL). The mixture was partitioned between dichloromethane (50 mL) and semi-saturated aqueous ammonium chloride (20 mL). The aqueous phase was extracted with dichloromethane (2×50 mL). The combined organic layers were washed with water (50 mL) and brine (50 mL), dried (Na₂SO₄) and concentrated in vacuo to afford the title compound (1.66 g, quant.) as a white foam, which was used in the following step without further purification. MS m/z: 415.2 ([M+H]⁺), ESI pos.

### b) (7S,14S)-9-(2,6-difluorophenyl)-14-fluoro-3,7-dimethyl-18-thia-2,4,5,8-tetrazatetracyclo[8.8.0.02,6.011,17]octadeca-1(10),3.5.8.11(17)-pentaene

To a solution of (7S,14RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-ol (134 mg, 0.323 mmol) in dichloromethane (3 mL) was added at 0-4° C. (ice bath) diethylaminosulfur trifluoride (DAST, 78.2 mg, 64.1 μl, 0.485 mmol) and the reaction mixture was stirred at room temperature for 16 h. The reaction was quenched by addition of water (10 mL), then diluted with dichloromethane (10 mL). The aqueous phase was extracted with dichloromethane (2×30 mL). The combined organic layers were washed with water (20 mL) and brine (20 mL), dried (Na₂SO₄) and concentrated in vacuo. The residue was purified by preparative chiral HPLC (Reprosil Chiral NR, heptane/(0.01 M ammonium acetate in ethanol) 60:40) to afford the enantiopure (−)-title compound (12 mg, 9%) as a white amorphous solid. MS m/z: 417.3 ([M+H]⁺), ESI pos. ¹H NMR (DMSO-d₆, 300 MHz) δ ppm: 7.56 (tt, J=8.5, 6.6 Hz, 1H), 7.18 (br s, 2H), 4.77 (dtt, J=45.7, 9.3, 3.4 Hz, 1H), 4.38 (q, J=6.4 Hz, 1H), 2.99 (dd, J=16.1, 8.1 Hz, 1H), 2.64-2.78 (m, 1H), 2.60 (s, 3H), 2.17-2.25 (m, 2H), 2.05-2.17 (m, 1H), 1.87 (d, J=6.6 Hz, 3H), 1.50-1.81 (m, 2H), 1.03-1.19 (m, 1H).

### Example 15

### (7S,14R)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene

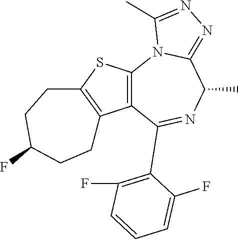 [SMILES: Cc1nnc2n1-c1sc3c(c1C(c1c(F)cccc1F)=N[C@H]2C)CC[C@@H](F)CC3]

In analogy to experiment of example 14 b, the enantiopure (−)-title compound (5 mg, 4%) was obtained as a white amorphous solid. MS m/z: 417.3 ([M+H]⁺), ESI pos. ¹H NMR (DMSO-d₆, 300 MHz) δ ppm: 7.55 (tt, J=8.4, 6.6 Hz, 1H), 7.17 (br s, 2H), 4.80 (dtt, J=45.5, 6.4, 3.4 Hz, 1H), 4.39 (q, J=6.8 Hz, 1H), 3.37-3.47 (m, 1H), 2.95-3.09 (m, 1H), 2.70-2.80 (m, 1H), 2.59 (s, 3H), 1.89-2.02 (m, 2H), 1.87 (d, J=6.6 Hz, 3H), 1.54-1.65 (m, 1H), 1.45-1.53 (m, 1H), 1.26-1.33 (m, 1H).

NOTE: The assignment of the absolute configuration at C-14 (see name) in Example 14 and Example 15 was done arbitrarily, both in drawing and naming of the compounds.

### Example 16

### (7S,15R)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene

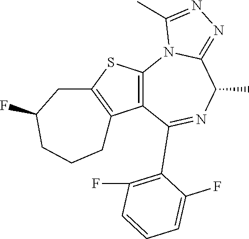 [SMILES: Cc1nnc2n1-c1sc3c(c1C(c1c(F)cccc1F)=N[C@H]2C)CCC[C@@H](F)C3]

### a) (7S,15RS)-9-(2,6-difluorophenyl)-3,7-dimethyl-18-thia-2,4,5,8-tetrazatetracyclo[8.8.0.02,6.011,17]octadeca-1(10),3.5.8.11(17)-pentaen-15-ol

To a solution of (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-15-one (Building block G, 146 mg, 0.354 mmol) in methanol (3 mL) was added at 0-4° C. (ice bath) lithium borohydride (23 mg, 1.1 mmol) and the reaction mixture was stirred at 23° C. for 3 h. The reaction was quenched by addition of ice-water (5 mL), then diluted with dichloromethane (10 mL) and semi-saturated aqueous ammonium chloride (50 mL). The aqueous phase was extracted with dichloromethane (2×30 mL). The combined organic layers were washed with water (15 mL) and brine (15 mL), dried (Na₂SO₄) and concentrated in vacuo to afford the title compound (152 mg, 98%) as a light yellow foam, which was used in the following step without further purification. MS m/z: 415.2 ([M+H]⁺), ESI pos.

### b) (7S,15R)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene

To a solution of (7S,15RS)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-15-ole (398 mg, 0.960 mmol) in dichloromethane (8 mL) was added at 0-4° C. (ice bath) diethylaminosulfur trifluoride (DAST, 224 mg, 183 μl, 1.25 mmol). The reaction mixture was stirred at room temperature for 2 h, before being quenched by addition of water (20 mL). The mixture was diluted with dichloromethane (20 mL), and aqueous phase was extracted with dichloromethane (2×20 mL). The combined organic layers were washed with water (40 mL) and brine (40 mL), dried (Na₂SO₄) and concentrated in vacuo. The residue was purified by chiral preparative SFC (CHIRALPAK® AS-H (Daicel), 25% ethanol) to afford the enantiopure (−)-title compound (64 mg, 16%) as a white amorphous solid. MS m/z: 417.3 ([M+H]⁺), ESI pos. ¹H NMR (DMSO-d₆, 300 MHz) δ ppm: 7.54 (tt, J=8.4, 6.6 Hz, 1H), 7.16 (br s, 2H), 4.61 (dtt, J=45.9, 9.5, 3.2 Hz, 1H), 4.38 (q, J=7.1 Hz, 1H), 3.32-3.40 (m, 1H), 3.13-3.28 (m, 1H), 2.60 (s, 3H), 2.29-2.41 (m, 1H), 1.97-2.25 (m, 2H), 1.87 (d, J=7.3 Hz, 3H), 1.76-1.96 (m, 1H), 1.31-1.50 (m, 1H), 0.88-1.07 (m, 1H).

### Example 17

### (7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene

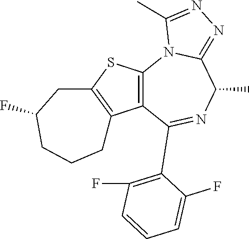 [SMILES: Cc1nnc2n1-c1sc3c(c1C(c1c(F)cccc1F)=N[C@H]2C)CCC[C@H](F)C3]

In analogy to experiment of example 16 b, the enantiopure (−)-title compound (87 mg, 22%) was obtained as a white amorphous solid. MS m/z: 417.3 ([M+H]⁺), ESI pos. ¹H NMR (DMSO-d₆, 300 MHz) δ ppm: 7.54 (tt, J=8.5, 6.6 Hz, 1H), 7.16 (br s, 2H), 4.76 (dtt, J=45.9, 10.5, 3.4 Hz, 1H), 4.37 (q, J=6.6 Hz, 1H), 3.25-3.30 (m, 1H), 3.15-3.25 (m, 1H), 2.59 (s, 3H), 2.29-2.42 (m, 1 H), 1.99-2.20 (m, 2H), 1.91-1.99 (m, 1H), 1.87 (d, J=6.6 Hz, 3H), 1.30-1.44 (m, 1H), 1.18-1.30 (m, 1H).

NOTE: The assignment of the absolute configuration at C-15 (see name) in Example 16 and Example 17 was done arbitrarily, both in drawing and naming of the compounds.

### Example 18

### 9-(2,6-difluorophenyl)-3-methyl-16-oxa-18-thia-2,4,5,8-tetrazatetracyclo[8.8.0.02,6.011,17]octadeca-1(10),3,5,8,11(17)-pentaene

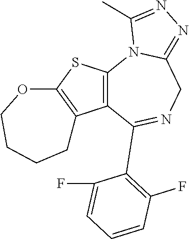 [SMILES: Cc1nnc2n1-c1sc3c(c1C(c1c(F)cccc1F)=NC2)CCCCO3]

To a solution of 3-(2,6-difluorophenyl)-11-oxa-9-thia-4,7-diazatricyclo[8.5.0.02,8] pentadeca-1(10),2(8),3-trien-6-thione (Building block I, 140 mg, 0.230 mmol) in 1-butanol (4 mL) was added acethydrazide (94.9 mg, 1.15 mmol). The reaction mixture was stirred in the microwave at 130° C. for 2 h, before being concentrated in vacuo. The residue was purified by flash chromatography (silica, 0-8% methanol in dichloromethane) to afford the title compound (27 mg, 30%) as a light yellow solid. MS m/z: 387.2 ([M+H]⁺), ESI pos.

### Example 19

### (7S)-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaene

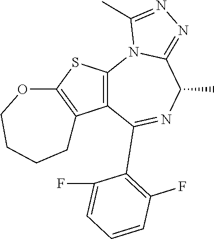 [SMILES: Cc1nnc2n1-c1sc3c(c1C(c1c(F)cccc1F)=N[C@H]2C)CCCCO3]

To a solution of (5S)-3-(2,6-difluorophenyl)-5-methyl-11-oxa-9-thia-4,7-diazatricyclo[8.5.0.02,8]pentadeca-1(10),2(8),3-triene-6-thione (Building block J, 106 mg, 0.224 mmol) in 1-butanol (2 mL) was added acethydrazide (92.2 mg, 1.12 mmol). The reaction mixture was stirred in the microwave at 130° C. for 60 min. A further amount of acethydrazide (92.2 mg, 1.12 mmol) was added and microwave irradiation was continued for another 60 min. The reaction mixture was concentrated in vacuo and the residue was purified by flash column chromatograpy (silica, 0-6% methanol in dichloromethane), followed by chiral preparative SFC (Daicel CHIRALPAK® OZ—H, 25% methanol) to afford the title compound (17 mg, 19%) as a white solid. MS m/z: 401.3 ([M+H]⁺), ESI pos.

### Example 20

### (7S)-9-(2,6-difluorophenyl)-4,7-dimethyl-18-thia-2,3,5,8-tetrazatetracyclo[8.8.0.02,6.011,17]octadeca-1(10),3,5,8,11(17)-pentaene

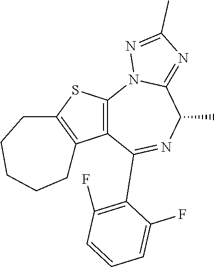 [SMILES: Cc1nc2n(n1)-c1sc3c(c1C(c1c(F)cccc1F)=N[C@H]2C)CCCCC3]

### a) (5S)-7-amino-3-(2,6-difluorophenyl)-5-methyl-9-thia-4,7-diazatricyclo[8.5.0.02,8]pentadeca-1(10),2(8),3-trien-6-one

To a solution of (5S)-3-(2,6-difluorophenyl)-5-methyl-9-thia-4,7-diazatricyclo[8.5.0.02,8]pentadeca-1(10),2(8),3-trien-6-one (Building block C, 168 mg, 0.466 mmol) in N,N-dimethyformamide (5 mL) was added at 0-4° C. (ice bath) 0-(diphenylphosphinyl)hydroxylamine (134 mg, 0.559 mmol) and cesium carbonate (228 mg, 0.699 mmol). The reaction mixture was stirred for 3 h at 0-4° C., before being quenched by addition of water (20 mL). The mixture was extracted with ethyl acetate (2×40 mL). The combined organic layers were washed with water (20 mL) and brine (20 mL), dried (Na₂SO₄) and concentrated in vacuo. The residue was purified by flash column chromatography (silica, 0-30% ethyl acetate in heptane) to afford the title compound (169 mg, 95%) as a light yellow foam. MS m/z: 376.2 ([M+H]⁺), ESI pos.

### b) (7S)-9-(2,6-difluorophenyl)-4,7-dimethyl-18-thia-2,3,5,8-tetrazatetracyclo[8.8.0.02,6.011,17]octadeca-1(10),3,5,8,11(17)-pentaene

A mixture of (5S)-7-amino-3-(2,6-difluorophenyl)-5-methyl-9-thia-4,7-diazatricyclo[8.5.0.02,8]pentadeca-1(10),2(8),3-trien-6-one (169 mg, 0.450 mmol), thioacetamide (1.69 g, 22.5 mmol) and zinc chloride (123 mg, 0.900 mmol) was heated to 120° C. for 3 h to form a brown liquid. The mixture was allowed to cool to room temperature and the crude reaction was purified by flash column chromatography (silica, heptane/ethyl acetate 1:1 (v/v)), followed by chiral SFC (Daicel CHIRALPAK® IG, 20% methanol) to afford the enantiopure (−)-title compound (6 mg, 3%) as a white foam. MS m/z: 399.2 ([M+H]⁺), ESI pos.

### Example 21

### (7S)-9-(2,6-difluorophenyl)-3,7-dimethyl-13,16-dioxa-18-thia-2,4,5,8-tetrazatetracyclo[8.8.0.02,6.011,17]octadeca-1(10),3,5,8,11(17)-pentaene

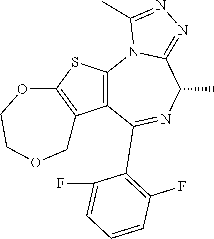 [SMILES: Cc1nnc2n1-c1sc3c(c1C(c1c(F)cccc1F)=N[C@H]2C)COCCO3]

To a solution of (13S)-15-(2,6-difluorophenyl)-13-methyl-4,7-dioxa-9-thia-11,14-diazatricyclo[8.5.0.02,8] pentadeca-1(10),2(8),14-triene-12-thione (Building block L, 590 mg, 1.55 mmol) in 1-butanol (10 mL) was added acethydrazide (638 mg, 7.75 mmol). The reaction mixture was stirred with microwave irradiation at 130° C. for 60 min. A further amount of acethydrazide (638 mg, 7.75 mmol) was added and the reaction mixture was stirred in the microwave at 130° C. for another 60 min. The reaction mixture was concentrated in vacuo and the residue was purified by flash column chromatography (silica, 0-6% methanol in dichloromethane), followed by chiral SFC (Daicel CHIRALPAK® IC, 20-40% methanol) to afford the enantiopure (−)-title compound (74 mg, 12%) as a light yellow solid. MS m/z: 403.3 ([M+H]⁺), ESI pos.

### Example 22

### azetidin-1-yl-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

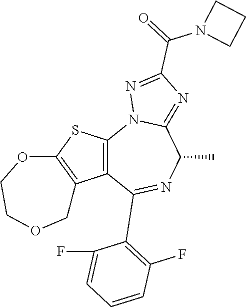 [SMILES: C[C@@H]1N=C(c2c(F)cccc2F)c2c(sc3c2COCCO3)-n2nc(C(=O)N3CCC3)nc21]

### a) (13S)-11-amino-15-(2,6-difluorophenyl)-13-methyl-4,7-dioxa-9-thia-11,14-diazatricyclo[8.5.0.02,8]pentadeca-1(10),2(8),14-trien-12-one

To a solution of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K, 1.5 g, 4.3 mmol) and O-(diphenylphosphinyl)hydroxylamine (1.2 g, 5.1 mmol) in N,N-dimethylformamide (20 mL) was added cesium carbonate (2.1 g, 6.4 mmol) at 0° C. The mixture was stirred at room temperature for 4 h, then diluted with ethyl acetate (100 mL). The organic layer was washed with water (3×50 mL), dried (Na₂SO₄) and concentrated in vacuo. The residue was purified by flash column chromatography (silica, petroleum ether/ethyl acetate, 5:1 to 1:1) to afford the title compound (770 mg, 52%) as a light yellow solid. MS m/z: 380.0 ([M+H]⁺), ESI pos.

### b) ethyl (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylate

To a solution of (13S)-11-amino-15-(2,6-difluorophenyl)-13-methyl-4,7-dioxa-9-thia-11,14-diazatricyclo[8.5.0.02,8]pentadeca-1(10),2(8),14-trien-12-one (770 mg, 2.0 mmol) in toluene (30 mL) was added ethyl 2-ethoxy-2-imino-acetate (736 mg, 2.5 mmol) and acetic acid (244 mg, 0.232 mL, 4.0 mmol). The mixture was stirred at 100° C. under nitrogen for 22 h, before being allowed to warm to room temperature. The mixture was diluted with ethyl acetate (80 mL) and washed with water (2×20 mL). The aqueous layer was further extracted with ethyl acetate (3×50 mL). The combined organic layers were washed with brine (50 mL), dried (Na₂SO₄) and concentrated in vacuo. The residue was purified by flash column chromatography (silica, petroleum ether/ethyl acetate, 10:1 to 1:8) to afford the title compound (90% pure, 450 mg, 44%) as a yellow solid. MS m/z: 461.0 ([M+H]⁺), ESI pos.

### c) (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylic acid

To a solution of ethyl (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylate (450 mg, 0.88 mmol) in methanol (10 mL) and water (5 mL) was added lithium hydroxide (63.0 mg, 2.6 mmol). The mixture was stirred at room temperature for 5 h. The mixture was diluted with water (30 mL) and freeze-dried to give a crude which was treated with dichloromethane (50 mL), filtrated and concentrated in vacuo to afford the title compound (290 mg, 61%) as a light yellow solid. MS m/z: 433.0 ([M+H]⁺), ESI pos.

### d) azetidin-1-yl-[(7S)-9-(2,6-difluorophenyl)-7-methyl-13,16-dioxa-18-thia-2,3,5,8-tetrazatetracyclo[8.8.0.02,6.011,17]octadeca-1(10),3.5.8.11(17)-pentaen-4-yl]methanone

To a solution of (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylic acid (50.0 mg, 0.1 mmol) and azetidine (13.2 mg, 0.2 mmol) in dichloromethane (5 mL) was added propylphosphonic anhydride (147.1 mg, 0.2 mmol) and DIPEA (44.8 mg, 0.3 mmol). The reaction mixture was stirred at room temperature for 3 h, before being diluted with dichloromethane (20 mL). The organic layer was washed with water (10 mL). The aqueous layer was further extracted with dichloromethane (3×10 mL). The combined organic layers were washed with brine (50 mL), dried (Na₂SO₄) and concentrated in vacuo. The residue was purified by flash column chromatography (silica, petroleum ether/ethyl acetate, 5:1 to 1:5), followed by chiral SFC (Chiralpak AD-H, 35% methanol) to afford the enantiopure (−)-title compound (19.4 mg, 36%) as a white solid. MS m/z: 472.0 ([M+H]⁺), ESI pos.

### Example 23

### azetidin-1-yl-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

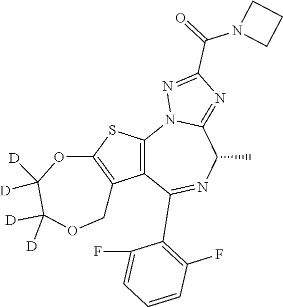 [SMILES: [2H]C1([2H])OCc2c(sc3c2C(c2c(F)cccc2F)=N[C@@H](C)c2nc(C(=O)N4CCC4)nn2-3)OC1([2H])[2H]]

### a) (13S)-11-amino-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one

To a solution of (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (Building block M, 202 mg, 0.550 mmol) and O-(diphenylphosphinyl)hydroxylamine (154 mg, 0.660 mmol) in N,N-dimethylformamide (10 mL) was added cesium carbonate (268 mg, 0.820 mmol) at 0° C. The reaction mixture was stirred at room temperature for 4 h, before being diluted with ethyl acetate (50 mL). The mixture was washed with water (3×20 mL), dried (Na₂SO₄) and concentrated in vacuo. The residue was purified by flash column chromatography (silica, 0-100% ethyl acetate in petroleum ether) to afford the title compound (125 mg, 54%) as a light yellow solid. MS m/z: 383.7 ([M+H]⁺), ESI pos.

### b) ethyl (7S)-14,14,15,15-tetradeuterio-9-(2,6-difluorophenyl)-7-methyl-13,16-dioxa-18-thia-2,3,5,8-tetrazatetracyclo[8.8.0.02,6.011,17]-octadeca-1(10),3,5,8,11(17)-pentaene-4-carboxylate

To a solution of (13S)-11-amino-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (125 mg, 0.33 mmol) in toluene (30 mL) was added ethyl 2-ethoxy-2-imino-acetate (237 mg, 1.63 mmol) and acetic acid (117 mg, 0.112 mL, 1.96 mmol). The mixture was stirred at 10° C. under nitrogen for 15 h. After cooling to room temperature, the mixture was diluted with ethyl acetate (20 mL), washed with water and brine. The organic layer was dried (Na₂SO₄) and concentrated in vacuo. The residue was purified by flash column chromatography (silica, 0-50% ethyl acetate in petroleum ether) to afford the title compound (120 mg, 78%) as a light yellow solid. MS m/z: 465.0 ([M+H]⁺), ESI pos.

### c) azetidin-1-yl-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

To a solution of azetidine (61.6 mg, 1.08 mmol) in tetrahydrofuran (1 mL) was added isopropylmagnesium chloride (2.0 M in tetrahydrofuran, 0.22 mL, 0.430 mmol) at 0° C. under nitrogen atmosphere. After 15 minutes, a solution of ethyl (7S)-14,14,15,15-tetradeuterio-9-(2,6-difluorophenyl)-7-methyl-13,16-dioxa-18-thia-2,3,5,8-tetrazatetracyclo[8.8.0.02,6.011,17]-octadeca-1(10),3,5,8,11(17)-pentaene-4-carboxylate (50 mg, 0.110 mmol) in tetrahydrofuran (1.0 mL) was dropped into the mixture. The mixture was stirred at 0° C. for 4 h. then diluted with dichloromethane (20 mL), washed with brine (5 mL), dried (Na₂SO₄) and concentrated in vacuo. The residue was purified by preparative TLC (silica, ethyl acetate), followed by chiral SFC (Chiralpak AD-H, 35% iPrOH) to afford the enantiopure (−)-title compound (9.3 mg, 18%) as an off-white solid. MS m/z: 476.0 ([M+H]⁺), ESI pos. ¹H NMR (CDCl₃, 400 MHz) δ ppm: 7.36 (m, 1H), 6.93 (s, 2H), 4.77-4.58 (m, 2H), 4.43-4.18 (m, 4H), 3.96 (d, J=14.4 Hz, 1H), 2.40-2.28 (m, 2H), 2.10 (d, J=6.7 Hz, 3H).

### Example 24

### (3-fluoroazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

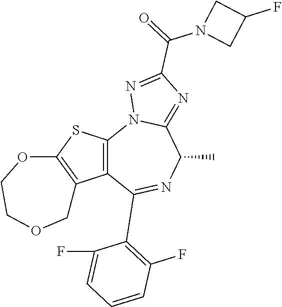 [SMILES: C[C@@H]1N=C(c2c(F)cccc2F)c2c(sc3c2COCCO3)-n2nc(C(=O)N3CC(F)C3)nc21]

The title compound was obtained in analogy to Example 22 d, using 3-fluoroazetidine instead of azetidine, as a white solid. MS m/z: 490.0 ([M+H]⁺), ESI pos.

### Example 25

### (7S)-9-(2,6-difluorophenyl)-N-(2-fluoroethyl)-7-methyl-13,16-dioxa-18-thia-2,3,5,8-tetrazatetracyclo[8.8.0.02,6.011,17]octadeca-1(10),3,5,8,11(17)-pentaene-4-carboxamide

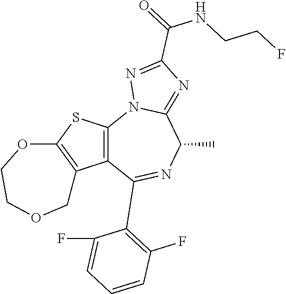 [SMILES: C[C@@H]1N=C(c2c(F)cccc2F)c2c(sc3c2COCCO3)-n2nc(C(=O)NCCF)nc21]

The title compound was obtained in analogy to Example 22 d, using 2-fluoroethylamine instead of azetidine, as a white solid. MS m/z: 478.1 ([M+H]⁺), ESI pos.

### Example 26

### (8S)-10-(2,6-difluorophenyl)-4,8-dimethyl-19-thia-2,6,9-triazatetracyclo[9.8.0.02,7.012,18]nonadeca-1(11),3,6,9,12(18)-pentaen-5-one

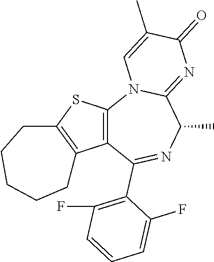 [SMILES: Cc1cn2c(nc1=O)[C@H](C)N=C(c1c(F)cccc1F)c1c-2sc2c1CCCCC2]

### a) (5S)-3-(2,6-difluorophenyl)-5-methyl-9-thia-4,7-diazatricyclo[8.5.0.02,8]pentadeca-1(10),2(8),3-trien-6-imine

A solution of (5S)-3-(2,6-difluorophenyl)-5-methyl-9-thia-4,7-diazatricyclo[8.5.0.02,8] pentadeca-1(10),2(8),3-triene-6-thione (Building block D, 112 mg, 0.297 mmol) in ammonia (7.0 M in methanol, 1.27 mL, 8.92 mmol) was stirred at 60° C. for 24 h. The reaction mixture was concentrated in vacuo. The residue was purified by flash column chromatography (silica, 0-80% ethyl acetate in heptane) to afford the title compound (99 mg, 90%) as an off-white solid. MS m/z: 360.2 ([M+H]⁺), ESI pos.

### b) (8S)-10-(2,6-difluorophenyl)-8-methyl-19-thia-2.6,9-triazatetracyclo[9.8.0.02,7.012,18]nonadeca-1(11),3.6.9.12(18)-pentaen-5-one

To a solution of (5S)-3-(2,6-difluorophenyl)-5-methyl-9-thia-4,7-diazatricyclo[8.5.0.02,8]pentadeca-1(10),2(8),3-trien-6-imine (97 mg, 0.270 mmol) in ethanol (2 mL) was added at room temperature ethyl propiolate (132 mg, 0.137 mL, 1.35 mmol). The resulting yellow solution was stirred at 60° C. for 16 h, before being concentrated in vacuo. The residue was purified by preparative HPLC (Gemini NX 5 u C18 110A, 0.05% formic acid in water/acetonitrile) to afford the title compound (31 mg, 28%) as a brown foam. MS m/z: 412.2 ([M+H]⁺), ESI pos.

### c) (8S)-4-bromo-10-(2,6-difluorophenyl)-8-methyl-19-thia-2.6,9-triazatetracyclo[9.8.0.02,7.012,18]nonadeca-1(11),3,6,9,12(18)-pentaen-5-one

To a solution of (8S)-10-(2,6-difluorophenyl)-8-methyl-19-thia-2,6,9-triazatetracyclo[9.8.0.02,7.012,18]nonadeca-1(11),3,6,9,12(18)-pentaen-5-one (209 mg, 0.508 mmol) in N,N-dimethylformamide (5 mL) was added at room temperature NBS (136 mg, 0.762 mmol). The resulting brownish solution was stirred at 70° C. for 16 h, before being concentrated in vacuo. The residue was purified by preparative HPLC (Gemini NX 5 u C18 110A, 0.05% formic acid in water/acetonitrile) to afford the title compound (195 mg, 71%) as a light yellow foam. MS m/z: 490.0 ([{⁷⁹Br}M+H]⁺), 492.1 ([{⁸¹Br}M+H]⁺), ESI pos.

### d) (8S)-10-(2,6-difluorophenyl)-4,8-dimethyl-19-thia-2.6,9-triazatetracyclo[9.8.0.02,7.012,18]nonadeca-1(11),3,6,9,12(18)-pentaen-5-one

To a solution of (8S)-4-bromo-10-(2,6-difluorophenyl)-8-methyl-19-thia-2,6,9-triazatetracyclo[9.8.0.02,7.012,18]nonadeca-1(11),3,6,9,12(18)-pentaen-5-one (59 mg, 0.120 mmol) in 1,4-dioxane (1.2 mL) and water (0.300 mL) was added trimethylboroxine (45.3 mg, 0.051 mL, 0.361 mmol) and cesium carbonate (98 mg, 0.301 mmol). The mixture was degassed with argon and 1,1′-bis(diphenylphosphino)ferrocene-palladium(II)dichloride dichloromethane complex (19.7 mg, 0.0241 mmol) was added under argon. The mixture was stirred for 16 h at 60° C., then diluted with water (5 mL) and extracted with ethyl acetate (2×20 mL). The combined organic layers were washed with brine (20 mL), dried (Na₂SO₄) and concentrated in vacuo. The residue was purified by flash column chromatography (silica, 0-80% ethyl acetate in heptane, then ethyl acetate/methanol 9:1 (v/v)), followed by chiral SFC (OD-H, 20-40% methanol) to afford the enantiopure (−)-title compound (8 mg, 16%) as a white foam. MS m/z: 426.1 ([M+H]⁺), ESI pos.

### Example 27

### (7S,14R)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-ol

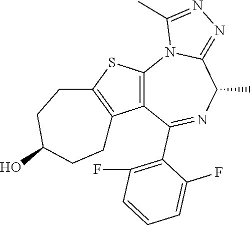 [SMILES: Cc1nnc2n1-c1sc3c(c1C(c1c(F)cccc1F)=N[C@H]2C)CC[C@@H](O)CC3]

To a solution of (7S)-9-(2,6-difluorophenyl)-3,7-dimethyl-18-thia-2,4,5,8-tetrazatetracyclo[8.8.0.02,6.011,17] octadeca-1(10),3,5,8,11(17)-pentaen-14-one (Building block H, 1.60 g, 3.88 mmol) in methanol (40 mL) was added at 0-4° C. (ice bath) lithium borohydride (253 mg, 11.6 mmol). The reaction mixture was stirred at room temperature for 3 h, before being quenched by addition of ice-water (20 mL). The mixture was partitioned between dichloromethane (50 mL) and semi-saturated aqueous ammonium chloride (20 mL). The aqueous phase was extracted with dichloromethane (2×50 mL). The combined organic layers were washed with water (50 mL) and brine (50 mL), dried (Na₂SO₄) and concentrated in vacuo. A partial amount (460 mg) of the resulting diastereomeric mixture was separated by chiral SFC (column chiral IH, 20-40% methanol) to afford the enantiopure (−)-title compound (119 mg, 26%) as an off-white foam. MS: m/z: 415.1 ([M+H]⁺), ESI pos.

### Example 28

### (7S)-9-(2,6-difluorophenyl)-4,7-dimethyl-13,16-dioxa-18-thia-2,3,5,8-tetrazatetracyclo[8.8.0.02,6.011,17]octadeca-1(10),3,5,8,11(17)-pentaene

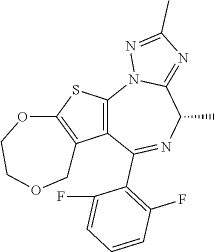 [SMILES: Cc1nc2n(n1)-c1sc3c(c1C(c1c(F)cccc1F)=N[C@H]2C)COCCO3]

### a) (13S)-11-amino-15-(2,6-difluorophenyl)-13-methyl-4,7-dioxa-9-thia-11,14-diazatricyclo[8.5.0.02,8]pentadeca-1(10),2(8),14-trien-12-one

To a solution of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K, 1.5 g, 4.3 mmol) and O-(diphenylphosphinyl)hydroxylamine (1.2 g, 5.1 mmol) in N,N-dimethylformamide (20 mL) was added cesium carbonate (2.1 g, 6.4 mmol) at 0° C. The reaction mixture was stirred at room temperature for 4 h, before being diluted with ethyl acetate (100 mL). The organic layer was washed with water (3×50 mL), dried (Na₂SO₄) and concentrated in vacuo. The residue was purified by flash column chromatography (silica, petroleum ether/ethyl acetate, 5:1 to 1:1) to afford the title compound (770 mg, 52%) as a light yellow solid. MS m/z: 380.0 ([M+H]⁺), ESI pos.

### b) (7S)-9-(2,6-difluorophenyl)-4,7-dimethyl-13,16-dioxa-18-thia-2.3.5,8-tetrazatetracyclo-[8.8.0.02,6.011,17]octadeca-1(101.3.5.8.11(17)-pentaene

A mixture of (13S)-11-amino-15-(2,6-difluorophenyl)-13-methyl-4,7-dioxa-9-thia-11,14-diazatricyclo[8.5.0.02,8]pentadeca-1(10),2(8),14-trien-12-one (144 mg, 0.380 mmol), thioacetamide (750 mg, 9.49 mmol) and zinc chloride (103 mg, 0.759 mmol) was heated to 120° C. for 12 h to form a brown liquid. The mixture was allowed to cool to room temperature and the crude product was purified by flash column chromatography (silica, 5-50% ethyl acetate in heptane), followed by chiral SFC (chiral IG, 15% methanol) to afford the enantiopure (−)-title compound (20 mg, 13%) as a white solid. MS m/z: 403.3 ([M+H]⁺), ESI pos.

### Example 29

### (3-tert-butoxyazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

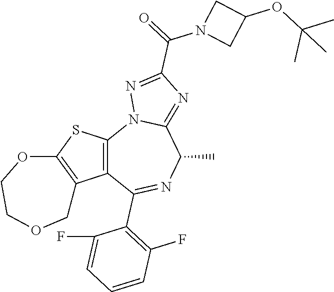 [SMILES: C[C@@H]1N=C(c2c(F)cccc2F)c2c(sc3c2COCCO3)-n2nc(C(=O)N3CC(OC(C)(C)C)C3)nc21]

The title compound was obtained in analogy to Example 22 d, using 3-tert-butoxyazetidine instead of azetidine, as a white solid. MS m/z: 544.1 ([M+H]⁺), ESI pos.

### Example 30

### (7S,15S)-9-(2,6-difluorophenyl)-3,7,15-trimethyl-13,16-dioxa-18-thia-2,4,5,8-tetrazatetracyclo[8.8.0.02,6.011,17]octadeca-1(10),3,5,8,11(17)-pentaene

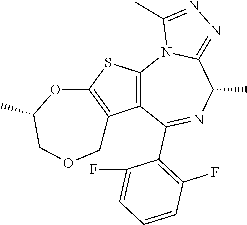

To a stirred solution of (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N, 100 mg, 0.3 mmol) in anhydrous tetrahydrofuran (10 mL) was added sodium hydride (60% dispersion in mineral oil, 21 mg, 0.5 mmol) at 0° C. After 10 min, bis(2-oxo-3-oxazolidinyl)phosphinic chloride (134.5 mg, 0.5 mmol) was added and the solution was stirred at 0° C. for 2 h, before addition of acethydrazide (97.8 mg, 1.3 mmol). The solution was stirred at room temperature for 3 h, then heated to 60° C. for 10 h. Finally, 4 Å Molecular sieves (500 mg) and silica gel (500 mg) were added, and the mixture was stirred at 60° C. for 12 h. The mixture was allowed to cool to room temperature, before being quenched by addition of saturated aqueous ammonium chloride. The mixture was extracted twice with ethyl acetate. The combined organic layers were dried (MgSO₄) then concentrated in vacuo. The residue was purified by flash column chromatography (silica, 0-80% ethyl acetate in petroleum ether), followed by chiral SFC (Daicel CHIRALPAK® IC, 10-40% methanol) to afford the enantiopure (−)-title compound (9 mg, 8%) as a off-white solid. MS m/z: 416.7 ([M+H]⁺), ESI pos.

### Example 31

### [(7S)-9-(2,6-Difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]-(3-ethoxyazetidin-1-yl)methanone

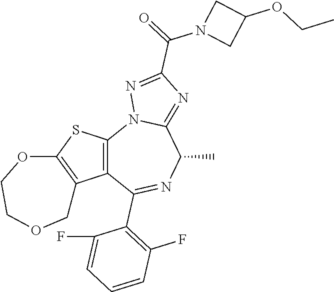 [SMILES: CCOC1CN(C(=O)c2nc3n(n2)-c2sc4c(c2C(c2c(F)cccc2F)=N[C@H]3C)COCCO4)C1]

The title compound was obtained in analogy to Example 22 d, using 3-ethoxyazetidine instead of azetidine, as an off-white solid. MS m/z: 515.7 ([M+H]⁺), ESI pos.

### Example 32

### (3-Ethoxyazetidin-1-yl)-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

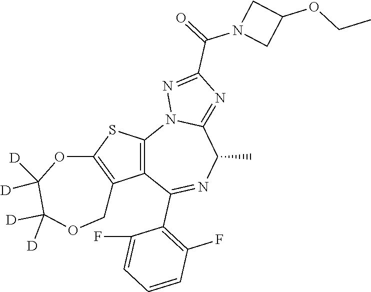 [SMILES: [2H]C1([2H])OCc2c(sc3c2C(c2c(F)cccc2F)=N[C@@H](C)c2nc(C(=O)N4CC(OCC)C4)nn2-3)OC1([2H])[2H]]

### a) (13S)-11-amino-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one

To a solution of (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (Building block M, 202 mg, 0.550 mmol) and O-(diphenylphosphinyl)hydroxylamine (154 mg, 0.660 mmol) in N,N-dimethylformamide (10 mL) was added cesium carbonate (268.26 mg, 0.820 mmol) at 0° C. The mixture was stirred at room temperature for 4 h, before being diluted with ethyl acetate (50 mL). The organic phase was washed with water (3×20 mL), dried (Na₂SO₄) and concentrated in vacuo. The residue was purified by flash column chromatography (silica, 0-100% ethyl acetate in petroleum ether) to afford the title compound (125 mg, 54%) as a light yellow solid. MS m/z: 383.7 ([M+H]⁺), ESI pos.

### b) ethyl (7S)-14,14,15,15-tetradeuterio-9-(2,6-difluorophenyl)-7-methyl-13,16-dioxa-18-thia-2,3,5,8-tetrazatetracyclo[8.8.0.02,6.011,17]-octadeca-1(10),3,5,8,11(17)-pentaene-4-carboxylate

To a solution of (13S)-11-amino-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (125 mg, 0.33 mmol) in toluene (30 mL) was added ethyl 2-ethoxy-2-imino-acetate (237 mg, 1.63 mmol) and acetic acid (117.47 mg, 1.96 mmol). The mixture was stirred at 100° C. under nitrogen for 15 h. After cooling to room temperature, the mixture was diluted with ethyl acetate (20 mL) and the organic phase washed with water and brine. The organic layer was dried (Na₂SO₄) and concentrated in vacuo. The residue was purified by flash column chromatography (silica, 0-50% ethyl acetate in petroleum ether) to afford the title compound (120 mg, 78%) as light yellow solid. MS m/z: 465.0 ([M+H]⁺), ESI pos.

### c) (7S)-14,14,15,15-tetradeuterio-9-(2,6-difluorophenyl)-7-methyl-13,16-dioxa-18-thia-2,3,5,8-tetrazatetracyclo [8.8.0.02,6.011,17] octadeca-1(10),3,5,8,11(17-pentaene-4-carboxylic acid

To a solution of ethyl (7S)-14,14,15,15-tetradeuterio-9-(2,6-difluorophenyl)-7-methyl-13,16-dioxa-18-thia-2,3,5,8-tetrazatetracyclo[8.8.0.02,6.011,17]-octadeca-1(10),3,5,8,11(17)-pentaene-4-carboxylate (120 mg, 0.258 mmol) in methanol (1 mL) was added aqueous sodium hydroxide (2.0 M, 0.22 mL, 0.430 mmol). The mixture was stirred at room temperature for 1 h, before addition of aqueous hydrochloric acid (1.0 M) to pH=5. The mixture was diluted with dichloromethane (30 mL). The organic layer was washed with brine, dried (Na₂SO₄) and concentrated in vacuo to afford the title compound (90 mg, 75%) as light yellow solid, which was used without further purification. MS m/z: 436.7 ([M+H]⁺), ESI pos.

### d) (3-ethoxyazetidin-1-yl)-[(7S)-14.14.15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone

To a solution of (7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylic acid (90 mg, 0.206 mmol) and DIPEA (0.11 mL, 0.618 mmol) in dichloromethane (10 mL) was added propylphosphonic anhydride (269 mg, 0.412 mmol) at room temperature. The resulting mixture was stirred at room temperature for 3 h, then diluted with dichloromethane (20 mL). The organic layer was washed with brine (5 mL), dried (Na₂SO₄) and concentrated in vacuo. The residue was purified by preparative TLC (silica, ethyl acetate), followed by chiral SFC (Chiralpak AD-H, 35% methanol) to afford the enantiopure (−)-title compound (30.2 mg, 28%) as a white solid. MS m/z: 520.0 ([M+H]⁺), ESI pos.

### Example 33

### (7S)-9-(2,6-difluorophenyl)-3,7-dimethyl-12-oxa-18-thia-2,4,5,8-tetrazatetracyclo[8.8.0.02,6.011,17]octadeca-1(10),3,5,8,11(17)-pentaene

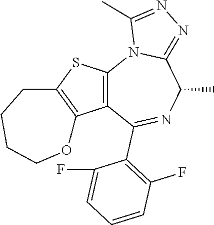 [SMILES: Cc1nnc2n1-c1sc3c(c1C(c1c(F)cccc1F)=N[C@H]2C)OCCCC3]

To a solution of (13S)-15-(2,6-difluorophenyl)-13-methyl-3-oxa-9-thia-11,14-diazatricyclo[8.5.0.02,8]pentadeca-1(10),2(8),14-triene-12-thione (Building block O, 31 mg, 0.080 mmol) in n-butanol (0.8 mL) was added acethydrazide (31.3 mg, 0.401 mmol). The reaction mixture was stirred in the microwave at 130° C. for 2 h, before being concentrated in vacuo. The residue was purified by flash column chromatography (silica, 0-5% methanol in dichloromethane), followed by chiral SFC (chiral IA, 15% methanol) to afford the enantiopure (−)-title compound (8 mg, 24%) as a light yellow solid. MS m/z: 401.4 ([M+H]⁺), ESI pos.

### Example 34

### (7S)-9-(2,6-difluorophenyl)-14,15-difluoro-3,7-dimethyl-18-thia-2,4,5,8-tetrazatetracyclo[8.8.0.02,6.011,17]octadeca-1(10),3,5,8,11(17)-pentaene

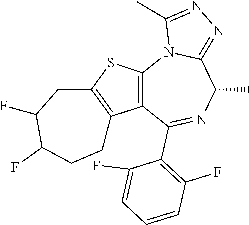 [SMILES: Cc1nnc2n1-c1sc3c(c1C(c1c(F)cccc1F)=N[C@H]2C)CCC(F)C(F)C3]

### a) (7S)-9-(2,6-difluorophenyl)-3,7-dimethyl-18-thia-2,4,5,8-tetrazatetracyclo[8.8.0.02,6.011,17]octadeca-1(10),3,5,8,11(17),14-hexaene

To a solution of (5S)-3-(2,6-difluorophenyl)-5-methyl-9-thia-4,7-diazatricyclo[8.5.0.02,8]pentadeca-1(10),2(8),3,12-tetraen-6-one (Building block Q, 9.7 g, 27.06 mmol) in anhydrous tetrahydrofuran (400 mL) was added sodium hydride (60% dispersion in mineral oil, 2.17 g, 54.13 mmol) at 0° C. After 10 min, bis(2-oxo-3-oxazolidinyl)phosphinic chloride (13.78 g, 54.1 mmol) was added and the solution was stirred at 0° C. for 2 h, before addition of acethydrazide (10.0 g, 135.3 mmol). The reaction mixture was stirred at room temperature for 3 h, then heated to 60° C. for 16 h. After cooling to room temperature, the mixture was quenched by addition of saturated aqueous ammonium chloride. The mixture was extracted with ethyl acetate (2×250 mL) and the combined organic layers were dried (MgSO₄) and concentrated in vacuo. The residue was purified by flash column chromatography (silica, 0-80% ethyl acetate in petroleum ether), followed by SFC (Chiralpak IC, 35% ethanol and 0.2% ammonium hydroxide) to afford the title compound (3.8 g, 35%) as a white solid. MS m/z: 396.9 ([M+H]⁺), ESI pos.

### b) (7S)-9-(2,6-difluorophenyl)-14,15-difluoro-3,7-dimethyl-18-thia-2,4,5,8-tetrazatetracyclo[8.8.0.02,6.011,17]octadeca-1(10),3,5,8,11(17)-pentaene

Into a polyethylene flask containing Olah's Reagent (285 mg, 2.02 mmol) and dichloromethane (15 mL) was added N-bromosuccinimide (90 mg, 0.50 mmol). The vessel was cooled to 0° C. and (7S)-9-(2,6-difluorophenyl)-3,7-dimethyl-18-thia-2,4,5,8-tetrazatetracyclo-[8.8.0.02,6.011,17]octadeca-1(10),3,5,8,11(17),14-hexaene (200 mg, 0.50 mmol) was added. The reaction mixture was allowed to warm to room temperature and stirred for 60 min. Thereafter, silver(I)-fluoride (192 mg, 1.51 mmol) was added and the reaction was stirred for further 12 h at room temperature. The reaction mixture was poured into ice-water and extracted with ethyl acetate (2×50 mL). The combined organic layers were washed with water, aqueous potassium hydroxide and water, dried (Na₂SO₄) and concentrated in vacuo. The residue was purified by preparative HPLC (Gemini C18, acetonitrile/water 5-65%), followed by chiral SFC (Chiralpak IC, 35% ethanol and 0.2% ammonium hydroxide) to afford the enantiopure (−)-title compound (4.7 mg, 2%) as an off-white solid. MS m/z: 434.7 ([M+H]⁺), ESI pos.

### Example 35

### (7S)-9-(2-chloro-6-fluoro-phenyl)-3,7-dimethyl-13,16-dioxa-18-thia-2,4,5,8-tetrazatetracyclo[8.8.0.02,6.011,17]octadeca-1(10),3,5,8,11(17)-pentaene

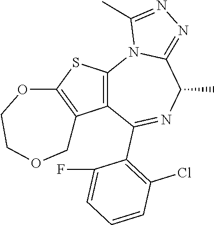 [SMILES: Cc1nnc2n1-c1sc3c(c1C(c1c(F)cccc1Cl)=N[C@H]2C)COCCO3]

The title compound was obtained in analogy to Example 21, using (13S)-15-(2-chloro-6-fluoro-phenyl)-13-methyl-4,7-dioxa-9-thia-11,14-diazatricyclo[8.5.0.02,8]pentadeca-1(10),2(8),14-triene-12-thione (Building block P) instead of (13S)-15-(2,6-difluorophenyl)-13-methyl-4,7-dioxa-9-thia-11,14-diazatricyclo[8.5.0.02,8] pentadeca-1(10),2(8),14-triene-12-thione (Building block L), as a light yellow solid. MS m/z: 419.2 ([{³⁵Cl}M+H]⁺), 421.2 ([{³⁷Cl}M+H]⁺), ESI pos.

### Example 36

### [(7S)—N-cyclopropyl-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxamide

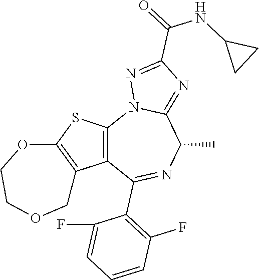 [SMILES: C[C@@H]1N=C(c2c(F)cccc2F)c2c(sc3c2COCCO3)-n2nc(C(=O)NC3CC3)nc21]

The title compound was obtained in analogy to Example 23, using (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K) instead of (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (Building block M) in step a) and cyclopropylamine instead of azetidine in step c), as a white solid. MS m/z: 472.0 ([M+H]⁺), ESI pos.

### Example 37

### (7S)-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide

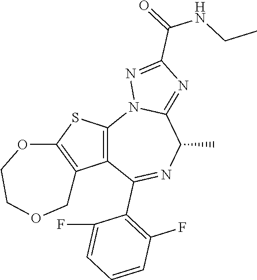 [SMILES: CCNC(=O)c1nc2n(n1)-c1sc3c(c1C(c1c(F)cccc1F)=N[C@H]2C)COCCO3]

The title compound was obtained in analogy to Example 23, using (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K) instead of (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (Building block M) in step a) and ethylamine instead of azetidine in step c), as a white solid. MS m/z: 460.0 ([M+H]⁺), ESI pos.

### Example 38

### (7S)-14,14,15,15-tetradeuterio-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide

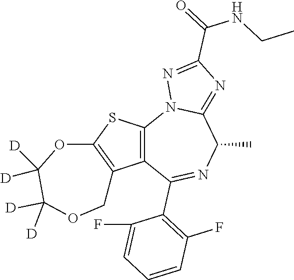 [SMILES: [2H]C1([2H])OCc2c(sc3c2C(c2c(F)cccc2F)=N[C@@H](C)c2nc(C(=O)NCC)nn2-3)OC1([2H])[2H]]

The title compound was obtained in analogy to Example 23 c, using ethylamine instead of azetidine, as a white solid. MS m/z: 464.0 ([M+H]⁺), ESI pos.

### Example 39

### (7S,15S)-9-(2,6-difluorophenyl)-4,7,15-trimethyl-13,16-dioxa-18-thia-2,3,5,8-tetrazatetracyclo[8.8.0.02,6.011,17]octadeca-1(10),3,5,8,11(17)-pentaene

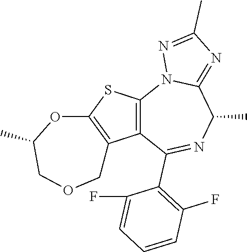

### a) (6S,13S)-11-amino-15-(2,6-difluorophenyl)-6,13-dimethyl-4,7-dioxa-9-thia-11,14-diazatricyclo[8.5.0.02,8]pentadeca-1(10),2(8),14-trien-12-one

The title compound was obtained in analogy to Example 28 a, using (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N) instead of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K), as a white solid, which was used directly in the next step.

### b) (7S,15S)-9-(2,6-difluorophenyl)-4,7,15-trimethyl-13,16-dioxa-18-thia-2,3,5,8-tetrazatetracyclo[8.8.0.02,6.011,17]octadeca-1(10),3,5,8,11(17)-pentaene

To a solution of (6S,13S)-11-amino-15-(2,6-difluorophenyl)-6,13-dimethyl-4,7-dioxa-9-thia-11,14-diazatricyclo[8.5.0.02,8]pentadeca-1(10),2(8),14-trien-12-one (50 mg, 0.130 mmol) in pyridine (1 mL) was added ethyl acetimidate hydrochloride (220 mg, 1.78 mmol). The solution was stirred at 100° C. for 12 h, then concentrated in vacuo. The residue was purified by chiral SFC (Chiralpak AD-H, 35% iPrOH and 0.2% ammonium hydroxide) to afford the enantiopure (−)-title compound (5.5 mg, 10%) as a white solid. MS m/z: 416.7 ([M+H]⁺), ESI pos.

### Example 40

### [(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]-(3-fluoroazetidin-1-yl)methanone

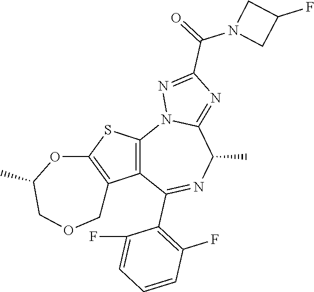 [SMILES: C[C@@H]1N=C(c2c(F)cccc2F)c2c(sc3c2COC[C@H](C)O3)-n2nc(C(=O)N3CC(F)C3)nc21]

The title compound was obtained in analogy to Example 22, using (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N) instead of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K) in step a) and 3-fluoroazetidine hydrochloride instead of azetidine in step d), as a white solid. MS m/z: 503.7 ([M+H]⁺), ESI pos.

### Example 41

### (3-tert-butoxyazetidin-1-yl)-[(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]methanone

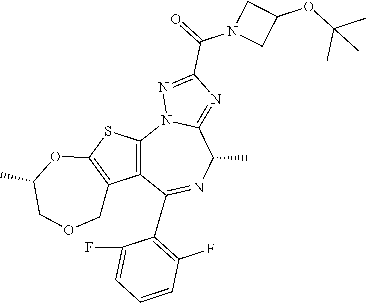 [SMILES: C[C@@H]1N=C(c2c(F)cccc2F)c2c(sc3c2COC[C@H](C)O3)-n2nc(C(=O)N3CC(OC(C)(C)C)C3)nc21]

The title compound was obtained in analogy to Example 22, using (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N) instead of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K) in step a) and 3-tert-butoxyazetidine instead of azetidine in step d), as a white solid. MS m/z: 557.8 ([M+H]⁺), ESI pos.

### Example 42

### (7S)-9-(2,6-difluorophenyl)-3-ethyl-7-methyl-13,16-dioxa-18-thia-2,4,5,8-tetrazatetracyclo[8.8.0.02,6.011,17]octadeca-1(10),3,5,8,11(17)-pentaene

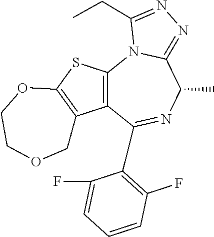 [SMILES: CCc1nnc2n1-c1sc3c(c1C(c1c(F)cccc1F)=N[C@H]2C)COCCO3]

To a stirred solution of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K, 46.2 mg, 0.130 mmol) in anhydrous tetrahydrofuran (3 mL) was added sodium hydride (60% dispersion in mineral oil, 10.1 mg, 0.250 mmol) at 0° C. After 10 min, bis(2-oxo-3-oxazolidinyl)phosphinic chloride (64.5 mg, 0.250 mmol) was added (within 30 min) and the solution was stirred at 0° C. for further 2 h. Finally, propanoic acid hydrazide (55.8 mg, 0.630 mmol) was added and the mixture was heated to 60° C. for 12 h. The reaction mixture was diluted with ethyl acetate (15 mL). The organic phase was washed with brine (15 mL), dried (Na₂SO₄) and concentrated in vacuo. The residue was purified by preparative HPLC (Gemini 5 u C18, acetonitrile/water), followed by chiral SFC (Chiralpak AD-H, 35% iPrOH) to afford the enantiopure (−)-title compound (18.1 mg, 34%) as a white solid. MS m/z: 416.7 ([M+H]⁺), ESI pos.

### Example 43

### (7S)-3-cyclopropyl-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene

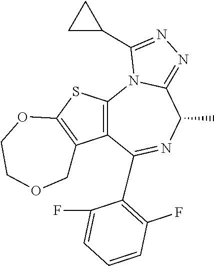 [SMILES: C[C@@H]1N=C(c2c(F)cccc2F)c2c(sc3c2COCCO3)-n2c(C3CC3)nnc21]

The title compound was obtained in analogy to Example 42, using cyclopropane carbohydrazide instead of propanoic acid hydrazide, as white solid. MS m/z: 428.7 ([M+H]⁺), ESI pos.

### Example 44

### (7S)-9-(2,6-difluorophenyl)-3-isopropyl-7-methyl-13,16-dioxa-18-thia-2,4,5,8-tetrazatetracyclo[8.8.0.02,6.011,17]octadeca-1(10),3,5,8,11(17)-pentaene

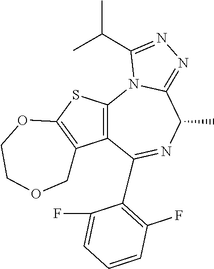 [SMILES: CC(C)c1nnc2n1-c1sc3c(c1C(c1c(F)cccc1F)=N[C@H]2C)COCCO3]

The title compound was obtained in analogy to Example 42, using isobutyric acid hydrazide instead of propanoic acid hydrazide, as white solid. MS m/z: 430.7 ([M+H]⁺), ESI pos.

### Example 45

### (7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide

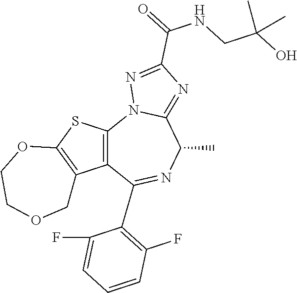 [SMILES: C[C@@H]1N=C(c2c(F)cccc2F)c2c(sc3c2COCCO3)-n2nc(C(=O)NCC(C)(C)O)nc21]

The title compound was obtained in analogy to Example 23, using (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K) instead of (13S)-5,5,6,6-tetradeuterio-15-(2,6-difluorophenyl)-13-methyl-4,7-dioxa-9-thia-11,14-diazatricyclo[8.5.0.02,8]pentadeca-1(10),2(8),14-trien-12-one (Building block M) in step a) and 1-amino-2-methylpropan-2-ol instead of azetidine in step c), as a white solid. MS m/z: 504.0 ([M+H]⁺), ESI pos.

### Example 46

### (7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide

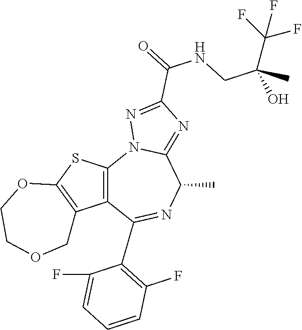 [SMILES: C[C@@H]1N=C(c2c(F)cccc2F)c2c(sc3c2COCCO3)-n2nc(C(=O)NC[C@@](C)(O)C(F)(F)F)nc21]

The title compound was obtained together with (7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide in analogy to Example 22 d, using 3-amino-1,1,1-trifluoro-2-methyl-propan-2-ol hydrochloride instead of azetidine. The diastereomers were separated by chiral SFC (CHIRALPAK WK-01, 35% methanol and 0.2% ammonium hydroxide) to afford the title compound as a white solid. MS m/z: 558.0 ([M+H]⁺), ESI pos. ¹H NMR (CDCl₃, 400 MHz) δ ppm: 7.62 (t, J=6.4 Hz, 1H), 7.39 (m, 1H), 6.94 (s, 2H), 4.37 (d, J=6.5 Hz, 1H), 4.29-4.14 (m, 3H), 4.06-3.92 (m, 2H), 3.87 (m, 1H), 3.83-3.71 (m, 2H), 2.10 (d, J=6.8 Hz, 3H), 1.41 (s, 3H).

### Example 47

### (7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide

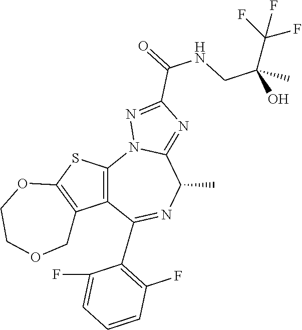 [SMILES: C[C@@H]1N=C(c2c(F)cccc2F)c2c(sc3c2COCCO3)-n2nc(C(=O)NC[C@](C)(O)C(F)(F)F)nc21]

The title compound was obtained together with (7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide in analogy to Example 22 d, using 3-amino-1,1,1-trifluoro-2-methyl-propan-2-ol hydrochloride instead of azetidine. The diastereomers were separated by chiral SFC (CHIRALPAK WK-01, 35% methanol and 0.2% ammonium hydroxide) to afford the title compound as a white solid. MS m/z: 558.0 ([M+H]⁺), ESI pos. ¹H NMR (CDCl₃, 400 MHz) δ ppm: 7.64 (t, J=6.3 Hz, 1H), 7.38 (m, 1H), 6.94 (s, 2H), 4.36 (d, J=6.6 Hz, 1H), 4.30-4.12 (m, 3H), 4.04-3.92 (m, 2H), 3.92-3.81 (m, 1H), 3.75 (m, 2H), 2.09 (d, J=6.7 Hz, 3H), 1.40 (s, 3H).

NOTE: The configuration at the chiral tertiary alcohol was determined by comparison of retention times with a sample prepared from (2S)-3-amino-1,1,1-trifluoro-2-methyl-propan-2-ol instead of the racemic 3-amino-1,1,1-trifluoro-2-methyl-propan-2-ol.

### Example 48

### (7S)-3-(difluoromethyl)-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene

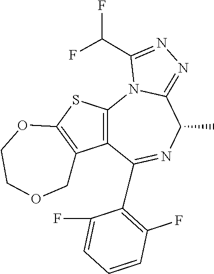 [SMILES: C[C@@H]1N=C(c2c(F)cccc2F)c2c(sc3c2COCCO3)-n2c(C(F)F)nnc21]

The title compound was obtained in analogy to Example 42, using 2,2-difluoroacetohydrazide instead of propanoic acid hydrazide, as brown powder. MS m/z: 438.7 ([M+H]⁺), ESI pos.

### Example 49

### (7S,15S)-9-(2,6-difluorophenyl)-4-ethyl-7,15-dimethyl-13,16-dioxa-18-thia-2,3,5,8-tetrazatetracyclo[8.8.0.02,6.011,17]octadeca-1(10),3,5,8,11(17)-pentaene

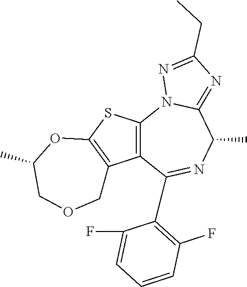

The title compound was obtained in analogy to Example 39 b, using ethyl propanimidate hydrochloride instead of ethyl acetimidate hydrochloride, as a light red solid. MS m/z: 430.7 ([M+H]⁺), ESI pos.

### Example 50

### (7S)-9-(2,6-difluorophenyl)-3,7-dimethyl-13,16-dioxa-18-thia-2,5,8-triazatetracyclo[8.8.0.02,6.011,17]octadeca-1(10),3,5,8,11(17)-pentaene

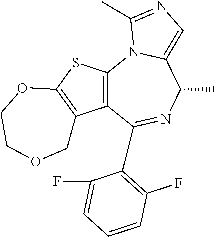 [SMILES: Cc1ncc2n1-c1sc3c(c1C(c1c(F)cccc1F)=N[C@H]2C)COCCO3]

### a) (13S)-15-(2,6-difluorophenyl)-13-methyl-4,7-dioxa-9-thia-11,14-diazatricyclo[8.5.0.02,8]pentadeca-1(10),2(8),14-trien-12-imine

A solution of ammonia (7.0 M in methanol, 3.19 mL, 22.34 mmol) and (13S)-15-(2,6-difluorophenyl)-13-methyl-4,7-dioxa-9-thia-11,14-diazatricyclo[8.5.0.02,8] pentadeca-1(10),2(8),14-triene-12-thione (Building block L, 170 mg, 0.45 mmol) in methanol (3.2 mL) in a seal-tube under a nitrogen atmosphere was heated to 60° C. for 5 h. The reaction mixture was concentrated in vacuo. The residue was purified by flash column chromatography (silica, ethyl acetate in petroleum ether) to afford the title compound (115 mg, 71%) as a yellow powder. MS m/z: 364.1 ([M+H]⁺), ESI pos.

### b) (7S)-9-(2,6-difluorophenyl)-3,7-dimethyl-13,16-dioxa-18-thia-2,5,8-triazatetracyclo[8.8.0.02,6.011,17]octadeca-1(10),3,5,8,11(17)-pentaene

To a solution of (13S)-15-(2,6-difluorophenyl)-13-methyl-4,7-dioxa-9-thia-11,14-diazatricyclo[8.5.0.02,8]pentadeca-1(10),2(8),14-trien-12-imine (115 mg, 0.320 mmol) in 1-butanol (10 mL) under a nitrogen atmosphere was added propargylamine (87.2 mg, 1.58 mmol) and p-toluenesulfonic acid (5.5 mg, 0.030 mmol). The reaction mixture was stirred in a microwave reactor at 150° C. for 5 h, before being concentrated in vacuo. The residue was purified by flash column chromatography (silica, dichloromethane/methanol 97:3), followed by preparative HPLC (Gemini-C18, acetonitrile/water (0.1% formic acid) 20-50%) and, finally, by chiral SFC (chiralpak-IC, EtOH) to afford the enantiopure (−)-title compound (23.7 mg, 19%) as a light yellow amorphous freeze-dried solid. MS m/z: 402.2 ([M+H]⁺), ESI pos.

### Example 51

### (7S,14R)-9-(2,6-difluorophenyl)-3,7-dimethyl-18-thia-2,5,8-triazatetracyclo[8.8.0.02,6.011,17]octadeca-1(10),3,5,8,11(17)-pentaen-14-ol

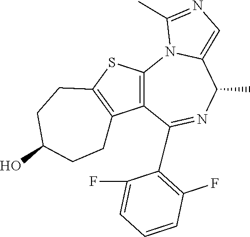 [SMILES: Cc1ncc2n1-c1sc3c(c1C(c1c(F)cccc1F)=N[C@H]2C)CC[C@@H](O)CC3]

### a) (5'S)-3′-(2,6-difluorophenyl)-5′-methyl-spiro[1,3-dioxolane-2,12′-9-thia-4,7-diazatricyclo[8.5.0.02,8]pentadeca-1(10),2(8),3-triene]-6′-thione

A solution of (5'S)-3′-(2,6-difluorophenyl)-5′-methyl-spiro[1,3-dioxolane-2,13′-9-thia-4,7-diazatricyclo[8.5.0.02,8]pentadeca-1(10),2(8),3-triene]-6′-one (Building block G d, 13.0 g, 31.1 mmol) and pyridine (7.54 mL, 93.2 mmol) in dichloromethane (500 mL) was cooled to −40° C., before addition of trifluoromethanesulfonic acid (5.59 g, 37.2 mmol). The mixture was allowed gradually to warm to −5° C. over a period of 2 h, then was stirred for an additional 2 h at room temperature. The reaction mixture was re-cooled to −5° C., before being quenched by addition of aqueous ammonium sulfide (15.87 g, 46.6 mmol). After 2 h at −5° C., the crude reaction was purified by flash column chromatography (silica, 0-80% ethyl acetate in petroleum ether) to afford the title compound (8 g, 53%) as a yellow solid. MS m/z: 434.7 ([M+H]⁺), ESI pos.

### b) (5'S)-3′-(2,6-difluorophenyl)-5′-methyl-spiro[1,3-dioxolane-2,13′-9-thia-4,7-diazatricyclo[8.5.0.02,8]pentadeca-1(10),2(8),3-triene]-6′-imine

To a solution of (5'S)-3′-(2,6-difluorophenyl)-5′-methyl-spiro[1,3-dioxolane-2,12′-9-thia-4,7-diazatricyclo[8.5.0.02,8]pentadeca-1(10),2(8),3-triene]-6′-thione (8.0 g, 18.4 mmol) in a seal tube was added ammonia (7.0 M in methanol, 80 mL). The tube was sealed and the reaction mixture was heated at 60° C. for 12 h, before being concentrated in vacuo. The residue was purified by flash column chromatography (silica, 0-100% ethyl acetate in hexanes) to afford the title compound (5.6 g, 74%) as a yellow solid. MS m/z: 418.1 ([M+H]⁺), ESI pos.

### c (7′S)-9′-(2,6-difluorophenyl)-3′,7′-dimethyl-spiro[1,3-dioxolane-2,14′-18-thia-2,5,8-triazatetracyclo[8.8.0.02,6.011,17]octadeca-1(10),3,5,8,11(17)-pentaene]

A mixture of (5'S)-3′-(2,6-difluorophenyl)-5′-methyl-spiro[1,3-dioxolane-2,13′-9-thia-4,7-diazatricyclo[8.5.0.02,8] pentadeca-1(10),2(8),3-triene]-6′-imine (1.5 g, 3.59 mmol), propargylamine (1.58 g, 28.7 mmol), p-toluenesulfonamide (68.27 mg, 0.360 mmol) was heated at reflux overnight. The reaction mixture was concentrated in vacuo and the residue was taken up in dichloromethane (100 mL). The organic phase was washed with saturated aqueous sodium hydrogen carbonate, dried (Na₂SO₄) and concentrated in vacuo. The residue was purified by flash column chromatography (silica, 0-5% methanol in dichloromethane) to afford the title compound (1.1 g, 64%) as a light brown solid. MS m/z: 455.7 ([M+H]⁺), ESI pos.

### d) (7S)-9-(2,6-difluorophenyl)-3,7-dimethyl-18-thia-2.5,8-triazatetracyclo[8.8.0.02,6.011,17]octadeca-1(10),3,5,8,11(17)-pentaen-14-one

In a microwave vial (7'S)-9′-(2,6-difluorophenyl)-3′,7′-dimethyl-spiro[1,3-dioxolane-2,14′-18-thia-2,5,8-triazatetracyclo[8.8.0.02,6.011,17]octadeca-1(10),3,5,8,11(17)-pentaene] (472 mg, 1.0 mmol) was dissolved in a mixture of acetic acid (4 mL), water (5 mL) and acetone (5 mL). The reaction mixture was stirred under microwave irradiation at 120° C. for 60 min, before being poured into ice water and basified by careful addition of solid sodium hydrogen carbonate. The aqueous layer was extracted with ethyl acetate (2×300 mL). The combined organic layers were washed with brine (250 mL), dried (Na₂SO₄) and concentrated in vacuo to afford the title compound (350 mg, 73%) as a light yellow oil. MS m/z: 411.8 ([M+H]⁺), ESI pos.

### e) (7S,14R)-9-(2,6-difluorophenyl)-3,7-dimethyl-18-thia-2.5,8-triazatetracyclo[8.8.0.02,6.011,17]octadeca-1(10),3,5,8,11(17)-pentaen-14-ol

To a solution of (7S)-9-(2,6-difluorophenyl)-3,7-dimethyl-18-thia-2,5,8-triazatetracyclo[8.8.0.02,6.011,17]octadeca-1(10),3,5,8,11(17)-pentaen-14-one (800 mg, 1.94 mmol) in methanol (20 mL) was added lithium borohydride (128 mg, 5.82 mmol) at 0° C. The reaction mixture was allowed to warm to room temperature and stirred for 3 h. The reaction was quenched by addition of ice-water and extracted with dichloromethane. The organic phase was washed with saturated aqueous ammonium chloride, dried (Na₂SO₄) and concentrated in vacuo. The residue was purified by flash column chromatography (silica, 0-5% methanol in dichloromethane), followed by chiral SFC (chiral cellulose-4, 40% methanol, second eluting diastereomer) to afford the title compound (80 mg, 10%) as a white solid. MS m/z: 414.2 ([M+H]⁺), ESI pos. ¹H NMR (CDCl₃, 300 MHz) δ ppm: 7.34-7.22 (m, 2H), 6.89 (br d, J=1.0 Hz, 3H), 4.17 (q, J=6.6 Hz, 1H), 3.94 (br t, J=7.8 Hz, 1H), 3.48 (s, 1H), 3.04 (ddd, J=1.8, 9.5, 15.9 Hz, 1H), 2.68-2.55 (m, 2H), 2.43 (d, J=1.0 Hz, 3H), 2.07-1.91 (m, 5H), 1.84-1.69 (m, 2H), 1.64-1.62 (m, 1H), 1.52-1.33 (m, 1H), 1.32-1.05 (m, 1H).

### Example 52

### (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaene

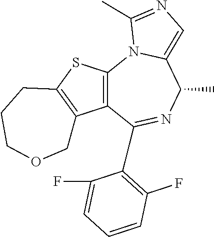 [SMILES: Cc1ncc2n1-c1sc3c(c1C(c1c(F)cccc1F)=N[C@H]2C)COCCC3]

To a stirred solution of (5S)-3-(2,6-difluorophenyl)-5-methyl-14-oxa-9-thia-4,7-diazatricyclo[8.5.0.02,8]pentadeca-1(10),2(8),3-trien-6-one (Building block R, 120 mg, 0.330 mmol) in anhydrous tetrahydrofuran (5 mL) was added sodium hydride (60% dispersion in mineral oil, 26.5 mg, 0.660 mmol) at 0° C. under a nitrogen atmosphere. The resulting suspension was stirred at 0° C. for 30 min, before addition of bis(2-oxo-3-oxazolidinyl)phosphinic chloride (168.6 mg, 0.660 mmol). After 60 min, acethydrazide (122.7 mg, 1.66 mmol) was added and the mixture was heated to 66° C. for 3 h. The reaction was quenched by addition of water (10 mL) and the mixture extracted with ethyl acetate (3×30 mL). The combined organic layers were washed with water, dried (Na₂SO₄) and concentrated in vacuo. The residue was purified by preparative HPLC (Gemini-C18 acetonitrile/water (with 0.1% trifluoroacetic acid) 20-50%), followed by chiral SFC (Chiralpak-IC, ethanol) to afford the enantiopure (−)-title compound (45 mg, 34%) as a white amorphous freeze-dried solid. MS m/z: 401.2 ([M+H]⁺), ESI pos.

### Example 53

### (7S,15S)-9-(2,6-difluorophenyl)-N-(2-hydroxyethyl)-7,15-dimethyl-13,16-dioxa-18-thia-2,3,5,8-tetrazatetracyclo[8.8.0.02,6.011,17]octadeca-1(10),3,5,8,11(17)-pentaene-4-carboxamide

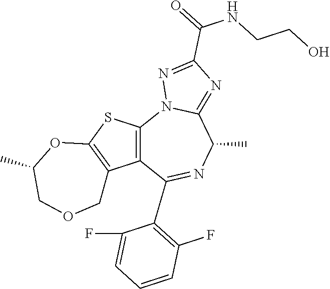 [SMILES: C[C@@H]1N=C(c2c(F)cccc2F)c2c(sc3c2COC[C@H](C)O3)-n2nc(C(=O)NCCO)nc21]

### a) ethyl (7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaene-4-carboxylate

The title compound was obtained in analogy to Example 22 a and b, using (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N) instead of (13S)-15-(2,6-difluorophenyl)-13-methyl-4,7-dioxa-9-thia-11,14-diazatricyclo[8.5.0.02,8]pentadeca-1(10),2(8),14-triene-12-one (Building block K), as a yellow solid. MS m/z: 474.6 ([M+H]⁺), ESI pos.

### b) (7S,15S)-9-(2,6-difluorophenyl)-N-(2-hydroxyethyl)-7,15-dimethyl-13,16-dioxa-18-thia-2,3,5,8-tetrazatetracyclo[8.8.0.02,6.011,17]octadeca-1(10),3.5.8.11(17)-pentaene-4-carboxamide

To a solution of ethyl (7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaene-4-carboxylate (100 mg, 0.210 mmol) in ethanol (5 mL) was added 2-hydroxyethylamine (0.06 mL, 1.05 mmol). The reaction mixture was stirred for 15 h at room temperature. The crude product was purified directly by preparative HPLC (Gemini C18, acetonitrile/water with 0.1% trifluoroacetic acid) to afford the title compound (46.5 mg, 45%) as a yellow solid. MS m/z: 490 ([M+H]⁺), ESI pos. ¹H NMR (CDCl₃, 400 MHz) δ ppm: 7.62 (dd, J=7.6, 4.0 Hz, 1H), 7.35 (tt, J=8.4, 6.2 Hz, 1H), 7.00-6.77 (m, 2H), 4.33 (td, J=6.1, 2.0 Hz, 1H), 4.24-4.12 (m, 2H), 3.99 (dt, J=10.3, 5.5 Hz, 1H), 3.89-3.78 (m, 3H), 3.72-3.60 (m, 2H), 3.52 (dd, J=13.6, 7.9 Hz, 1H), 2.07 (d, J=6.7 Hz, 3H), 1.30 (dd, J=6.6, 1.9 Hz, 3H).

### Example 54

### (7S,16R)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene

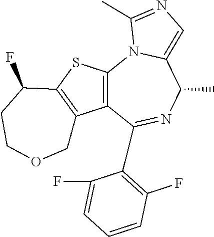 [SMILES: Cc1ncc2n1-c1sc3c(c1C(c1c(F)cccc1F)=N[C@H]2C)COCC[C@H]3F]

### a) (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaen-16-one

To a solution of (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaene (170 mg, 0.420 mmol) in acetonitrile (16 mL) was added copper(II) sulfate pentahydrate (424 mg, 1.7 mmol), potassium persulfate (459 mg, 1.7 mmol) and water (16 mL). The resulting mixture was stirred at 50° C. for 7 h, before being diluted with ethylacetate. The organic phase was washed with water (2×50 mL), dried (Na₂SO₄), filtered and concentrated to afford the title compound (146 mg, 83%) as a light yellow solid, which was used in the next step without further purification. MS m/z: 415.1 ([M+H]⁺), ESI pos.

### b) (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaen-16-ol

To a solution of (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaen-16-one (146 mg, 0.350 mmol) in methanol (8 mL) was added lithium borohydride (23 mg, 1.06 mmol). The resulting mixture was stirred at 25° C. for 1 h. The mixture was concentrated in vacuo and purified by flash column chromatography (silica, dichloromethane/methanol 96:4) to afford the title compound (123 mg, 84%) as a light yellow solid. MS m/z: 417.2 ([M+H]⁺), ESI pos.

### c) (7S,16R)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene

To a cooled solution (0° C.) of (7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaen-16-ol (123 mg, 0.3 mmol) in dichloromethane (5 mL) was added bis(2-methoxyethyl)aminosulfur trifluoride (98 mg, 0.44 mmol) and the resulting mixture was allowed to warm to room temperature and stirred for 3 h. The reaction was quenched by addition of water (5 mL), and extracted with dichloromethane (3×10 mL). The combined organic layers were washed with water, dried (Na₂SO₄), filtered and concentrated in vacuo. The residue was purified by preparative HPLC (Gemini-C18, acetonitrile/water (with 0.1% trifluoroacetic acid) 30-50%), followed by chiral SFC (Chiralpak-IC, ethanol/DEA) to afford the enantiopure (−)-title compound (21.5 mg, 17%) as a white amorphous freeze-dried solid. MS m/z: 419.2 ([M+H]⁺), ESI pos. ¹H NMR (CDCl₃, 400 MHz) δ ppm: 7.38 (tt, J=8.5, 6.3 Hz, 1H), 6.93 (t, J=8.3 Hz, 2H), 5.77 (ddd, J=48.3, 7.6, 2.9 Hz, 1H), 4.44 (d, J=15.2 Hz, 1H), 4.36 (q, J=6.7 Hz, 1H), 4.05 (ddd, J=12.3, 7.1, 5.0 Hz, 1H), 3.93 (ddd, J=10.1, 6.2, 2.0 Hz, 1H), 3.88 (dd, J=15.2, 1.3 Hz, 1H), 2.73 (s, 3H), 2.45-2.28 (m, 2H), 2.13 (d, J=6.8 Hz, 3H).

### Example 55

### (7S,16S)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene

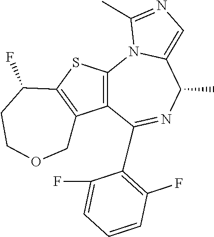 [SMILES: Cc1ncc2n1-c1sc3c(c1C(c1c(F)cccc1F)=N[C@H]2C)COCC[C@@H]3F]

In analogy to experiment of example 54 c, the enantiopure (−)-title compound (19 mg, 15%) as a white amorphous freeze-dried solid. MS m/z: 419.2 ([M+H]⁺), ESI pos. ¹H NMR (CDCl₃, 400 MHz) δ ppm: 7.37 (tt, J=8.4, 6.3 Hz, 1H), 6.92 (t, J=8.4 Hz, 2H), 5.97-5.76 (m, 1H), 4.48-4.33 (m, 2H), 4.18-4.09 (m, 1H), 3.92 (dd, J=15.2, 1.8 Hz, 1H), 3.87-3.80 (m, 1H), 2.72 (s, 3H), 2.43-2.30 (m, 2H), 2.13 (d, J=6.8 Hz, 3H).

NOTE: the assignment of the absolute configuration at C-16 (see name) in Example 54 and Example 55 was done arbitrarily, both in drawing and naming of the compounds.

### Example 56

### (7S,14R)-9-(2,6-difluorophenyl)-14-fluoro-3,7-dimethyl-16-oxa-18-thia-2,4,5,8-tetrazatetracyclo[8.8.0.02,6.011,17]octadeca-1(10),3,5,8,11(17)-pentaene

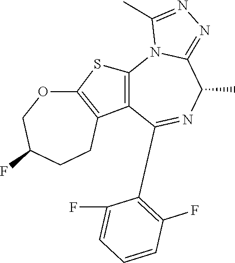 [SMILES: Cc1nnc2n1-c1sc3c(c1C(c1c(F)cccc1F)=N[C@H]2C)CC[C@@H](F)CO3]

### a) (7S)-14-benzyloxy-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaene

To a solution of (5S)-13-benzyloxy-3-(2,6-difluorophenyl)-5-methyl-11-oxa-9-thia-4,7-diazatricyclo[8.5.0.02,8]pentadeca-1(10),2(8),3-trien-6-one (Building block S, 100 mg, 0.210 mmol) in anhydrous tetrahydrofuran (3 mL) at 0° C., was added sodium hydride (60% dispersion in mineral oil, 17 mg, 0.430 mmol). After 10 min, bis(2-oxo-3-oxazolidinyl) phosphonic chloride (108.7 mg, 0.430 mmol) was added and the reaction mixture was stirred at 0° C. for 2 h. Finally, acethydrazide (79 mg, 1.07 mmol) was added and stirring was continued for another 0.5 h. The reaction mixture was heated to 60° C. for 12 h, before being quenched by addition of water (15 mL). The mixture was extracted with ethyl acetate (4×25 mL). The combined organic layers were washed with brine (2×25 mL) and concentrated in vacuo. The residue was purified by flash column chromatography (silica, 0-10% methanol in dichloromethane) to afford the title compound (50 mg, 46%) as a yellow solid. MS m/z: 507 ([M+H]⁺), ESI pos.

### b) (7S)-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaen-14-ol

To a solution of (7S)-14-benzyloxy-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaene (600 mg, 1.18 mmol) in dichloromethane (10 mL), was added boron tribromide (742 mg, 2.96 mmol) at 0° C. The reaction was stirred at 0° C. for 6 h, then quenched with methanol (10 mL). The mixture was concentrated in vacuo. The residue was purified by flash column chromatography (silica, 0-10% methanol in dichloromethane) to afford the title compound (400 mg, 81%) as a yellow solid. MS m/z: 417 ([M+H]⁺), ESI pos.

### c) (7S,14R)-9-(2,6-difluorophenyl)-14-fluoro-3,7-dimethyl-16-oxa-18-thia-2,4,5,8-tetrazatetracyclo[8.8.0.02,6.011,17]octadeca-1(10),3,5,8,11(17)-pentaene

To a solution of (7S)-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaen-14-ol (400 mg, 0.960 mmol) in dichloromethane (50 mL), was added dimethylaminosulfur trifluoride (773 mg, 4.8 mmol) at 0° C. dropwise. The solution was stirred at 0° C. for 12 h, then quenched by addition of water (25 mL). The mixture was extracted with dichloromethane (3×50 mL). The combined organic layers were dried (Na₂SO₄) and concentrated in vacuo. The residue was purified by preparative HPLC (Gemini-C18, acetonitrile/water (with 0.1% trifluoroacetic acid) 30-60%), followed by chiral SFC (chiral OZ—H, 35% methanol) to afford the enantiopure (−)-title compound (54 mg, 13%) as a white solid. MS m/z: 419 ([M+H]⁺), ESI pos. ¹H NMR (CDCl₃, 400 MHz) δ ppm: 7.40-7.31 (m, 1H), 6.93 (s, 2H), 5.02-4.66 (m, 1H), 4.52-4.05 (m, 3H), 2.69 (s, 3H), 2.64-2.15 (m, 2H), 2.12 (d, J=6.7 Hz, 3H), 2.08-1.69 (m, 3H).

### Example 57

### (7S)-9-(2,6-difluorophenyl)-4,7-dimethyl-13-oxa-18-thia-2,3,5,8-tetrazatetracyclo[8.8.0.02,6.011,17]octadeca-1(10),3,5,8,11(17)-pentaene

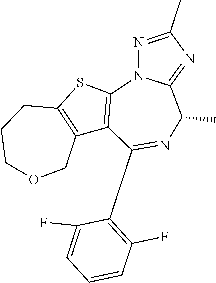 [SMILES: Cc1nc2n(n1)-c1sc3c(c1C(c1c(F)cccc1F)=N[C@H]2C)COCCC3]

The enantiopure (−)-title compound was obtained in analogy to Example 39, using (5S)-3-(2,6-difluorophenyl)-5-methyl-14-oxa-9-thia-4,7-diazatricyclo[8.5.0.02,8]pentadeca-1(10),2(8),3-trien-6-one (Building block R) instead of (6S,13S)-15-(2,6-difluorophenyl)-6,13-dimethyl-4,7-dioxa-9-thia-11,14-diazatricyclo[8.5.0.02,8]pentadeca-1(10),2(8),14-trien-12-one (Building block N) in step a) as an off-white amorphous freeze-dried solid. MS m/z: 401.1 ([M+H]⁺), ESI pos.

### Example 58

### (7S)-9-(2,6-difluorophenyl)-4,7-dimethyl-13-oxa-18-thia-2,5,8-triazatetracyclo[8.8.0.02,6.011,17]octadeca-1(10),3,5,8,11(17)-pentaene

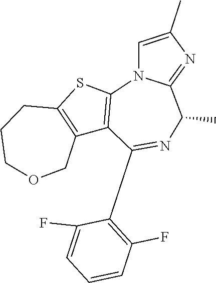 [SMILES: Cc1cn2c(n1)[C@H](C)N=C(c1c(F)cccc1F)c1c-2sc2c1COCCC2]

### a) 2-[[(5S)-3-(2,6-difluorophenyl)-5-methyl-14-oxa-9-thia-4,7-diazatricyclo[8.5.0.02,8]pentadeca-1(10),2(8),3-trien-6-ylidene]amino]propan-1-ol

To a solution of (5S)-3-(2,6-difluorophenyl)-5-methyl-14-oxa-9-thia-4,7-diazatricyclo[8.5.0.02,8]pentadeca-1(10),2(8),3-trien-6-one (Building block R, 79.6 mg, 0.220 mmol) in anhydrous tetrahydrofuran (4 mL) was added sodium hydride (60% dispersion in mineral oil, 13.25 mg, 0.330 mmol) at 0° C. under a nitrogen atmosphere. After 30 min, bis(2-oxo-3-oxazolidinyl)phosphinic chloride (112.4 mg, 0.440 mmol) was added and the resulting mixture was stirred at 0° C. for 60 min. Finally, DL-alaninol (66.3 mg, 0.880 mmol) was added and the reaction mixture was stirred at room temperature for 2.5 h. The reaction was quenched by addition of water (10 mL), and the mixture extracted with ethyl acetate (3×20 mL). The combined organic layers were washed with water, dried (Na₂SO₄) and concentrated in vacuo. The residue was purified by flash column chromatography (silica, dichloromethane/methanol 95:5) to afford the title compound (57 mg, 62%) as a yellow solid. MS m/z: 420.0 ([M+H]⁺), ESI pos.

### b) (7S)-9-(2,6-difluorophenyl)-4,7-dimethyl-13-oxa-18-thia-2.5,8-triazatetracyclo[8.8.0.02,6.011,17]octadeca-1(10),3,5,8,11(17)-pentaene

To a solution of 2-[[(5S)-3-(2,6-difluorophenyl)-5-methyl-14-oxa-9-thia-4,7-diazatricyclo[8.5.0.02,8]pentadeca-1(10),2(8),3-trien-6-ylidene]amino]propan-1-ol (57 mg, 0.140 mmol) in dichloromethane (2 mL) was added sodium hydrogencarbonate (45.7 mg, 0.540 mmol), followed by Dess-Martin periodinane (86.5 mg, 0.200 mmol) in three portions over 3 min. The suspension was stirred for 15 h, before being quenched by a slow addition of a saturated aqueous sodium hydrogen carbonate until no more gas evolution was observed. The mixture was diluted with dichloromethane (20 mL) and the organic phase washed with water (3×10 mL), dried (Na₂SO₄) and concentrated in vacuo. The residue was purified by flash column chromatography (silica, petroleum ether/ethyl acetate 1:1), followed by preparative HPLC (Gemini-C18, acetonitrile/water (with 0.1% trifluoroacetic acid) 30-60%) and, finally, by chiral SFC (Chiralpak-IC, EtOH) to afford the enantiopure (−)-title compound (0.8 mg, 1.4%) as an off-white amorphous freeze-dried solid. MS m/z: 400.1 ([M+H]⁺), ESI pos.

### Example 59

### (7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide

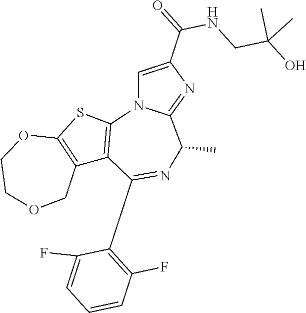 [SMILES: C[C@@H]1N=C(c2c(F)cccc2F)c2c(sc3c2COCCO3)-n2cc(C(=O)NCC(C)(C)O)nc21]

### a) (13S)-15-(2,6-difluorophenyl)-13-methyl-4,7-dioxa-9-thia-11,14-diazatricyclo[8.5.0.02,8]pentadeca-1(10),2(8),14-trien-12-imine

A solution of ammonia (7.0 M in methanol, 3.19 mL, 22.3 mmol) and (13S)-15-(2,6-difluorophenyl)-13-methyl-4,7-dioxa-9-thia-11,14-diazatricyclo[8.5.0.02,8] pentadeca-1(10),2(8),14-triene-12-thione (Building block L, 170 mg, 0.45 mmol) in methanol (3.2 mL) was placed in a sealed tube under a nitrogen atmosphere. The reaction mixture was heated at 60° C. and stirred for 5 h, before being concentrated in vacuo. The crude residue was purified by flash column chromatography to afford the title compound (115 mg, 71%) as a yellow powder. MS m/z: 364.1 ([M+H]⁺), ESI pos.

### b) ethyl 3-[(13S)-15-(2,6-difluorophenyl)-12-imino-13-methyl-4,7-dioxa-9-thia-11,14-diazatricyclo[8.5.0.02,8]pentadeca-1(10),2(8),14-trien-11-yl]-2-oxo-propanoate

To a solution of (13S)-15-(2,6-difluorophenyl)-13-methyl-4,7-dioxa-9-thia-11,14-diazatricyclo[8.5.0.02,8]pentadeca-1(10),2(8),14-trien-12-imine (160 mg, 0.44 mmol) and ethyl bromopyruvate (0.28 mL, 2.2 mmol) in tetrahydrofuran (3.0 mL) was added potassium carbonate (122 mg, 0.88 mmol). The reaction mixture was stirred at 55° C. for 16 h, before being quenched by addition of water (10 mL). The mixture was extracted with ethyl acetate (3×30 mL). The combined organic layers were washed with brine, dried (Na₂SO₄) and concentrated in vacuo to afford the title compound (130 mg, 60%), which was used in the next step without further purification. MS m/z: 477.7 ([M+H]⁺), ESI pos.

### c) ethyl (75)-9-(2,6-difluorophenyl)-7-methyl-13,16-dioxa-18-thia-2,5,8-triazatetracyclo[8.8.0.02,6.011,17]octadeca-1(10),3.5.8.11(17)-pentaene-4-carboxylate

To a solution of ethyl 3-[(13S)-15-(2,6-difluorophenyl)-12-imino-13-methyl-4,7-dioxa-9-thia-11,14-diazatricyclo[8.5.0.02,8]pentadeca-1(10),2(8),14-trien-11-yl]-2-oxo-propanoate (100 mg, 0.21 mmol) in acetic acid (3.0 mL) was added 4 Å molecular sieves (58 mg) and the mixture was heated to 80° C. After 5 h, the reaction mixture was concentrated in vacuo and diluted with ethyl acetate (20 mL). The mixture was neutralized with saturated aqueous sodium hydrogen carbonate. The aqueous layer was further extracted with ethyl acetate (10 mL). The combined organic layers were washed with brine (20 mL), dried (Na₂SO₄) and concentrated in vacuo. The residue was purified by flash column chromatography (silica, 0-50% ethyl acetate in petroleum ether) to afford the title compound (80 mg, 80%) as a dark brown solid. MS m/z: 459.7 ([M+H]⁺), ESI pos.

### d) (7S)-9-(2,6-difluorophenyl)-7-methyl-13,16-dioxa-18-thia-2,5,8-triazatetracyclo[8.8.0.02,6.011,17]octadeca-1(10),3,5,8,11(17)-pentaene-4-carboxylic acid

To a solution of ethyl (7S)-9-(2,6-difluorophenyl)-7-methyl-13,16-dioxa-18-thia-2,5,8-triazatetracyclo[8.8.0.02,6.011,17]octadeca-1(10),3,5,8,11(17)-pentaene-4-carboxylate (120 mg, 0.26 mmol) in ethanol (3 mL) was added lithium hydroxide (2.0 M, 0.52 mL, 1.04 mmol). The mixture was stirred at room temperature for 10 h, then acidified with acetic acid to pH=4-5 and extracted with ethyl acetate (2×30 mL). The combined organic layers were dried (Na₂SO₄) and concentrated in vacuo to afford the title compound (90 mg, 80%) as a light yellow solid. MS m/z: 432.0 ([M+H]⁺), ESI pos.

### e) (7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide

To a solution of (7S)-9-(2,6-difluorophenyl)-7-methyl-13,16-dioxa-18-thia-2,5,8-triazatetracyclo[8.8.0.02,6.011,17]octadeca-1(10),3,5,8,11(17)-pentaene-4-carboxylic acid (100 mg, 0.23 mmol) and 1-amino-2-methylpropan-2-ol (62 mg, 0.70 mmol) in dichloromethane (5 mL) was added propylphosphonic anhydride (442 mg, 0.70 mmol) and DIPEA (150 mg, 1.16 mmol). The mixture was stirred at room temperature for 14 h, then diluted with dichloromethane (20 mL). The organic phase was washed with water (10 mL). The aqueous layer was further extracted with dichloromethane (3×10 mL). The combined organic layers were washed with brine (50 mL), dried (Na₂SO₄) and concentrated in vacuo. The residue was purified by flash column chromatography (silica, petroleum ether/ethyl acetate 5:1 to 1:5), followed by chiral SFC (Chiralpak-IC, EtOH) to afford the enantiopure (−)-title compound (33.7 mg, 28%) as a white solid. MS m/z: 502.9 ([M+H]⁺), ESI pos.

### Example 60

### (7S)-9-(2,6-difluorophenyl)-N-[(1-hydroxycyclopropyl)methyl]-7-methyl-13,16-dioxa-18-thia-2,3,5,8-tetrazatetracyclo[8.8.0.02,6.011,17]octadeca-1(10),3,5,8,11(17)-pentaene-4-carboxamide

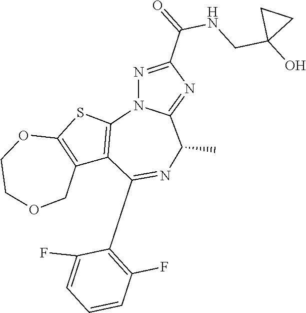 [SMILES: C[C@@H]1N=C(c2c(F)cccc2F)c2c(sc3c2COCCO3)-n2nc(C(=O)NCC3(O)CC3)nc21]

The title compound was obtained in analogy to Example 53, using 1-(aminomethyl)-cyclopropanol instead of 2-hydroxyethylamine in step b), as a light yellow solid. MS m/z: 501.9 ([M+H]⁺), ESI pos.

### Example 61

### (7S)-9-(2-chloro-6-fluoro-phenyl)-3,7-dimethyl-13-oxa-18-thia-2,4,5,8-tetrazatetracyclo[8.8.0.02,6.011,17]octadeca-1(10),3,5,8,11(17)-pentaene

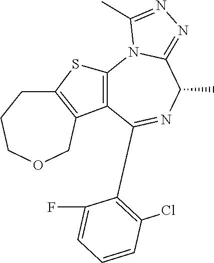 [SMILES: Cc1nnc2n1-c1sc3c(c1C(c1c(F)cccc1Cl)=N[C@H]2C)COCCC3]

The enantiopure (−)-title compound was obtained in analogy to Example 52, using (5S)-3-(2-chloro-6-fluoro-phenyl)-5-methyl-14-oxa-9-thia-4,7-diazatricyclo[8.5.0.02,8]pentadeca-1(10),2(8),3-trien-6-one (Building block T) instead of (5S)-3-(2,6-difluorophenyl)-5-methyl-14-oxa-9-thia-4,7-diazatricyclo[8.5.0.02,8]pentadeca-1(10),2(8),3-trien-6-one (Building block R), as a white amorphous freeze-dried solid. MS m/z: 417.0 ([{³⁵Cl}M+H]⁺), 419.0 ([{³⁷Cl}M+H]⁺), ESI pos.

### Example 62

### (7S)-9-(2,6-difluorophenyl)-N-[(1-hydroxycyclopropyl)methyl]-7-methyl-13-oxa-18-thia-2,3,5,8-tetrazatetracyclo[8.8.0.02,6.011,17]octadeca-1(10),3,5,8,11(17)-pentaene-4-carboxamide

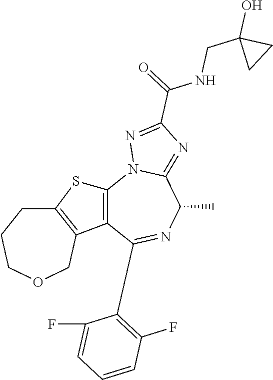 [SMILES: C[C@@H]1N=C(c2c(F)cccc2F)c2c(sc3c2COCCC3)-n2nc(C(=O)NCC3(O)CC3)nc21]

### a) (5S)-7-amino-3-(2,6-difluorophenyl)-5-methyl-14-oxa-9-thia-4,7-diazatricyclo[8.5.0.02,8]pentadeca-1(10),2(8),3-trien-6-one

To a solution of (5S)-3-(2,6-difluorophenyl)-5-methyl-14-oxa-9-thia-4,7-diazatricyclo[8.5.0.02,8]pentadeca-1(10),2(8),3-trien-6-one (Building block R, 1.5 g, 4.3 mmol) and O-(diphenylphosphinyl)hydroxylamine (1.2 g, 5.1 mmol) in N,N-dimethylformamide (20 mL) was added cesium carbonate (2.1 g, 6.4 mmol) at 0° C. The mixture was allowed to warm to room temperature and stirred for 4 h. The mixture was diluted with ethyl acetate (100 mL). The organic layer was washed with water (3×50 mL), dried (Na₂SO₄) and concentrated in vacuo. The residue was purified by flash column chromatography (silica, petroleum ether/ethyl acetate 5:1 to 1:1) to afford the title compound (770 mg, 52%) as a light yellow solid. MS m/z: 378.0 ([M+H]⁺), ESI pos.

### b) ethyl (7S)-9-(2,6-difluorophenyl)-7-methyl-13-oxa-18-thia-2.3.5,8-tetrazatetracyclo[8.8.0.02,6.011,17]octadeca-1(10),3,5,8,11(17)-pentaene-4-carboxylate

To a solution of (5S)-7-amino-3-(2,6-difluorophenyl)-5-methyl-14-oxa-9-thia-4,7-diazatricyclo[8.5.0.02,8]pentadeca-1(10),2(8),3-trien-6-one (770 mg, 2.0 mmol) in toluene (30 mL) was added ethyl 2-ethoxy-2-imino-acetate (736 mg, 2.5 mmol) and acetic acid (244 mg, 0.232 mL, 4.07 mmol). The mixture was heated to 100° C. under nitrogen and stirred for 22 h. After cooling down to room temperature, the mixture was diluted with ethyl acetate (20 mL). The organic phase was washed with water (10 mL). The aqueous layer was further extracted with ethyl acetate (3×50 mL). The combined organic layers were washed with brine (50 mL), dried (Na₂SO₄) and concentrated in vacuo. The residue was purified by flash column chromatography (silica, petroleum ether/ethyl acetate 10:1 to 1:8) to afford the title compound (450 mg, 90% pure) as a yellow solid. MS m/z: 459.0 ([M+H]⁺), ESI pos.

### c) (7S)-9-(2,6-difluorophenyl)-N-[(1-hydroxycyclopropyl)methyl]-7-methyl-13-oxa-18-thia-2.3.5,8-tetrazatetracyclo[8.8.0.02,6.011,17]octadeca-1(10),3.5.8.11(17)-pentaene-4-carboxamide

To a solution of ethyl (7S)-9-(2,6-difluorophenyl)-7-methyl-13-oxa-18-thia-2,3,5,8-tetrazatetracyclo[8.8.0.02,6.011,17]octadeca-1(10),3,5,8,11(17)-pentaene-4-carboxylate (200 mg, 0.440 mmol) in ethanol (5 mL) was added 1-(aminomethyl)cyclopropanol (190 mg, 2.18 mmol) at room temperature under a nitrogen atmosphere. The mixture was stirred at room temperature for 15 h. The crude reaction was purified by flash column chromatography (silica, 0-80% ethyl acetate in petroleum ether), followed by chiral SFC (Chiralpak-AD, 35% methanol, 0.2% ammonium hydroxide) to afford the enantiopure (−)-title compound (67.5 mg, 31%) as an off-white solid. MS m/z: 500.2 ([M+H]⁺), ESI pos.

### Example 63

### (7S)-9-(2,6-difluorophenyl)-N-(trans-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide

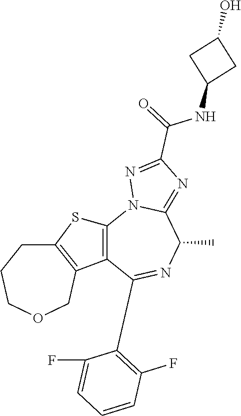 [SMILES: C[C@@H]1N=C(c2c(F)cccc2F)c2c(sc3c2COCCC3)-n2nc(C(=O)N[C@H]3C[C@H](O)C3)nc21]

To a solution of trans-3-aminocyclobutanol hydrochloride (269 mg, 2.18 mmol) in ethanol (10 mL) was added ethyl (7S)-9-(2,6-difluorophenyl)-7-methyl-13-oxa-18-thia-2,3,5,8-tetrazatetracyclo[8.8.0.02,6.011,17]octadeca-1(10),3,5,8,11(17)-pentaene-4-carboxylate (200 mg, 0.440 mmol) and triethylamine (1.32 g, 13.09 mmol) at room temperature under a nitrogen atmosphere. The mixture was stirred at 60° C. for 15 h. The crude reaction was purified directly by flash column chromatography (silica, 0-80% ethyl acetate in petroleum ether), followed by chiral SFC (Chiralpak WHELK-01 mm, 50% methanol) to afford the enantiopure (−)-title compound (43.5 mg, 20%) as an off-white solid. MS m/z: 500.1 ([M+H]⁺), ESI pos.

### Example 64

### (7S)-9-(2,6-difluorophenyl)-N-(cis-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide

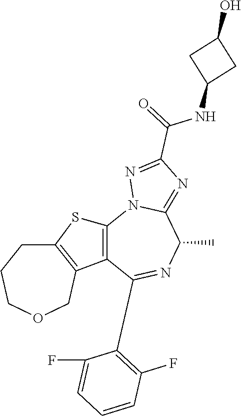 [SMILES: C[C@@H]1N=C(c2c(F)cccc2F)c2c(sc3c2COCCC3)-n2nc(C(=O)N[C@H]3C[C@@H](O)C3)nc21]

The title compound was obtained in analogy to Example 63, using cis-3-aminocyclobutanol hydrochloride instead of trans-3-aminocyclobutanol hydrochloride, as an off-white solid. MS m/z: 500.2 ([M+H]⁺), ESI pos.

### Example 65

### (7S)-9-(2,6-difluorophenyl)-N-[(1-hydroxycyclopropyl)methyl]-7-methyl-13,16-dioxa-18-thia-2,5,8-triazatetracyclo[8.8.0.02,6.011,17]octadeca-1(10),3,5,8,11(17)-pentaene-4-carboxamide

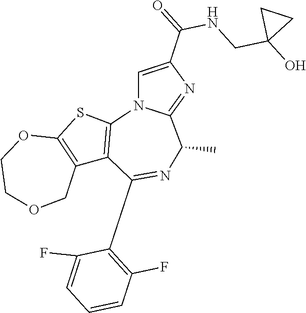 [SMILES: C[C@@H]1N=C(c2c(F)cccc2F)c2c(sc3c2COCCO3)-n2cc(C(=O)NCC3(O)CC3)nc21]

The title compound was obtained in analogy to Example 59, using 1-(aminomethyl)cyclopropanol instead of 1-amino-2-methylpropan-2-ol in step e), as a light yellow solid. MS m/z: 500.7 ([M+H]⁺), ESI pos.

### Example 66

### (7S)-9-(2,6-difluorophenyl)-N-(2-hydroxyethyl)-7-methyl-13-oxa-18-thia-2,5,8-triazatetracyclo[8.8.0.02,6.011,17]octadeca-1(10),3,5,8,11(17)-pentaene-4-carboxamide

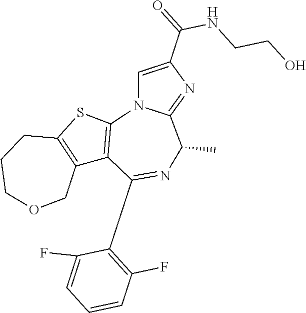 [SMILES: C[C@@H]1N=C(c2c(F)cccc2F)c2c(sc3c2COCCC3)-n2cc(C(=O)NCCO)nc21]

### a) (5S)-3-(2,6-difluorophenyl)-5-methyl-14-oxa-9-thia-4,7-diazatricyclo[8.5.0.02,8]pentadeca-1(10),2(8),3-triene-6-thione

A solution of (5S)-3-(2,6-difluorophenyl)-5-methyl-14-oxa-9-thia-4,7-diazatricyclo[8.5.0.02,8]pentadeca-1(10),2(8),3-trien-6-one (Building Block R, 2 g, 5.5 mmol) and pyridine (1.79 mL, 22.08 mmol) in dichloromethane (50 mL) was cooled to −40° C., before addition of trifluoromethanesulfonic anhydride (4.67 g, 16.6 mmol). The mixture was allowed to gradually warm to −5° C. over a period of 2 h, then stirred for an additional 2 h at room temperature. The reaction was re-cooled to −5° C., before addition of ammonium sulfide in water (5.64 g, 16.56 mmol). After 2 h, the crude reaction mixture was purified directly by flash column chromatography (silica, 0-80% ethyl acetate in petroleum ether) to afford the title compound (1.2 g, 57%) as a brown foam. MS m/z: 379 ([M+H]⁺), ESI pos.

### b) (5S)-3-(2,6-difluorophenyl)-5-methyl-14-oxa-9-thia-4,7-diazatricyclo[8.5.0.02,8]pentadeca-1(10),2(8),3-trien-6-imine

A solution of ammonia (7.0 M in methanol, 14.7 mL, 103 mmol) and (5S)-3-(2,6-difluorophenyl)-5-methyl-14-oxa-9-thia-4,7-diazatricyclo[8.5.0.02,8]pentadeca-1(10),2(8),3-triene-6-thione (780.0 mg, 2.06 mmol) in methanol (20 mL) was placed in a seal-tube under a nitrogen atmosphere. The reaction mixture was heated to 60° C. and stirred for 15 h. The mixture was concentrated in vacuo and purified by flash column chromatography (silica, 0-50% ethyl acetate in petroleum ether) to afford the title compound (740 mg, 99%) as a yellow powder. MS m/z: 362 ([M+H]⁺), ESI pos.

### c) ethyl (S)-3-(5-(2,6-difluorophenyl)-2-imino-3-methyl-2.3.6.8.9,10-hexahydro-1H-oxepino[3′0.4′:4.5]thieno[2,3-e][1,4]diazepin-1-yl)-2-oxopropanoate

To a solution of (5S)-3-(2,6-difluorophenyl)-5-methyl-14-oxa-9-thia-4,7-diazatricyclo[8.5.0.02,8]pentadeca-1(10),2(8),3-trien-6-imine (160 mg, 0.44 mmol) and ethyl bromopyruvate (0.28 mL, 2.2 mmol) in tetrahydrofuran (3.0 mL) was added potassium carbonate (121.7 mg, 0.880 mmol). The reaction mixture was stirred at 55° C. for 16 h, before being quenched by addition of water (5 mL). The mixture was extracted with ethyl acetate (2×30 mL). The combined organic layers were washed with brine, dried (Na₂SO₄) and concentrated in vacuo. The crude product (130 mg, 60%) was used in the next step without further purification. MS m/z: 476 ([M+H]⁺), ESI pos.

### d) ethyl (S)-6-(2,6-difluorophenyl)-4-methyl-7,9,10,11-tetrahydro-4H-imidazo[1,2-a]oxepino[3′,4′:4,5]thieno[3,2-f][1,4]diazepine-2-carboxylate

To a solution of ethyl (S)-3-(5-(2,6-difluorophenyl)-2-imino-3-methyl-2,3,6,8,9,10-hexahydro-1H-oxepino[3′,4′:4,5]thieno[2,3-e][1,4]diazepin-1-yl)-2-oxopropanoate (100 mg, 0.210 mmol) in acetic acid (3.0 mL) was added 4 Å molecular sieves (58 mg, 0.42 mmol). The reaction mixture was stirred at 80° C. for 5 h, before being concentrated in vacuo and diluted with ethyl acetate (20 mL). The mixture was neutralized with saturated aqueous sodium hydrogen carbonate. The aqueous layer was further extracted with ethyl acetate (10 mL). The combined organic layers were washed with brine (20 mL), dried (Na₂SO₄) and concentrated in vacuo. The residue was purified by flash column chromatography (silica, 0-50% ethyl acetate in petroleum ether) to afford the title compound (80 mg, 80%) as a dark brown solid. MS m/z: 458 ([M+H]⁺), ESI pos.

### e) (S)-6-(2,6-difluorophenyl)-4-methyl-7,9,10,11-tetrahydro-4H-imidazo[1,2-a]oxepino[3′,4′:4,5]thieno[3,2-f][1,4]diazepine-2-carboxylic acid

To a solution of ethyl (S)-6-(2,6-difluorophenyl)-4-methyl-7,9,10,11-tetrahydro-4H-imidazo[1,2-a]oxepino[3′,4′:4,5]thieno[3,2-f][1,4]diazepine-2-carboxylate (120 mg, 0.260 mmol) in ethanol (3 mL) was added aqueous lithium hydroxide (2.0 M, 0.52 mL, 1.04 mmol). The mixture was stirred at room temperature for 10 h, before addition of acetic acid to pH=4-5. The mixture was extracted with ethyl acetate (2×20 mL). The combined organic layers were dried (Na₂SO₄) and concentrated in vacuo to afford the title compound (90 mg, 80%) as a light yellow solid. MS m/z: 430 ([M+H]⁺), ESI pos.

### f) (7S)-9-(2,6-difluorophenyl)-N-(2-hydroxyethyl)-7-methyl-13-oxa-18-thia-2,5,8-triazatetracyclo[8.8.0.02,6.011,17]octadeca-1(10),3,5,8,11(17)-pentaene-4-carboxamide

To a solution of 2-hydroxyethylamine (0.04 mL, 0.70 mmol) and (7S)-9-(2,6-difluorophenyl)-7-methyl-13-oxa-18-thia-2,5,8-triazatetracyclo[8.8.0.02,6.011,17]octadeca-1(10),3,5,8,11(17)-pentaene-4-carboxylic acid (150 mg, 0.350 mmol) in dichloromethane (5 mL) was added propylphosphonic anhydride (667 mg, 1.05 mmol) and DIPEA (225 mg, 1.75 mmol). The mixture was stirred at room temperature for 2 h, before being diluted with dichloromethane (20 mL). The organic phase was washed with water (10 mL). The aqueous layer was further extracted with dichloromethane (3×10 mL). The combined organic layers were washed with brine (50 mL), dried (Na₂SO₄) and concentrated in vacuo. The residue was purified by flash column chromatography (silica, petroleum ether/ethylacetate 5:1 to 1:5), followed by chiral SFC (Chiralpak-AD, 35% methanol, 0.2% ammonium hydroxide) to afford the title compound (45.5 mg, 27%) as an off-white solid. MS m/z: 473.0 ([M+H]⁺), ESI pos.

### Example 67

### (7S)-9-(2,6-difluorophenyl)-N-(2-hydroxy-2-methyl-propyl)-7-methyl-13-oxa-18-thia-2,5,8-triazatetracyclo[8.8.0.02,6.011,17]octadeca-1(10),3,5,8,11(17)-pentaene-4-carboxamide

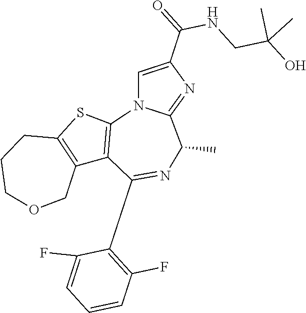 [SMILES: C[C@@H]1N=C(c2c(F)cccc2F)c2c(sc3c2COCCC3)-n2cc(C(=O)NCC(C)(C)O)nc21]

The title compound was obtained in analogy to Example 66, using 1-amino-2-methylpropan-2-ol instead of 2-hydroxyethylamine in step f), as an off-white solid. MS m/z: 501.0 ([M+H]⁺), ESI pos.

### Example 68

### (7S)-9-(2,6-difluorophenyl)-N-[(1-hydroxycyclopropyl)methyl]-7-methyl-13-oxa-18-thia-2,5,8-triazatetracyclo[8.8.0.02,6.011,17]octadeca-1(10),3,5,8,11(17)-pentaene-4-carboxamide

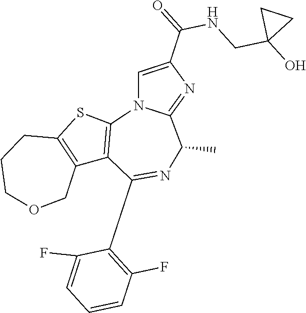 [SMILES: C[C@@H]1N=C(c2c(F)cccc2F)c2c(sc3c2COCCC3)-n2cc(C(=O)NCC3(O)CC3)nc21]

The title compound was obtained in analogy to Example 66, using 1-(aminomethyl)-cyclopropanol instead of 2-hydroxyethylamine in step f), as an off-white solid. MS m/z: 499.0 ([M+H]⁺), ESI pos.

### Example 69

### (7S)-9-(2,6-difluorophenyl)-7-methyl-N-[(2S)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide

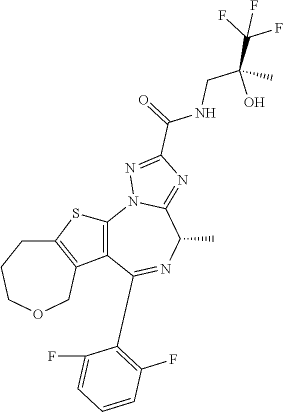 [SMILES: C[C@@H]1N=C(c2c(F)cccc2F)c2c(sc3c2COCCC3)-n2nc(C(=O)NC[C@](C)(O)C(F)(F)F)nc21]

The title compound was obtained in analogy to Example 22, using ethyl (7S)-9-(2,6-difluorophenyl)-7-methyl-13-oxa-18-thia-2,3,5,8-tetrazatetracyclo[8.8.0.02,6.011,17]octadeca-1(10),3,5,8,11(17)-pentaene-4-carboxylate instead of ethyl (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylate in step c) and (2S)-3-amino-1,1,1-trifluoro-2-methyl-propan-2-ol instead of azetidine in step d), as a white solid. MS m/z: 556.0 ([M+H]⁺), ESI pos.

### Example 70

### (7S)-9-(2,6-difluorophenyl)-7-methyl-N-[(2R)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide

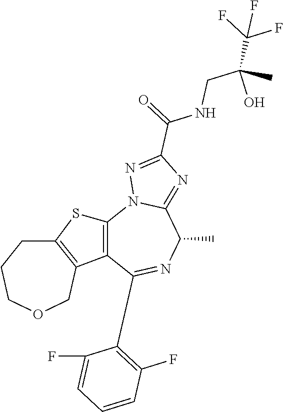 [SMILES: C[C@@H]1N=C(c2c(F)cccc2F)c2c(sc3c2COCCC3)-n2nc(C(=O)NC[C@@](C)(O)C(F)(F)F)nc21]

The title compound was obtained in analogy to Example 22, using ethyl (7S)-9-(2,6-difluorophenyl)-7-methyl-13-oxa-18-thia-2,3,5,8-tetrazatetracyclo[8.8.0.02,6.011,17]octadeca-1(10),3,5,8,11(17)-pentaene-4-carboxylate instead of ethyl (7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxylate in step c) and (2R)-3-amino-1,1,1-trifluoro-2-methyl-propan-2-ol instead of azetidine in step d), as a white solid. MS m/z: 556.0 ([M+H]⁺), ESI pos.

### Example 71

### (7S)-9-(2,6-difluorophenyl)-N-(2-hydroxy-2-methyl-propyl)-7-methyl-13-oxa-18-thia-2,3,5,8-tetrazatetracyclo[8.8.0.02,6.011,17]octadeca-1(10),3,5,8,11(17)-pentaene-4-carboxamide

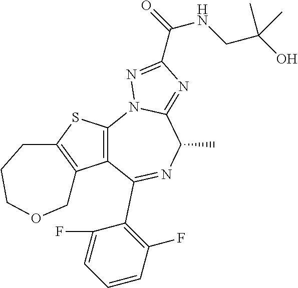 [SMILES: C[C@@H]1N=C(c2c(F)cccc2F)c2c(sc3c2COCCC3)-n2nc(C(=O)NCC(C)(C)O)nc21]

The title compound was obtained in analogy to Example 62, using 1-amino-2-methylpropan-2-ol instead of 1-(aminomethyl)-cyclopropanol in step c), as a white solid. MS m/z: 502.0 ([M+H]⁺), ESI pos.

### Reference Compound RE-B

### (7S)-9-(2,6-difluorophenyl)-3,7-dimethyl-17-thia-2,4,5,8-tetrazatetracyclo[8.7.0.02,6.011,16]heptadeca-1(10),3,5,8,11(16)-pentaene

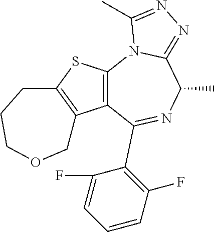 [SMILES: Cc1nnc2n1-c1sc3c(c1C(c1c(F)cccc1F)=N[C@H]2C)COCCC3]

### a) (2-amino-5,6-dihydro-4H-cyclohexan[b]thiophen-3-yl)-(2,6-difluorophenyl)methanone

In analogy to experiment of building block A a, cyclohexanone was converted into the title compound (691 mg, 52%) which was obtained as a yellow foam. MS: 294.1 ([M+H]⁺), ESI pos.

### b) tert-butyl N-[(1S)-2-[[3-(2,6-difluorobenzoyl)-5,6-dihydro-4H-cyclohexan[b]thiophen-2-yl]amino]-1-methyl-2-oxo-ethyl]carbamate

In analogy to experiment of building block C a, (2-amino-5,6-dihydro-4H-cyclohexan[b]thiophen-3-yl)-(2,6-difluorophenyl)methanone was converted into the title compound (734 mg, 78%) which was obtained as a yellow foam. MS: 463.3 ([M−H]⁻), ESI neg.

### c) (2S)-2-amino-N-[3-(2,6-difluorobenzoyl)-5,6-dihydro-4H-cyclohexan[b]thiophen-2-yl]propanamide

To an ice-cooled mixture of tert-butyl N-[(1S)-2-[[3-(2,6-difluorobenzoyl)-5,6-dihydro-4H-cyclohexan[b]thiophen-2-yl]amino]-1-methyl-2-oxo-ethyl]carbamate (734 mg, 1.58 mmol) in dichloromethane (11.4 mL) was added hydrochloric acid (4.0 M in 1,4-dioxane, 1.98 mL, 7.9 mmol). The reaction mixture was stirred at room temperature for 3.5 h then quenched with saturated aqueous sodium hydrogen carbonate. The aqueous layer was extracted with ethyl acetate (2×35 mL). The combined organic layers were washed with brine (2×15 mL), dried (Na₂SO₄) and concentrated in vacuo to afford the title compound (606 mg, quantitative) as a green solid. MS: 365.2 ([M+H]⁺), ESI pos.

### d) (3S)-5-(2,6-difluorophenyl)-3-methyl-1.3.6.7.8,9-hexahydrobenzothiopheno[2,3-e][1,4]diazepin-2-one

In analogy to experiment of building block A d, (2S)-2-amino-N-[3-(2,6-difluorobenzoyl)-5,6-dihydro-4H-cyclohexan[b]thiophen-2-yl]propanamide was converted into the title compound (371 mg, 64%) which was obtained as a yellow solid. MS: 347.2 ([M+H]⁺), ESI pos.

### e) (3S)-5-(2,6-difluorophenyl)-3-methyl-1.3.6.7.8,9-hexahydrobenzothiopheno[2,3-e][1,4]diazepine-2-thione

In analogy to experiment of building block D, (3S)-5-(2,6-difluorophenyl)-3-methyl-1,3,6,7,8,9-hexahydrobenzothiopheno[2,3-e][1,4]diazepin-2-one was converted into the title compound (383 mg, 99%) which was obtained as an orange waxy solid. MS: 363.2 ([M+H]⁺), ESI pos.

### f) (7S)-9-(2,6-difluorophenyl)-3,7-dimethyl-17-thia-2,4,5,8-tetrazatetracyclo[8.7.0.02,6.011.16]heptadeca-1(10),3,5,8,11(16)-pentaene

A microwave vial was charged with (3S)-5-(2,6-difluorophenyl)-3-methyl-1,3,6,7,8,9-hexahydrobenzothiopheno[2,3-e][1,4]diazepine-2-thione (67 mg, 0.19 mmol), acetohydrazide (137 mg, 1.85 mmol) and butan-1-ol (3.79 mL). The vial was capped and heated in a microwave oven to 130° C. for 3 h. The reaction mixture was concentrated in vacuo, adsorbed on ISOLUTE® HM-N and purified by flash column chromatography (silica, 0 to 7% methanol in dichloromethane), followed by SFC (Reprosil Chiral-NR, methanol containing 0.2% diethyl amine) to afford the title compound (10.8 mg, 14%) as a light brown solid. MS: 385.3 ([M+H]⁺), ESI pos. ¹H NMR (CDCl₃, 300 MHz) δ: 7.43-7.28 (m, 1H), 7.07-6.71 (m, 2H), 4.40-4.25 (m, 1H), 2.82-2.72 (m, 2H), 2.68 (s, 3H), 2.34-2.20 (m, 1H), 2.16-2.05 (m, 3H), 1.99-1.84 (m, 1H), 1.81-1.49 (m, 4H).

### Assay Procedures

### Membrane Preparation and Binding Assay for γ1-Containing GABA_{A} Subtypes

The affinity of compounds at GABA_{A} γ1 subunit-containing receptors was measured by competition for [³H]RO7239181 (67.3 Ci/mmol; Roche) binding to membranes from HEK293F cells (ThermoFisher R79007) expressing human (transiently transfected) receptors of composition α5β2γ1, α2β2γ1, α1β2γ1. For better protein expression of the α2 subunit-containing receptors, the 28 amino acid long signal peptide (Met1 to Ala28) of the human GABA_{A} α2 subunit was substituted by the 31 amino acid long signal peptide (Met1 to Ser31) of human GABA_{A} α5 subunit.

Harvested pellets from HEK293F cells expressing the different GABA_{A} receptor subtypes were resuspended in Mannitol Buffer pH 7.2-7.4 (Mannitol 0.29 M, Triethylamine 10 mM, Acetic acid 10 mM, EDTA 1 mM plus protease inhibitors (20 tablets Complete, Roche Diagnostics Cat. No. 05 056 489 001 per liter)), washed two times and then resuspended at 1:10 to 1:15 dilution in the same buffer. Cell disruption was performed by stirring the suspension in a Parr vessel #4637 at 435 psi for 15 minutes, and then the suspensions were centrifuged at 1000×g for 15 minutes at 4° C. (Beckman Avanti J-HC; rotor JS-4.2). The supernatant (S1) was transferred in a 21 Schott flask and the pellet (P1) was resuspended with Mannitol Buffer up to 175 ml. The resuspended pellet was transferred into a 250 ml Corning centrifugal beaker and centrifuged at 1500×g for 10 minutes at 4° C. (Beckman Avanti J-HC; rotor JS-4.2). The supernatant (S1) was then transferred in the 21 Schott flask and the pellet was discarded. The supernatants (S1) were centrifuged in 500 ml Beckman polypropylene centrifugal beaker at 15'000×g for 30 minutes at 4° C. (Beckman Avanti J-20 XP; rotor JLA-10.500). The pellet (P2) was resuspended with Mannitol Buffer 1:1 and frozen at −80° C. The supernatant (S2) was centrifuged in 100 ml Beckman polypropylene centrifugal tubes at 48000×g for 50 minutes at 4° C. (Beckman Avanti J-20 XP; rotor JA-18). The supernatant (S3) was discarded and the pellet (P3) was resuspended with 1:1 Mannitol Buffer. The P2 and P3 protein concentration was determined with the BIORAD Standard assay method with bovine serum albumin as standard and measured on the NANO-Drop 1000. The membrane suspension was aliquots (500 μl per tube) and stored at −80° C. until required.

Membrane homogenates were resuspended and polytronised (Polytron PT1200E Kinematica AG) in Potassium Phosphate 10 mM, KCl 100 mM binding buffer at pH 7.4 to a final assay concentration determined with a previous experiment.

Radioligand binding assays were carried out in a volume of 200 μL (96-well plates) which contained 100 μL of cell membranes, [³H]RO7239181 at a concentration of 1.5 nM (α5β2γ1) or 20-30 nM (α1β2γ1, α2β2γ1) and the test compound in the range of [0.3-10000]×10⁻⁹ M. Nonspecific binding was defined by 10×10⁻⁶ (α5β2γ1) and 30×10⁻⁶ M RO7239181 and typically represented less than 5% (α5β2γ1) and less than 20% (α1β2γ1, α2β2γ1) of the total binding. Assays were incubated to equilibrium for 1 hour at 4° C. and then, membranes were filtered onto unifilter (96-well white microplate with bonded GF/C filters preincubated 20-50 minutes in 0.3% Polyethylenimine) with a Filtermate 196 harvester (Packard BioScience) and washed 4 times with cold Potassium Phosphate 10 mM pH 7.4, KCl 100 mM binding buffer. After anhydrousing, filter-retained radioactivity was detected by liquid scintillation counting. Kᵢ values were calculated using Excel-Fit (Microsoft) and are the means of two determinations.

The compounds of the accompanying examples were tested in the above described assays, and the preferred compounds were found to possess a Kᵢ value for the displacement of [³H]RO7239181 from GABA_{A} γ1 subunit-containing receptors (e.g. α5β2γ1, α2β2γ1, α1β2γ1) of 100 nM or less. Most preferred are compounds with a Ki (nM)<50. Representative test results, obtained by the above described assay measuring binding affinity to HEK293 cells expressing human (h) receptors, are shown in the Table 1.

### Preparation of [³H]RO7239181, 6-chloro-5-(2,6-difluorophenyl)-7-methyl-1-(tritritiomethyl)-3H-1,4-benzodiazepin-2-one

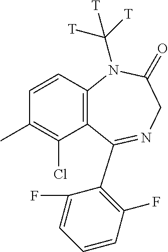

### a) 5-chloro-2-methyl-3,1-benzoxazin-4-one

A solution of 2-amino-6-chlorobenzoic acid (250 g, 1.46 mol) in acetic anhydride (1250 mL) was stirred at 140° C. for 2 h. The reaction mixture was concentrated in vacuo. The resulting crude residue was suspended in ethyl acetate (1000 mL), stirred for 30 min, filtered and dried in vacuo to afford the title compound (238 g, 84%) as a grey solid. ¹H NMR (DMSO-d6, 400 MHz): δ: 7.80 (app t, J=8.0 Hz, 1H), 7.62 (d, J=8.0 Hz, 1H), 7.49 (d, J=7.6 Hz, 1H), 2.36 (s, 3H).

### b) N-[3-chloro-2-(2,6-difluorobenzoyl)phenyl]acetamide

To a solution of 5-chloro-2-methyl-3,1-benzoxazin-4-one (100 g, 511.2 mmol) and 2-bromo-1,3-difluorobenzene (118.4 g, 613.5 mmol) in tetrahydrofuran (1000 mL) was added dropwise i-PrMgCl·LiCl (1.3 M, 500 mL, 650 mmol) at −70° C. under nitrogen. The mixture was allowed to warm up to room temperature within 1 h, quenched with saturated aqueous ammonium chloride (1500 mL) and extracted with ethyl acetate (2×1500 mL). The organic phase was washed with brine (2000 mL), dried (Na₂SO₄) and concentrated in vacuo. The residue was suspended in ethyl acetate (150 mL). The resulting suspension was stirred at room temperature for 20 min, filtered and dried in vacuo to afford the title compound (113 g, 71%) as an off-white solid. ¹H NMR (DMSO-d6, 400 MHz): δ: 9.85 (s, 1H), 7.65-7.45 (m, 1H), 7.40 (t, J=7.2 Hz, 1H), 7.38-7.34 (m, 2H), 7.16 (t, J=8.8 Hz, 2H), 1.85 (s, 3H).

### c) (2-amino-6-chloro-phenyl)-(2,6-difluorophenyl)methanone

To a solution of N-[3-chloro-2-(2,6-difluorobenzoyl)phenyl]acetamide (113 g, 364.9 mmol) in ethanol (250 mL) was added aqueous hydrochloric acid (12 M, 200 mL). The reaction mixture was stirred at 100° C. for 1 h, then diluted with ethyl acetate (1100 mL). The organic phase was washed with water (1100 mL), saturated aqueous sodium bicarbonate (1100 mL) and brine (1100 mL), dried over sodium sulfate and concentrated in vacuo. Petroleum ether (120 mL) was added to the crude and the suspension was stirred at room temperature for 20 min. The solid was filtered and dried to afford the title compound (88 g, 90%) as a yellow solid. ¹H NMR (DMSO-d6, 400 MHz): δ: 7.62-7.56 (m, 1H), 7.21-7.15 (m, 3H), 6.83 (d, J=7.6 Hz, 1H), 6.74 (s, 2H), 6.58 (d, J=7.6 Hz, 1H).

### d) (6-amino-3-bromo-2-chloro-phenyl)-(2,6-difluorophenyl)methanone

To a solution of (2-amino-6-chloro-phenyl)-(2,6-difluorophenyl)methanone (88.0 g, 328.8 mmol) in dichloromethane (225 mL) and N,N-dimethylformamide (225 mL) was added 1-bromopyrrolidine-2,5-dione (64.4 g, 362 mmol) at 0° C. The reaction mixture was stirred at 30° C. for 1 h. The mixture was diluted with dichloromethane (600 mL) and washed with water (500 mL) and brine (4×500 mL), dried (Na₂SO₄) and concentrated in vacuo. The residue was purified by chromatography (silica, petroleum ether/ethyl acetate, 1:0 to 2:1). The solid was suspended in petroleum ether (200 mL) and stirred at room temperature for 20 min. The suspension was filtered and the solid was dried in vacuo to afford the title compound (96.0 g, 84%) as a yellow solid. MS: 345.9 ([{⁷⁹Br, ³⁵Cl}M+H]⁺), 347.8 ([{⁸¹Br, ³⁵Cl or ⁷⁹Br, ³²Cl}M+H]⁺), ESI pos.

### e) 7-bromo-6-chloro-5-(2,6-difluorophenyl)-1,3-dihydro-1,4-benzodiazepin-2-one

To a solution of (6-amino-3-bromo-2-chloro-phenyl)-(2,6-difluorophenyl)methanone (25.0 g, 72.1 mmol) in pyridine (625 mL) was added ethyl 2-aminoacetate hydrochloride (70.5 g, 505 mmol). The reaction mixture was stirred at 135° C. for 36 h. The reaction mixture was concentrated in vacuo to remove pyridine. The residue was diluted with ethyl acetate (2000 mL) and washed with aqueous HCl (1.0 M, 3×1500 mL), water (2000 mL) and brine (2×1000 mL), dried (Na₂SO₄), filtered and concentrated in vacuo. The crude product was purified by flash column chromatography (silica, petroleum ether/ethyl acetate 10:1 to 2:1) to afford the title compound (10.1 g, 12%) as an off-white solid. MS: 385.0 ([{⁷⁹Br, ³⁵Cl}M+H]⁺), ESI pos.

### f) 6-chloro-5-(2,6-difluorophenyl)-7-methyl-1,3-dihydro-1,4-benzodiazepin-2-one

A microwave tube was charged with 7-bromo-6-chloro-5-(2,6-difluorophenyl)-1,3-dihydro-1,4-benzodiazepin-2-one (450 mg, 1.17 mmol), trimethylboroxine (205 mg, 228 μL, 1.63 mmol), potassium carbonate (242 mg, 1.75 mmol) and tetrakis(triphenylphosphine)palladium (0) (67.4 mg, 58.4 μmol). Degassed 1,4-dioxane (8.1 mL) and H₂O (2.7 ml) were added then the vial was capped. The suspension was reacted in microwave at 130° C. for 30 min to give complete conversion. The mixture was evaporated, treated with sat. aq. NaHCO₃ (20 mL) and extracted with EtOAc (2×20 mL). The organic layers were dried (Na₂SO₄), filtered and solvents were evaporated. The residue was purified by flash column chromatography (silica, 40 g, CH₂Cl₂/EtOAc in heptane 10% to 40% to 70%) to give the title compound (344 mg, 92%) as light yellow solid. MS (ESI): 321.1 ([M+H]⁺).

### g) 6-chloro-5-(2,6-difluorophenyl)-7-methyl-1-(tritritiomethyl)-3H-1,4-benzodiazepin-2-one

To a solution of [³H]methyl nosylate (1.85 GBq, 50 mCi, 0.61 μmol) in THF (200 μL) were added the N-desmethyl precursor 6-chloro-5-(2,6-difluorophenyl)-7-methyl-1,3-dihydro-1,4-benzodiazepin-2-one (0.43 mg, 1.34 μmol) dissolved in THF (200 μL) and 10 equivalents of sodium tert-butylate (0.5 M in THF, 13.4 μmol). After stirring for 4 h at room temperature the reaction mixture was treated with H₂O, evaporated, and the crude product was purified by HPLC (X-Terra Prep RP-18, 10×150 mm, MeCN/H₂O (containing 5% of MeCN) 40:60, 4 ml/min, 230 nm). The pure tritium-labeled compound was isolated by solid phase extraction (Sep-Pak Plus C18) and eluted from the cartridge as ethanolic solution to yield 1.6 GBq (43.2 mCi) of the target compound in >99% radio-chemical purity and a specific activity of 2.49 TBq/mmol (67.3 Ci/mmol) as determined by mass spectrometry (MS). The identity of the labeled compound was confirmed by HPLC (by co-injecting the unlabeled reference standard) and by MS.

MS: m/z=335 [M(H)+H]⁺ (16%), 337 [M(3H)+H]⁺ (0%), 339 [M(³H₂)+H]⁺ (16%), 341 [M(³H₃)+H]⁺ (68%).

### Membrane Preparation and Binding Assay for γ2-Containing GABA_{A} Subtypes

The affinity of compounds at GABA_{A} γ2 subunit-containing receptors was measured by competition for [³H]Flumazenil (81.1 Ci/mmol; Roche) binding to HEK293F cells expressing human (transiently transfected) receptors of composition α1β3γ2.

Harvested pellets from HEK293F cells expressing the different GABA_{A} γ2 receptor subtypes were resuspended in Mannitol Buffer pH 7,2-7,4 and processed as described above for the cells expressing the GABA_{A} γ1 subunit-containing receptors.

Radioligand binding assays were carried out in a volume of 200 μL (96-well plates) which contained 100 μL of cell membranes, [³H]Flumazenil at a concentration of 1 nM and the test compound in the range of [0.1·10⁻³−10]×10⁻⁶ M. Nonspecific binding was defined by 10⁻⁵ M Diazepam and typically represented less than 5% of the total binding. Assays were incubated to equilibrium for 1 hour at 4° C. and harvested onto GF/C uni-filters (Packard) by filtration using a Packard harvester and washing with ice-cold wash buffer (50 mM Tris; pH 7.5). After anhydrousing, filter-retained radioactivity was detected by liquid scintillation counting. Kᵢ values were calculated using Excel-Fit (Microsoft) and are the means of two determinations.

The compounds of the accompanying examples were tested in the above described assay, and the preferred compounds were found to possess large Kᵢ value for displacement of [³H]Flumazenil from the α1β3γ2 subtype of the human GABA_{A} receptor of 100 nM or above. Most preferred are compounds with a Kᵢ α1β3γ2 (nM) >300. In a preferred embodiment the compounds of the invention are binding selectively for the γ1 subunit-containing GABA_{A} receptors relative to γ2 subunit-containing GABA_{A} receptors. In particular, compounds of the present invention have γ2/γ1 selectivity ratio defined as “Kᵢ α1β3γ2 (nM)/Kᵢ α2β2γ1 (nM)” above 10-fold, or LogSel defined as “Log[Kᵢ α1β3γ2 (nM)/Kᵢ α2β2γ1 (nM)]” above 1. Representative test results, obtained by the above described assay measuring binding affinity to HEK293 cells expressing human (h) receptors, are shown in the Table 1 below.

TABLE 1

| | Ki | Ki | Ki | Ki | | |
|---|---|---|---|---|---|---|
| | h-GABA_{A} | h-GABA_{A} | h-GABA_{A} | h-GABA_{A} | γ2/γ1 | |
| | α5β2γ1 | α2β2γ1 | α1β2γ1 | α1β3γ2 | Selectivity | |
| Example | (nM) | (nM) | (nM) | (nM) | Ratio | LogSel |
| 1 | 1.40 | 7.5 | 20.0 | 373.5 | 49.6 | 1.70 |
| 2 | 1.70 | 7.4 | 19.2 | 994 | 133.9 | 2.13 |
| 3 | 3.69 | 26.6 | ND | 1359.9 | 51.0 | 1.71 |
| 4 | 8.65 | 47.8 | ND | 4521.1 | 94.6 | 1.98 |
| 5 | 10.5 | 63.5 | 245.5 | 1256.3 | 19.8 | 1.30 |
| 6 | 3.69 | 37.3 | ND | 2435.3 | 65.3 | 1.82 |
| 7 | 2.24 | 37.5 | ND | 7689.5 | 205.1 | 2.31 |
| 8 | 12.8 | 51.3 | 311.7 | 2758.1 | 53.8 | 1.73 |
| 9 | 2.79 | 14.3 | ND | 1134 | 79.4 | 1.90 |
| 10 | 2.72 | 18.2 | ND | 1159.7 | 63.6 | 1.80 |
| 11 | 2.70 | 17.4 | ND | 825 | 47.3 | 1.67 |
| 12 | 2.65 | 33.7 | 45.7 | 1008.4 | 29.9 | 1.48 |
| 13 | 5.67 | 15.1 | ND | 1192.7 | 78.9 | 1.90 |
| 14 | 4.87 | 38.4 | 117.8 | 10992.5 | 286.6 | 2.46 |
| 15 | 8.0 | 42.8 | 130.9 | 3531.6 | 82.5 | 1.92 |
| 16 | 13.6 | 89.8 | 421.2 | 1415.6 | 15.8 | 1.20 |
| 17 | 1.77 | 4.31 | 30.2 | 584.2 | 135.6 | 2.13 |
| 18 | 1.84 | 20.9 | 55.1 | 428.2 | 20.5 | 1.31 |
| 19 | 1.86 | 16.8 | 80.5 | 615 | 36.5 | 1.56 |
| 20 | 0.98 | 12.6 | ND | 1283.8 | 101.9 | 2.01 |
| 21 | 2.33 | 20.2 | 52.6 | 1355.7 | 67.2 | 1.83 |
| 22 | 1.12 | 10.0 | ND | 3044.9 | 304.3 | 2.48 |
| 23 | 2.49 | 6.3 | ND | 8424.4 | 1329.5 | 3.12 |
| 24 | 1.25 | 3.75 | 24.7 | 2595.6 | 691.7 | 2.84 |
| 25 | 2.10 | 29.0 | ND | 2190 | 75.4 | 1.88 |
| 26 | 3.78 | 32.0 | ND | 6043.4 | 188.9 | 2.28 |
| 27 | 10.9 | 42.8 | ND | 11411.2 | 266.4 | 2.43 |
| 28 | 0.89 | 5.5 | ND | 1935.2 | 353.3 | 2.55 |
| 29 | 1.82 | 7.7 | ND | 2826.9 | 366.9 | 2.56 |
| 30 | 3.28 | 36.9 | 204.9 | 1832.1 | 49.6 | 1.70 |
| 31 | 2.40 | 5.5 | 43.6 | 4543.8 | 818.0 | 2.91 |
| 32 | 1.88 | 7.7 | ND | 5782.4 | 747.9 | 2.87 |
| 33 | 21.8 | 55.9 | 380.1 | 3717.8 | 66.5 | 1.82 |
| 34 | 20.9 | 31.7 | 102.7 | 10775.7 | 340.3 | 2.53 |
| 35 | 5.21 | 18.8 | 47.2 | 3942.7 | 209.5 | 2.32 |
| 36 | 3.66 | 56.3 | ND | 4085 | 72.6 | 1.86 |
| 37 | 3.21 | 33.7 | ND | 4077.4 | 121.1 | 2.08 |
| 38 | 3.59 | 17.9 | 122.5 | 4025.1 | 224.3 | 2.35 |
| 39 | 3.47 | 57.8 | ND | 9115.1 | 157.6 | 2.20 |
| 40 | 2.65 | 20.5 | 286.43 | 11645.7 | 568.5 | 2.75 |
| 41 | 6.2 | 65.1 | ND | 12256.1 | 188.2 | 2.27 |
| 42 | 2.9 | 31.7 | ND | 4269.3 | 134.9 | 2.13 |
| 43 | 4.45 | 23.6 | ND | 5359.7 | 227.2 | 2.36 |
| 44 | 4.87 | 29.0 | ND | 6393.2 | 220.4 | 2.34 |
| 45 | 3.25 | 13.6 | 102.7 | 3152.7 | 232.1 | 2.37 |
| 46 | 3.54 | 15.2 | 60.0 | 4323.1 | 284.3 | 2.45 |
| 47 | 4.43 | 20.3 | 93.4 | 3539.4 | 174.6 | 2.24 |
| 48 | 13.5 | 47.9 | ND | >30000.0 | 626.6 | 2.80 |
| 49 | 3.32 | 52.5 | ND | 8750.5 | 166.8 | 2.22 |
| 50 | 0.98 | 5.01 | ND | 743.5 | 148.5 | 2.17 |
| 51 | 27.9 | 85.3 | ND | >30000.0 | 351.6 | 2.55 |
| 52 | 7.6 | 27.7 | 75.3 | 2777.5 | 100.3 | 2.00 |
| 53 | 6.2 | 95.3 | ND | 11414.2 | 119.7 | 2.08 |
| 54 | 9.1 | 35.8 | ND | 5078.4 | 141.9 | 2.15 |
| 55 | 9.0 | 47.8 | ND | 2686.6 | 56.2 | 1.75 |
| 56 | 6.9 | 39.4 | ND | 11461.7 | 290.8 | 2.46 |
| 57 | 1.53 | 7.3 | ND | 4516.9 | 615.2 | 2.79 |
| 58 | 2.59 | 25.0 | ND | 1400 | 56.0 | 1.75 |
| 59 | 2.27 | 8.9 | 21.7 | 1272.7 | 142.9 | 2.16 |
| 60 | 2.86 | 23.4 | ND | 3685.5 | 157.4 | 2.20 |
| 61 | 4.98 | 20.6 | 49.1 | 2524.6 | 122.4 | 2.09 |
| 62 | 5.9 | 24.6 | ND | 4915.4 | 200.3 | 2.30 |
| 63 | 8.1 | 45.9 | ND | 4432 | 96.5 | 1.98 |
| 64 | 6.5 | 56.2 | ND | 7867.6 | 139.9 | 2.15 |
| 65 | 1.69 | 23.1 | ND | 1244.3 | 53.8 | 1.73 |
| 66 | 2.94 | 14.7 | ND | 2505.4 | 170.6 | 2.23 |
| 67 | 7.4 | 21.3 | ND | 1799.9 | 84.5 | 1.93 |
| 68 | 2.95 | 10.6 | ND | 1984.3 | 187.5 | 2.27 |
| 69 | 7.8 | 62.0 | ND | 3702.1 | 59.7 | 1.78 |
| 70 | 5.8 | 49.9 | ND | 3551 | 71.1 | 1.85 |
| 71 | 7.3 | 80.0 | ND | 3918.2 | 49.0 | 1.69 |

### Functional Expression of GABA_{A} Receptors:

Xenopus Oocytes Preparation Xenopus laevis oocytes at maturation stages V-VI were used for the expression of cloned mRNA encoding GABA_{A} receptor subunits. Oocytes ready for RNA micro-injection were bought from Ecocyte, Castrop-Rauxel, Germany and stored in modified Barth's medium (composition in mM: NaCl 88, KCl 1, NaHCO₃ 2.4, HEPES 10, MgSO₄ 0.82, CaNO₃ 0.33, CaCl₂ 0.33, pH=7.5) at 20° C. until the experiment.

### Xenopus Oocytes Microinjection

Oocytes were plated in 96-well plates for microinjection using the Roboinject automated instrument (MultiChannelSystems, Reutlingen, Germany). Approximately 50 nL of an aqueous solution containing the RNA transcripts for the subunits of the desired GABA_{A} receptor subtype was injected into each oocyte. RNA concentrations ranged between 20 and 200 pg/μL/subunit and were adjusted in pilot experiments to obtain GABA responses of a suitable size and a maximal effect of Flunitrazepam, Triazolam and Midazolam, reference benzodiazepine positive allosteric modulators (PAM) at the GABA_{A} receptor benzodiazepine (BZD) binding site. Oocytes were kept in modified Barth's medium (composition in mM: NaCl 88, KCl 1, NaHCO₃ 4, HEPES 10, MgSO₄ 0.82, CaNO₃ 0.33, CaCl₂ 0.33, pH=7.5) at 20° C. until the experiment.

### Electrophysiology

Electrophysiological experiments were performed using the Roboocyte instrument (MultiChannelSystems, Reutlingen, Germany) on days 3 to 5 after the micro-injection of mRNA. During the experiment the oocytes were constantly superfused by a solution containing (in mM) NaCl 90, KCl 1, HEPES 5, MgCl₂ 1, CaCl₂ 1 (pH 7.4). Oocytes were impaled by two glass microelectrodes (resistance: 0.5-0.8 MQ) which were filled with a solution containing KCl 1M+K-acetate 1.5 M and voltage-clamped to −80 mV. The recordings were performed at room temperature using the Roboocyte two-electrode voltage clamp system (Multichannelsystem). After an initial equilibration period of 1.5 min GABA was added for 1.5 min at a concentration evoking approximately 20% of a maximal current response (EC₂₀). After another rest interval of 2.5 min GABA was again added evoking a response of similar amplitude and shape. 0.5 min after the onset of this second GABA application the test compound, at a concentration corresponding to approximatively 30-fold its Kᵢ α2β2γ1, was added while GABA was still present. Current traces were recorded at a digitization rate of 10 Hz during and shortly before and after the GABA application.

Each compound and concentration was tested on at least 3 oocytes. Different oocytes were used for different compound concentrations. The reference PAMs, Flunitrazepam, Triazolam and Midazolam, potentiated the GABA-induced current in α2β2γ1 GABA_{A} receptor subtype expressing oocytes by approximatively 60%.

### Data Analysis

For the analysis, the digitized current traces of the first and second GABA response were superimposed and, if necessary, rescaled to equal maximal amplitudes. The ratio between the two responses during the time interval of test compound application was calculated point by point. The extremum of the resulting “ratio trace” was taken as the efficacy (“Fold increase”) of the compound expressed as “% modulation of GABA EC₂₀” (100*(Fold increase−1)).

The results are shown in Table 2.

TABLE 2

| | Ki | Fold increase | |
|---|---|---|---|
| | h-GABA_{A} | h-GABA-A | |
| | α2β2γ1 | α2β2γ1 | Efficacy |
| Example | (nM) | oocyte @ 30-fold Ki | (GABA) % |
| 1 | 7.5 | 1.64 | 64 |
| 2 | 7.4 | 1.66 | 66 |
| 3 | 26.7 | 1.91 | 91 |
| 4 | 47.8 | 2.19 | 119 |
| 5 | 63.5 | ND | — |
| 6 | 37.3 | ND | — |
| 7 | 37.5 | 2.69 | 169 |
| 8 | 51.3 | 3.03 | 203 |
| 9 | 14.3 | 1.96 | 96 |
| 10 | 18.2 | ND | — |
| 11 | 17.5 | 1.88 | 88 |
| 12 | 33.7 | 2.85 | 185 |
| 13 | 15.1 | 1.80 | 80 |
| 14 | 38.4 | 2.81 | 181 |
| 15 | 42.8 | 2.44 | 144 |
| 16 | 89.8 | ND | — |
| 17 | 4.3 | 2.35 | 135 |
| 18 | 20.9 | 2.13 | 113 |
| 19 | 16.9 | 2.22 | 122 |
| 20 | 12.6 | 1.76 | 76 |
| 21 | 20.2 | 2.40 | 140 |
| 22 | 10.0 | 1.88 | 88 |
| 23 | 6.3 | 1.63 | 63 |
| 24 | 3.8 | 1.59 | 59 |
| 25 | 29.0 | 1.99 | 99 |
| 26 | 32.0 | 2.09 | 109 |
| 27 | 42.8 | 1.77 | 77 |
| 28 | 5.5 | 1.40 | 40 |
| 29 | 7.7 | 1.79 | 79 |
| 30 | 36.9 | 2.50 | 150 |
| 31 | 5.6 | 1.69 | 69 |
| 32 | 7.7 | ND | — |
| 33 | 55.9 | 1.76 | 76 |
| 34 | 31.7 | 2.34 | 134 |
| 35 | 18.8 | 1.61 | 61 |
| 36 | 56.3 | 1.89 | 89 |
| 37 | 33.7 | 1.86 | 86 |
| 38 | 17.9 | ND | — |
| 39 | 57.8 | 2.50 | 150 |
| 40 | 20.5 | 2.41 | 141 |
| 41 | 65.1 | 2.67 | 167 |
| 42 | 31.7 | 2.37 | 137 |
| 43 | 23.6 | 1.94 | 94 |
| 44 | 29.0 | 2.16 | 116 |
| 45 | 13.6 | 2.01 | 101 |
| 46 | 15.2 | 1.84 | 84 |
| 47 | 20.3 | 1.89 | 89 |
| 48 | 47.9 | 2.44 | 144 |
| 49 | 52.5 | 2.31 | 131 |
| 50 | 5.0 | 1.55 | 55 |
| 51 | 85.3 | 2.08 | 108 |
| 52 | 27.7 | 2.70 | 170 |
| 53 | 95.3 | 3.37 | 237 |
| 54 | 35.8 | ND | — |
| 55 | 47.8 | ND | — |
| 56 | 39.4 | 2.47 | 147 |
| 57 | 7.3 | 1.73 | 73 |
| 58 | 25.0 | ND | — |
| 59 | 8.9 | ND | — |
| 60 | 23.4 | ND | — |
| 61 | 20.6 | 2.65 | 165 |
| 62 | 24.5 | 2.79 | 179 |
| 63 | 45.9 | 2.69 | 169 |
| 64 | 56.2 | 2.64 | 164 |
| 65 | 23.1 | 2.69 | 169 |
| 66 | 14.7 | 2.53 | 153 |
| 67 | 21.3 | 2.64 | 164 |
| 68 | 10.6 | 2.59 | 159 |
| 69 | 62.0 | ND | — |
| 70 | 49.9 | 2.48 | 148 |
| 71 | 80.0 | ND | — |

### Reference Compounds

Benzodiazepines reference compounds (classical marketed benzodiazepines) and their structural analogues listed below were tested for their affinity towards the GABA_{A} receptor α1β2γ1 and α2β2γ1 subtypes as well as in the GABA_{A} receptor α1β3γ2 subtype.

U.S. Pat. No. 4,621,083 discloses reference compound RE-A. Reference example RE-B has been prepared as described herein. The results are shown in Table 3.

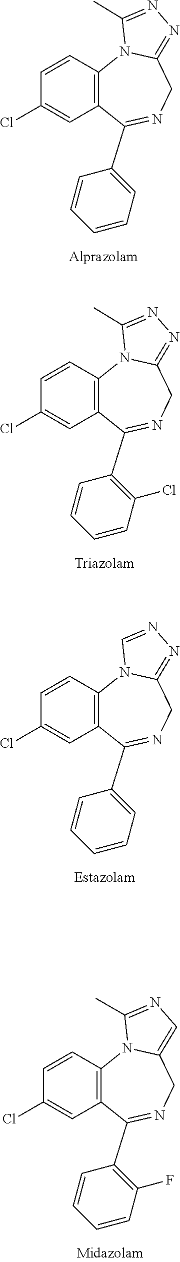 [SMILES: Cc1nnc2n1-c1ccc(Cl)cc1C(c1ccccc1)=NC2 ; Cc1nnc2n1-c1ccc(Cl)cc1C(c1ccccc1Cl)=NC2 ; Clc1ccc2c(c1)C(c1ccccc1)=NCc1nncn1-2 ; Cc1ncc2n1-c1ccc(Cl)cc1C(c1ccccc1F)=NC2]

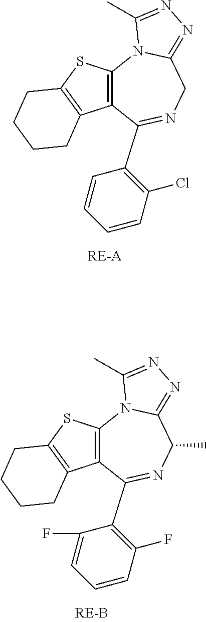 [SMILES: Cc1nnc2n1-c1sc3c(c1C(c1ccccc1Cl)=NC2)CCCC3 ; Cc1nnc2n1-c1sc3c(c1C(c1c(F)cccc1F)=N[C@H]2C)CCCC3]

TABLE 3

| | Ki | Ki | Ki | | |
|---|---|---|---|---|---|
| | h-GABA_{A} | h-GABA_{A} | h-GABA_{A} | γ2/γ1 | |
| | α1β2γ1 | α2β2γ1 | α1β3γ2 | Selectivity | |
| Example | (nM) | (nM) | (nM) | Ratio | LogSel |
| Alprazolam | 5923 | 3945 | 19.6 | 0.0050 | −2.3 |
| Triazolam | 44.2 | 46.2 | 1.5 | 0.032 | −1.5 |
| Estazolam | ND | 3182 | 28.4 | 0.0089 | −2.0 |
| Midazolam | 1153.2 | 737.7 | 5.0 | 0.0068 | −2.2 |
| RE-A | ND | 52.6 | 48.5 | 0.922 | −0.035 |
| RE-B | ND | 57.7 | 21.6 | 2.68 | 0.43 |
| Example 1 | 19.7 | 7.5 | 373.5 | 49.6 | 1.7 |
| Example 21 | 52.6 | 20.2 | 1355.7 | 67.2 | 1.8 |
| Example 52 | 75.3 | 27.7 | 2777.5 | 100.3 | 2.0 |

### Preparation of Pharmaceutical Compositions Comprising Compounds of the Invention

Tablets comprising compounds of formula (I) are manufactured as follows:

mg/tablet

| Ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

Capsules comprising compounds of formula (I) are manufactured as follows:

mg/capsule

| Ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

A compound of formula I lactose and corn starch are firstly mixed in a mixer and then in a comminuting machine. The mixture is returned to the mixer; the talc is added thereto and mixed thoapproximatively. The mixture is filled by machine into suitable capsules, e.g. hard gelatin capsules.

Injection solutions comprising compounds of formula (I) are manufactured as follows:

| Ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

1. A compound of formula (I) 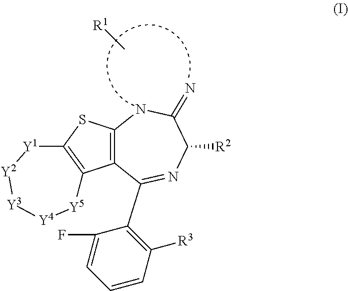
or a pharmaceutically acceptable salt thereof, wherein: 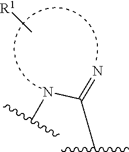
is selected from: 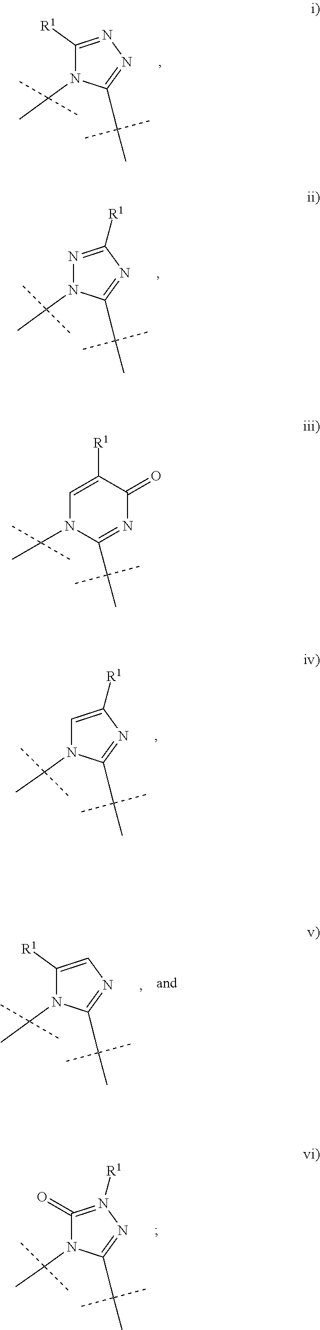
R¹ is selected from hydrogen, C₁-C₆-alkyl, halo-C₁-C₆-alkyl, R^{1a}R^{1b}NC(O)—, C₃-C₁₀-cycloalkyl, 3-14-membered heteroaryl;
R^{1a} is selected from C₁-C₆-alkyl, hydroxy-C₁-C₆-alkyl, halo-C₁-C₆-alkyl, halo-hydroxy-C₁-C₆-alkyl, C₃-C₁₀-cycloalkyl, hydroxy-C₃-C₁₀-cycloalkyl-C₁-C₆-alkyl-; and R^{1b} is hydrogen; or
R^{1a} and R^{1b}, taken together with the nitrogen atom to which they are attached, form a 3-14-membered heterocycle which is optionally substituted with one substituent selected from halogen, hydroxy, and C₁-C₆-alkoxy;
R² is selected from hydrogen and C₁-C₆-alkyl;
R³ is selected from chloro and fluoro;
Y¹ to Y⁵ are each independently selected from O, S, and CR⁴R⁵, provided that at most two of Y¹ to Y⁵ are O or S and that an Y being O or S is not bound to another Y also being O or S;
R⁴ and R⁵ are each independently selected from hydrogen, deuterium, halogen, and hydroxy.

2. The compound of formula (I) according to claim 1, or a pharmaceutically acceptable salt thereof, wherein 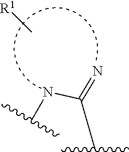
is selected from: 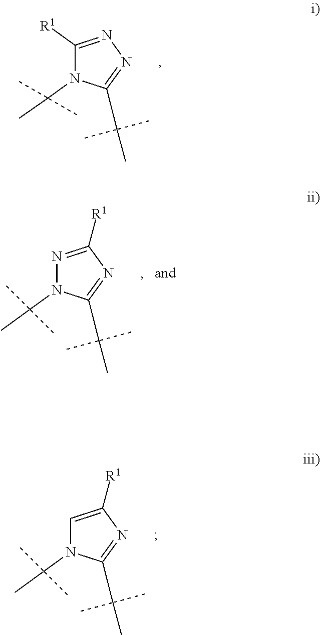
R¹ is selected from C₁-C₆-alkyl and R^{1a}R^{1b}NC(O)—;
R^{1a} is selected from hydroxy-C₁-C₆-alkyl and halo-hydroxy-C₁-C₆-alkyl; and
R^{1b} is hydrogen.

3. The compound of formula (I) according to claim 2, or a pharmaceutically acceptable salt thereof, wherein 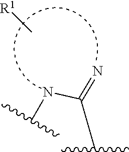
is selected from: 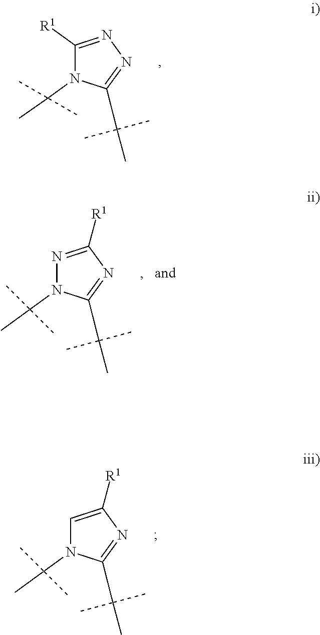
R¹ is selected from methyl and R^{1a}R^{1b}NC(O)—;
R^{1a} is selected from 2-hydroxy-2-methyl-propyl and 3,3,3-trifluoro-2-hydroxy-2-methyl-propyl; and
R^{1b} is hydrogen.

4. The compound of formula (I) according to claim 1, or a pharmaceutically acceptable salt thereof, wherein R² is C₁-C₆-alkyl.

5. The compound of formula (I) according to claim 4, or a pharmaceutically acceptable salt thereof, wherein R² is methyl.

6. The compound of formula (I) according to claim 1, or a pharmaceutically acceptable salt thereof, wherein R³ is fluoro.

7. The compound of formula (I) according to claim 1, or a pharmaceutically acceptable salt thereof, wherein:
Y¹, Y³, Y⁴, and Y⁵ are each independently selected from O and CR⁴R⁵, provided that at most two of Y, Y³, Y⁴, and Y⁵ are O and that an Y being O is not bound to another Y also being O;
Y² is CR⁴R⁵;
R⁴ is selected from hydrogen, deuterium, halogen, and hydroxy; and
R⁵ is selected from hydrogen, deuterium, and halogen.

8. The compound of formula (I) according to claim 7, or a pharmaceutically acceptable salt thereof, wherein:
Y¹ and Y⁴ are each independently selected from O and CR⁴R⁵;
Y², Y³, and Y⁵ are each CR⁴R⁵;
R⁴ is selected from hydrogen and halogen; and
R⁵ is hydrogen.

9. The compound of formula (I) according to claim 8, or a pharmaceutically acceptable salt thereof, wherein:
Y¹ and Y⁴ are each independently selected from O and CR⁴R⁵;
Y², Y³, and Y⁵ are each CR⁴R⁵;
R⁴ is selected from hydrogen and fluoro; and
R⁵ is hydrogen.

10. The compound of formula (I) according to claim 1, or a pharmaceutically acceptable salt thereof, wherein: 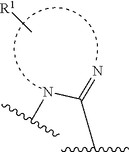
is selected from: 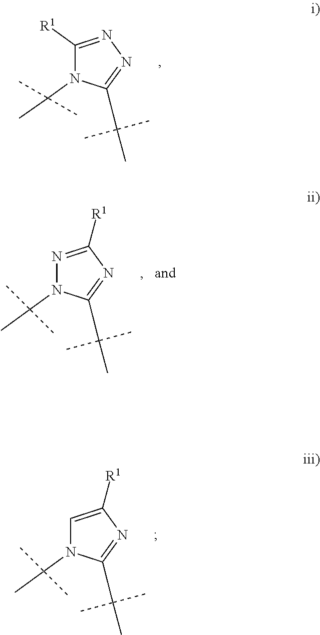
R¹ is selected from C₁-C₆-alkyl and R^{1a}R^{1b}NC(O)—;
R^{1a} is selected from hydroxy-C₁-C₆-alkyl and halo-hydroxy-C₁-C₆-alkyl;
R^{1b} is hydrogen;
R² is C₁-C₆-alkyl;
R³ is fluoro;
Y¹ and Y⁴ are each independently selected from O and CR⁴R⁵;
Y², Y³, and Y⁵ are each CR⁴R⁵;
R⁴ is selected from hydrogen and halogen; and
R⁵ is hydrogen.

11. The compound of formula (I) according to claim 10, or a pharmaceutically acceptable salt thereof, wherein: 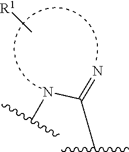
is selected from: 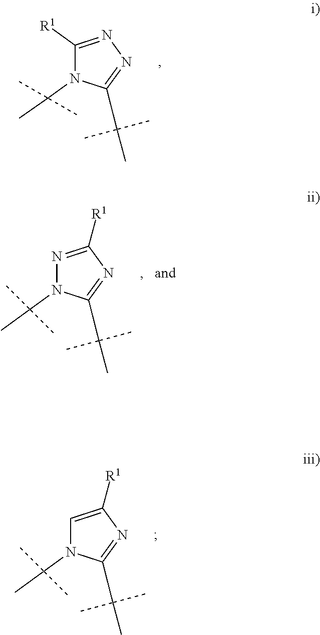
R¹ is selected from methyl and R^{1a}R^{1b}NC(O)—;
R^{1a} is selected from 2-hydroxy-2-methyl-propyl and 3,3,3-trifluoro-2-hydroxy-2-methyl-propyl;
R^{1b} is hydrogen;
R² is methyl;
R³ is fluoro;
Y¹ and Y⁴ are each independently selected from O and CR⁴R⁵;
Y², Y³, and Y⁵ are each CR⁴R⁵;
R⁴ is selected from hydrogen and fluoro; and
R⁵ is hydrogen.

12. A compound, or a pharmaceutically acceptable salt thereof, wherein said compound is selected from:
9-(2,6-difluorophenyl)-3-methyl-18-thia-2,4,5,8-tetrazatetracyclo[8.8.0.02,6.011,17]octadeca-1(10),3,5,8,11(17)-pentaene;
(7S)-9-(2,6-difluorophenyl)-3,7-dimethyl-18-thia-2,4,5,8-tetrazatetracyclo[8.8.0.02,6.011,17]octadeca-1(10),3,5,8,11(17)-pentaene;
(7S)-3-cyclopropyl-9-(2,6-difluorophenyl)-7-methyl-18-thia-2,4,5,8-tetrazatetracyclo[8.8.0.02,6.011,17]octadeca-1(10),3,5,8,11(17)-pentaene;
(7S)-9-(2,6-difluorophenyl)-7-methyl-3-pyridazin-3-yl-18-thia-2,4,5,8-tetrazatetracyclo[8.8.0.02,6.011,17]octadeca-1(10),3,5,8,11(17)-pentaene;
9-(2,6-difluorophenyl)-3-methyl-14-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-7-methyl-18-thia-2,4,5,8-tetrazatetracyclo[8.8.0.02,6.011,17]octadeca-1(10),5,8,11(17)-tetraen-3-one;
(8S)-10-(2,6-difluorophenyl)-8-methyl-19-thia-2,6,9-triazatetracyclo[9.8.0.02,7.012,18]nonadeca-1(11),3,6,9,12(18)-pentaen-5-one;
(7S)-9-(2,6-difluorophenyl)-15,15-difluoro-3,7-dimethyl-18-thia-2,4,5,8-tetrazatetracyclo[8.8.0.02,6.011,17]octadeca-1(10),3,5,8,11(17)-pentaene;
(7S,11RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-16-ol;
(7S,16RS)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,16R)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,16S)-9-(2,6-difluorophenyl)-16-fluoro-3,7-dimethyl-18-thia-2,4,5,8-tetrazatetracyclo[8.8.0.02,6.011,17]octadeca-1(10),3,5,8,11(17)-pentaene;
(7S,15RS)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-15-ol;
(7S,14S)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene;
(7S,14R)-9-(2,6-difluorophenyl)-14-fluoro-3,7-dimethyl-18-thia-2,4,5,8-tetrazatetracyclo[8.8.0.02,6.011,17]octadeca-1(10),3,5,8,11(17)-pentaene;
(7S,15R)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
(7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
9-(2,6-difluorophenyl)-3-methyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-3,7-dimethyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-4,7-dimethyl-18-thia-2,3,5,8-tetrazatetracyclo[8.8.0.02,6.011,17]octadeca-1(10),3,5,8,11(17)-pentaene;
(7S)-9-(2,6-difluorophenyl)-3,7-dimethyl-13,16-dioxa-18-thia-2,4,5,8-tetrazatetracyclo[8.8.0.02,6.011,17]octadeca-1(10),3,5,8,11(17)-pentaene;
azetidin-1-yl-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
azetidin-1-yl-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(3-fluoroazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-N-(2-fluoroethyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(8S)-10-(2,6-difluorophenyl)-4,8-dimethyl-19-thia-2,6,9-triazatetracyclo[9.8.0.02,7.012,18]nonadeca-1(11),3,6,9,12(18)-pentaen-5-one;
(7S,14R)-9-(2,6-difluorophenyl)-3,7-dimethyl-18-thia-2,4,5,8-tetrazatetracyclo[8.8.0.02,6.011,17]octadeca-1(10),3,5,8,11(17)-pentaen-14-ol;
(7S)-9-(2,6-difluorophenyl)-4,7-dimethyl-13,16-dioxa-18-thia-2,3,5,8-tetrazatetracyclo[8.8.0.02,6.011,17]octadeca-1(10),3,5,8,11(17)-pentaene;
(3-tert-butoxyazetidin-1-yl)-[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S,15S)-9-(2,6-difluorophenyl)-3,7,15-trimethyl-13,16-dioxa-18-thia-2,4,5,8-tetrazatetracyclo[8.8.0.02,6.011,17]octadeca-1(10),3,5,8,11(17)-pentaene;
[(7S)-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]-(3-ethoxyazetidin-1-yl)methanone;
(3-ethoxyazetidin-1-yl)-[(7S)-14,14,15,15-tetradeuterio-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-3,7-dimethyl-12-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-14,15-difluoro-3,7-dimethyl-18-thia-2,4,5,8-tetrazatetracyclo[8.8.0.02,6.011,17]octadeca-1(10),3,5,8,11(17)-pentaene;
(7S)-9-(2-chloro-6-fluoro-phenyl)-3,7-dimethyl-13,16-dioxa-18-thia-2,4,5,8-tetrazatetracyclo[8.8.0.02,6.011,17]octadeca-1(10),3,5,8,11(17)-pentaene;
(7S)—N-cyclopropyl-9-(2,6-difluorophenyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-14,14,15,15-tetradeuterio-9-(2,6-difluorophenyl)-N-ethyl-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S,15S)-9-(2,6-difluorophenyl)-4,7,15-trimethyl-13,16-dioxa-18-thia-2,3,5,8-tetrazatetracyclo[8.8.0.02,6.011,17]octadeca-1(10),3,5,8,11(17)-pentaene;
[(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]-(3-fluoroazetidin-1-yl)methanone;
(3-tert-butoxyazetidin-1-yl)-[(7S,15S)-9-(2,6-difluorophenyl)-7,15-dimethyl-13,16-dioxa-18-thia-2,3,5,8-tetrazatetracyclo[8.8.0.02,6.011,17]octadeca-1(10),3,5,8,11(17)-pentaen-4-yl]methanone;
(7S)-9-(2,6-difluorophenyl)-3-ethyl-7-methyl-13,16-dioxa-18-thia-2,4,5,8-tetrazatetracyclo[8.8.0.02,6.011,17]octadeca-1(10),3,5,8,11(17)-pentaene;
(7S)-3-cyclopropyl-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene;
(7S)-9-(2,6-difluorophenyl)-3-isopropyl-7-methyl-13,16-dioxa-18-thia-2,4,5,8-tetrazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-3-(difluoromethyl)-9-(2,6-difluorophenyl)-7-methyl-13,16-dioxa-18-thia-2,4,5,8-tetrazatetracyclo[8.8.0.02,6.011,17]octadeca-1(10),3,5,8,11(17)-pentaene;
(7S,15S)-9-(2,6-difluorophenyl)-4-ethyl-7,15-dimethyl-13,16-dioxa-18-thia-2,3,5,8-tetrazatetracyclo[8.8.0.02,6.011,17]octadeca-1(10),3,5,8,11(17)-pentaene;
(7S)-9-(2,6-difluorophenyl)-3,7-dimethyl-13,16-dioxa-18-thia-2,5,8-triazatetracyclo[8.8.0.02,6.011,17]octadeca-1(10),3,5,8,11(17)-pentaene;
(7S,14R)-9-(2,6-difluorophenyl)-3,7-dimethyl-18-thia-2,5,8-triazatetracyclo[8.8.0.02,6.011,17]octadeca-1(10),3,5,8,11(17)-pentaen-14-ol;
(7S)-9-(2,6-difluorophenyl)-3,7-dimethyl-13-oxa-18-thia-2,4,5,8-tetrazatetracyclo[8.8.0.02,6.011,17]octadeca-1(10),3,5,8,11(17)-pentaene;
(7S,15S)-9-(2,6-difluorophenyl)-N-(2-hydroxyethyl)-7,15-dimethyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-16-fluoro-3,7-dimethyl-13-oxa-18-thia-2,4,5,8-tetrazatetracyclo[8.8.0.02,6.011,17]octadeca-1(10),3,5,8,11(17)-pentaene;
(7S,14R)-9-(2,6-difluorophenyl)-14-fluoro-3,7-dimethyl-16-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-4,7-dimethyl-13-oxa-18-thia-2,3,5,8-tetrazatetracyclo[8.8.0.02,6.011,17]octadeca-1(10),3,5,8,11(17)-pentaene;
(7S)-9-(2,6-difluorophenyl)-4,7-dimethyl-13-oxa-18-thia-2,5,8-triazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2-chloro-6-fluoro-phenyl)-3,7-dimethyl-13-oxa-18-thia-2,4,5,8-tetrazatetracyclo[8.8.0.02,6.011,17]octadeca-1(10),3,5,8,11(17)-pentaene;
(7S)-9-(2,6-difluorophenyl)-N-[(1-hydroxycyclopropyl)methyl]-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-(trans-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-(cis-3-hydroxycyclobutyl)-7-methyl-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13,16-dioxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-(2-hydroxyethyl)-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-(2-hydroxy-2-methyl-propyl)-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-N-[(1-hydroxycyclopropyl)methyl]-7-methyl-13-oxa-18-thia-2,5,8-triazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13-oxa-18-thia-2,3,5,8-tetrazatetracyclo[8.8.0.02,6.011,17]octadeca-1(10),3,5,8,11(17)-pentaene-4-carboxamide; and
(7S)-9-(2,6-difluorophenyl)-N-(2-hydroxy-2-methyl-propyl)-7-methyl-13-oxa-18-thia-2,3,5,8-tetrazatetracyclo[8.8.0.02,6.011,17]octadeca-1(10),3,5,8,11(17)-pentaene-4-carboxamide.

13. The compound of formula (I) according to claim 12, or a pharmaceutically acceptable salt thereof, wherein said compound of formula (I) is selected from:
(7S,15S)-9-(2,6-difluorophenyl)-15-fluoro-3,7-dimethyl-18-thia-2,4,5,8-tetrazatetracyclo[8.8.0.02,6.011,17]octadeca-1(10),3,5,8,11(17)-pentaene;
(7S)-9-(2,6-difluorophenyl)-N-(2-hydroxy-2-methyl-propyl)-7-methyl-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2R)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide;
(7S)-9-(2,6-difluorophenyl)-7-methyl-N-[(2S)-3,3,3-trifluoro-2-hydroxy-2-methyl-propyl]-13,16-dioxa-18-thia-2,3,5,8-tetrazatetracyclo[8.8.0.02,6.011,17]octadeca-1(10),3,5,8,11(17)-pentaene-4-carboxamide; and
(7S)-9-(2,6-difluorophenyl)-N-(2-hydroxy-2-methyl-propyl)-7-methyl-13,16-dioxa-18-thia-2,5,8-triazatetracyclo[8.8.0.02,6.011,17]octadeca-1(10),3,5,8,11(17)-pentaene-4-carboxamide.

14. A pharmaceutical composition comprising a compound of formula (I) according to claim 1, or a pharmaceutically acceptable salt thereof, and a therapeutically inert carrier.

15. A method for treating or preventing acute neurological disorders, chronic neurological disorders and/or cognitive disorders in a subject, said method comprising administering an effective amount of a compound of formula (I) according to claim 1, or a pharmaceutically acceptable salt thereof, to the subject.

16. The method according to claim 15, wherein said acute neurological disorders, chronic neurological disorders and/or cognitive disorders are selected from autism spectrum disorders (ASD), Angelman syndrome, age-related cognitive decline, Rett syndrome, Prader-Willi syndrome, amyotrophic lateral sclerosis (ALS), fragile-X disorder, negative and/or cognitive symptoms associated with schizophrenia, tardive dyskinesia, anxiety, social anxiety disorder (social phobia), panic disorder, agoraphobia, generalized anxiety disorder, disruptive, impulse-control and conduct disorders, Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurodegenerative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar disorders, epilepsy and pain.
